SEQUENCE LISTING (39 sequences)

SEQ ID NO: 1 - RNA, 20 nt, synthetic construct
  misc_feature 1..20: note = Description of Artificial Sequence: Synthetic oligonucleotide
  source 1..20: mol_type = other RNA
ccgacttctgaacgtgcggt

SEQ ID NO: 2 - RNA, 20 nt, synthetic construct
  misc_feature 1..20: note = Description of Artificial Sequence: Synthetic oligonucleotide
  source 1..20: mol_type = other RNA
tcacccgacttctgaacgtg

SEQ ID NO: 3 - RNA, 20 nt, synthetic construct
  misc_feature 1..20: note = Description of Artificial Sequence: Synthetic oligonucleotide
  source 1..20: mol_type = other RNA
atgatagtcactgacaacaa

SEQ ID NO: 4 - protein, 4 aa, synthetic construct
  REGION 1..4: note = Description of Artificial Sequence: Synthetic peptide
RRRR

SEQ ID NO: 5 - protein, 4 aa, synthetic construct
  REGION 1..4: note = Description of Artificial Sequence: Synthetic peptide
RRKR

SEQ ID NO: 6 - protein, 4 aa, synthetic construct
  REGION 1..4: note = Description of Artificial Sequence: Synthetic peptide
RKRR

SEQ ID NO: 7 - protein, 4 aa, synthetic construct
  REGION 1..4: note = Description of Artificial Sequence: Synthetic peptide
RKKR

SEQ ID NO: 8 - protein, 565 aa, synthetic construct
  REGION 1..565: note = Description of Artificial Sequence: Synthetic polypeptide
MGRGLLRGLWPLHIVLWTRIASTIPPHVQKSVNNDMIVTDNNGAVKFPQLCKFCDVRFTCDNQKSCMSNCSITSICEKPQEVCVAVWRKNDENITLETVCHDPKLPYHDFILEDAASPCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPQARLTAQCVAERFSELEHLDRLSGRSCSEEKIPEDGSLNTTK

SEQ ID NO: 9 - protein, 21 aa, synthetic construct
  REGION 1..21: note = Description of Artificial Sequence: Synthetic peptide
MALPVTALLLPLALLLHAARP

SEQ ID NO: 10 - protein, 161 aa, synthetic construct
  REGION 1..161: note = Description of Artificial Sequence: Synthetic polypeptide
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 11 - protein, 21 aa, synthetic construct
  REGION 1..21: note = Description of Artificial Sequence: Synthetic peptide
IYIWAPLAGTCGVLLLSLVIT

SEQ ID NO: 12 - protein, 32 aa, synthetic construct
  REGION 1..32: note = Description of Artificial Sequence: Synthetic polypeptide
LYCNHRNRRRVCKCPRPVVKSGDKPSLSARYV

SEQ ID NO: 13 - protein, 21 aa, synthetic construct
  REGION 1..21: note = Description of Artificial Sequence: Synthetic peptide
MRPRLWLLLAAQLTVLHGNSV

SEQ ID NO: 14 - protein, 149 aa, synthetic construct
  REGION 1..149: note = Description of Artificial Sequence: Synthetic polypeptide
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 15 - protein, 21 aa, synthetic construct
  REGION 1..21: note = Description of Artificial Sequence: Synthetic peptide
ITLGLLVAGVLVLLVSLGVAI

SEQ ID NO: 16 - protein, 19 aa, synthetic construct
  REGION 1..19: note = Description of Artificial Sequence: Synthetic peptide
HLCCRRRRARLRFMKQFYK

SEQ ID NO: 17 - protein, 40 aa, synthetic construct
  REGION 1..40: note = Description of Artificial Sequence: Synthetic polypeptide
CVRCRHRRRQAERMSQIKRLLSEKKTCQCPHRFQKTCSPI

SEQ ID NO: 18 - protein, 19 aa, synthetic construct
  REGION 1..19: note = Description of Artificial Sequence: Synthetic peptide
ATNFSLLKQAGDVEENPGP

SEQ ID NO: 19 - protein, 22 aa, synthetic construct
  REGION 1..22: note = Description of Artificial Sequence: Synthetic peptide
GSGATNFSLLKQAGDVEENPGP

SEQ ID NO: 20 - DNA, 225 bp, synthetic construct
  misc_feature 1..225: note = Description of Artificial Sequence: Synthetic polynucleotide
  source 1..225: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 21 - DNA, 134 bp, synthetic construct
  misc_feature 1..134: note = Description of Artificial Sequence: Synthetic polynucleotide
  source 1..134: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 22 - DNA, 44 bp, synthetic construct
  misc_feature 1..44: note = Description of Artificial Sequence: Synthetic oligonucleotide
  source 1..44: mol_type = other DNA
tttatttgtgaaatttgtgatgctattgctttatttgtaaccat

SEQ ID NO: 23 - skipped

SEQ ID NO: 24 - DNA, 244 bp, synthetic construct
  misc_feature 1..244: note = Description of Artificial Sequence: Synthetic polynucleotide
  source 1..244: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctt

SEQ ID NO: 25 - DNA, 1010 bp, synthetic construct
  misc_feature 1..1010: note = Description of Artificial Sequence: Synthetic polynucleotide
  source 1..1010: mol_type = other DNA
cggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatttaaataaacttataaattgtgagagaaattaatgaatgtctaagttaatgcagaaacggaggctcctcatttatttttgaacttaaagacttaatattgtgaaggtatactttctttaataataagcctgcgcccaatatgttcaccccaaaaaagctgtttgttaacttgtcaacctcatttaaaatatataagaaacagcccaaagacaataacaaaagaataataaaaaagaatgaaatatgtaattctttcagagtaaaaatcacacccatgacctggccactgagggcttgatcaattcactttgaatttggcattaaataccattaaggtatattaactgattttaaaataagatatattc

SEQ ID NO: 26 - DNA, 249 bp, synthetic construct
  misc_feature 1..249: note = Description of Artificial Sequence: Synthetic polynucleotide
  source 1..249: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtg

SEQ ID NO: 27 - DNA, 143 bp, synthetic construct
  misc_feature 1..143: note = Description of Artificial Sequence: Synthetic polynucleotide
  source 1..143: mol_type = other DNA
cctggccttgggggagggggaggccagaatgagagctcctggccttgggggagggggaggccagaatgactcgacctggccttgggggagggggaggccagaatgagagctcctggccttgggggagggggaggccagaatga

SEQ ID NO: 28 - DNA, 1698 bp, synthetic construct
  misc_feature 1..1698: note = Description of Artificial Sequence: Synthetic polynucleotide
  source 1..1698: mol_type = other DNA
atgggccgcggcctgctgcgcggcctgtggccgctgcatattgtgctgtggacccgcattgcgagcaccattccgccgcatgtgcagaaaagcgtgaacaacgatatgattgtgaccgataacaacggcgcggtgaaatttccgcagctgtgcaaattttgcgatgtgcgctttacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgtgcattatgaaagaaaaaaaaaaaccgggcgaaaccttttttatgtgcagctgcagcagcgatgaatgcaacgataacattatttttagcgaagaatataacaccagcaacccggatctgctgctggtgatttttcaggtgaccggcattagcctgctgccgccgctgggcgtggcgattagcgtgattattattttttattgctatcgcgtgaaccgccagcagaaactgagcagcacctgggaaaccggcaaaacccgcaaactgatggaatttagcgaacattgcgcgattattctggaagatgatcgcagcgatattagcagcacctgcgcgaacaacattaaccataacaccgaactgctgccgattgaactggataccctggtgggcaaaggccgctttgcggaagtgtataaagcgaaactgaaacagaacaccagcgaacagtttgaaaccgtggcggtgaaaatttttccgtatgaagaatatgcgagctggaaaaccgaaaaagatatttttagcgatattaacctgaaacatgaaaacattctgcagtttctgaccgcggaagaacgcaaaaccgaactgggcaaacagtattggctgattaccgcgtttcatgcgaaaggcaacctgcaggaatatctgacccgccatgtgattagctgggaagatctgcgcaaactgggcagcagcctggcgcgcggcattgcgcatctgcatagcgatcataccccgtgcggccgcccgaaaatgccgattgtgcatcgcgatctgaaaagcagcaacattctggtgaaaaacgatctgacctgctgcctgtgcgattttggcctgagcctgcgcctggatccgaccctgagcgtggatgatctggcgaacagcggccaggtgggcaccgcgcgctatatggcgccggaagtgctggaaagccgcatgaacctggaaaacgtggaaagctttaaacagaccgatgtgtatagcatggcgctggtgctgtgggaaatgaccagccgctgcaacgcggtgggcgaagtgaaagattatgaaccgccgtttggcagcaaagtgcgcgaacatccgtgcgtggaaagcatgaaagataacgtgctgcgcgatcgcggccgcccggaaattccgagcttttggctgaaccatcagggcattcagatggtgtgcgaaaccctgaccgaatgctgggatcatgatccgcaggcgcgcctgaccgcgcagtgcgtggcggaacgctttagcgaactggaacatctggatcgcctgagcggccgcagctgcagcgaagaaaaaattccggaagatggcagcctgaacaccaccaaataa

SEQ ID NO: 29 - DNA, 1000 bp, synthetic construct
  misc_feature 1..1000: note = Description of Artificial Sequence: Synthetic polynucleotide
  source 1..1000: mol_type = other DNA
acattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattc

SEQ ID NO: 30 - DNA, 1000 bp, synthetic construct
  misc_feature 1..1000: note = Description of Artificial Sequence: Synthetic polynucleotide
  source 1..1000: mol_type = other DNA
accgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacag

SEQ ID NO: 31-32 - skipped (2 entries)

SEQ ID NO: 33 - DNA, 57 bp, synthetic construct
  misc_feature 1..57: note = Description of Artificial Sequence: Synthetic oligonucleotide
  source 1..57: mol_type = other DNA
gccaccaacttcagcctgctgaagcaggccggcgacgtggaggagaaccccggccct

SEQ ID NO: 34 - DNA, 57 bp, synthetic construct
  misc_feature 1..57: note = Description of Artificial Sequence: Synthetic oligonucleotide
  source 1..57: mol_type = other DNA
gccactaacttctccctgttgaaacaggctggcgatgttgaagaaaaccccggtcct

SEQ ID NO: 35 - protein, 26 aa, synthetic construct
  REGION 1..26: note = Description of Artificial Sequence: Synthetic peptide
MATGSRTSLLLAFGLLCLPWLQEGSA

SEQ ID NO: 36 - DNA, 78 bp, synthetic construct
  misc_feature 1..78: note = Description of Artificial Sequence: Synthetic oligonucleotide
  source 1..78: mol_type = other DNA
atggccacaggcagcagaacatctctgctgctggccttcggactgctgtgtctgccttggctgcaagagggttccgcc

SEQ ID NO: 37 - DNA, 78 bp, synthetic construct
  misc_feature 1..78: note = Description of Artificial Sequence: Synthetic oligonucleotide
  source 1..78: mol_type = other DNA
atggccaccggctctagaacaagcctgctgctcgcttttggcctgctctgcctcccatggctccaagaaggatctgct

SEQ ID NO: 38 - protein, 196 aa, synthetic construct
  REGION 1..196: note = Description of Artificial Sequence: Synthetic polypeptide
MGRGLLRGLWPLHIVLWTRIASTIPPHVQKSVNNDMIVTDNNGAVKFPQLCKFCDVRFTCDNQKSCMSNCSITSICEKPQEVCVAVWRKNDENITLETVCHDPKLPYHDFILEDAASPCIMKEKKKPGETFFMCSCSSDECNDNIIFSEEYNTSNPDLLLVIFQVTGISLLPPLGVAISVIIIFYCYRVNRQQKLS

SEQ ID NO: 39 - DNA, 669 bp, synthetic construct
  misc_feature 1..669: note = Description of Artificial Sequence: Synthetic polynucleotide
  source 1..669: mol_type = other DNA
atgggacgcgggctgttgcggggtctctggccactgcatatcgtgctttggaccaggatcgcatctacaattccgccgcacgtacaaaagtcagatgtggagatggaggcccaaaaagacgaaattatttgcccttcttgtaacaggacagctcacccacttcgccacatcaacaatgacatgatagtgaccgacaataacggagcagtaaaattccctcagttgtgcaagttctgtgacgtgcgattcagcacttgtgataaccagaagtcctgtatgtctaattgttccatcacgtctatttgcgaaaaaccgcaagaagtctgcgtcgcggtatggcggaagaacgacgaaaatattacgcttgagacagtttgtcacgatccaaaacttccgtaccatgacttcattcttgaggatgcggcaagccccaagtgcattatgaaggagaagaagaaacctggagaaaccttcttcatgtgcagttgttcctccgacgaatgtaatgacaacattattttttcagaggaatataatacctccaatccagacttgttgctggtaatcttccaagtgacgggaatcagtttgcttccgcctcttggggtagccatctcagttataattatcttttactgctatcgcgttaaccggcaacaaaaattgagt

(19) United States
(12) Patent Application Publication
(10) Pub. No.: US 2024/0066063 A1
(43) Pub. Date: Feb. 29, 2024

(54) COMPOSITIONS AND METHODS FOR THE TREATMENT OF CANCER USING A TGFßRII ENGINEERED T CELL THERAPY

(71) Applicant: PACT PHARMA, INC., South San Francisco, CA (US)

(72) Inventors: Charles W. TRAN, South San Francisco, CA (US); John D. Gagnon, South San Francisco, CA (US); Barbara Sennino, South San Francisco, CA (US); Stefanie Mandl-Cashman, South San Francisco, CA (US); Kyle Jacoby, South San Francisco, CA (US); William Lu, South San Francisco, CA (US); Michael Mayne Dubreuil, South San Francisco, CA (US); James S. Byers, III, South San Francisco, CA (US); Michal Mass, South San Francisco, CA (US); Alex Franzusoff, South San Francisco, CA (US)

(73) Assignee: PACT PHARMA, INC., South San Francisco, CA (US)

(21) Appl. No.: 18/319,743

(22) Filed: May 18, 2023

Related U.S. Application Data
(63) Continuation of application No. PCT/US21/60102, filed on Nov. 19, 2021.
(60) Provisional application No. 63/116,475, filed on Nov. 20, 2020.

(51) Int. Cl. A61K 35/17; A61K 39/00; A61P 35/00; C07K 16/28; C12N 5/0783; C12N 9/22; C12N 15/11

(52) U.S. Cl. CPC A61K 35/17; A61K 39/4611; A61K 39/4632; A61P 35/00; C07K 16/2815; C12N 5/0636; C12N 9/22; C12N 15/11; A61K 2239/21; A61K 2239/22

(57) ABSTRACT

Compositions comprising and methods for the treatment of cancer using a NeoTCR based cell therapy with a modified TGFβRII expression.

### CROSS-REFERENCE TO RELATED APPLICATIONS

This application is a Continuation of International Patent Application No. PCT/US2021/060102, filed on Nov. 19, 2021, which claims priority to U.S. Provisional Application No. 63/116,475, filed on Nov. 20, 2020, the contents of each of which are incorporated in their entireties, and to each of which priority is claimed.

### SEQUENCE LISTINGS

The instant application contains a Sequence Listing which has been submitted electronically in XML file format and is hereby incorporated by reference in its entirety. Said XML copy, created on May 17, 2023, is named 087520.0294.xml and is 55,745 bytes in size. The Sequence Listing, electronically filed herewith, does not extend beyond the scope of the specification and thus does not contain new matter.

### BACKGROUND OF THE INVENTION

Activation of T cells requires signaling through the T cell receptor (TCR). Activation begins when the TCR on CD4+ and CD8+ T cells binds to cognate antigen presented on antigen-presenting cells (APCs). This binding triggers a cascade of events including interactions with the MHC molecule and additional pathway signals. One of the secondary signals involved in T cell activation is provided by CD28. CD28 is found on T cells and binds to the APC. This binding initiates T cell proliferation which in turn promotes continued T cell activity.

Transforming growth factor-beta (TGFβ) is a highly conserved cytokine that has a constitutive inhibitory effect on T cell reactivity. More specifically, the TGFβ-Smad3 pathway inhibits CD28-dependent cell growth and proliferation of T cells.

NeoTCR Products described herein are T cells engineered to express a NeoTCR that are infused into patients for the treatment of immune-related diseases. Increasing the proliferative nature of the NeoTCR Cells of the NeoTCR Products can theoretically lead to improved efficacy of the NeoTCR Products because the cells will persist in the patient for a longer time post-infusion. Furthermore, T cells that have an enhanced T cell effector function and tumor cell killing activity are also beneficial for the treatment of patients. Accordingly, a NeoTCR Product that is further engineered to have enhanced T cell effector function and tumor cell killing activity is desired.

### SUMMARY OF THE INVENTION

The present disclosure provides adoptive cell therapies and methods of making the same. In certain embodiments, the present disclosure provides an immune cell comprising an exogenous T cell receptor (TCR); an exogenous CD8 receptor; and a gene disruption of a TGFβRII locus.

In certain embodiments, the present disclosure provides an immune cell comprising an exogenous polynucleotide comprising a sequence encoding an exogenous TCR and a sequence encoding an exogenous CD8 receptor; and a gene disruption of a TGFβRII locus; wherein the exogenous polynucleotide is integrated at a TRAC or TRBC locus, and wherein the sequence encoding an exogenous TCR and the sequence encoding an exogenous CD8 receptor are under control of a TRAC or TRBC promoter.

In certain embodiments, the present disclosure provides an immune cell comprising an exogenous TCR and a gene disruption of a TGFβRII locus.

In certain embodiments, the present disclosure provides an immune cell comprising an exogenous polynucleotide comprising a sequence encoding an exogenous TCR and a gene disruption of a TGFβRII locus, wherein the exogenous polynucleotide is integrated at a TRAC or TRBC locus, and wherein the sequence encoding an exogenous TCR is under control of a TRAC or TRBC promoter.

In certain embodiments, the exogenous CD8 receptor comprises a first monomer and a second monomer. In certain embodiments, the first monomer and the second monomer are the same. In certain embodiments, the first monomer and the second monomer are different. In certain embodiments, each of the first monomer and the second monomer comprise a signal peptide, an extracellular domain, a transmembrane domain, and an intracellular domain. In certain embodiments, the extracellular domain comprises a CD8α extracellular domain or a CD8β extracellular domain.

In certain embodiments, the signal peptide comprises a CD8α signal peptide or a CD8β signal peptide. In certain embodiments, the transmembrane domain comprises a CD8α transmembrane domain or a CD8β transmembrane domain. In certain embodiments, the intracellular domain is a CD8α intracellular domain, a CD8β intracellular domain, or a CD4 intracellular domain. In certain embodiments,
a) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8α signal peptide, an extracellular domain comprising a CD8α, a transmembrane domain comprising a CD8α, and an intracellular domain comprising a CD8α;
b) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8β signal peptide, an extracellular domain comprising a CD8β, a transmembrane domain comprising a CD8β, and an intracellular domain comprising a CD8β;
c) the first monomer comprises a signal peptide comprising a CD8α signal peptide, an extracellular domain comprising a CD8α, a transmembrane domain comprising a CD8α, and an intracellular domain comprising a CD8β; and the second monomer comprises a signal peptide comprising a CD8α signal peptide, an extracellular domain comprising a CD8α, a transmembrane domain comprising a CD8α, and an intracellular domain comprising a CD8β; or
d) the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments, the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments, the extracellular domain comprises
a) an amino acid sequence that is at least about 80% identical to the amino acid sequence set forth in SEQ ID NO: 10; or
b) an amino acid sequence that is at least about 80% identical to the amino acid sequence set forth in SEQ ID NO: 14.

In certain embodiments, the signal peptide comprises
a) an amino acid sequence that is at least about 80% identical to the amino acid sequence set forth in SEQ ID NO: 9; or
b) an amino acid sequence that is at least about 80% identical to the amino acid sequence set forth in SEQ ID NO: 13.

In certain embodiments, the transmembrane domain comprises
a) an amino acid sequence that is at least about 80% identical to the amino acid sequence set forth in SEQ ID NO: 11; or
b) an amino acid sequence that is at least about 80% identical to the amino acid sequence set forth in SEQ ID NO: 15.

In certain embodiments, the intracellular domain comprises
a) an amino acid sequence that is at least about 80% identical to the amino acid sequence set forth in SEQ ID NO: 12;
b) comprises an amino acid sequence that is at least about 80% identical to the amino acid sequence set forth in SEQ ID NO: 16; or
c) an amino acid sequence that is at least about 80% identical to the amino acid sequence set forth in SEQ ID NO: 17.

In certain embodiments,
a) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12;
b) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 13, an extracellular domain comprising an amino acid sequence that is at least about 80% identical to the amino acid sequence set forth in SEQ ID NO: 14, a transmembrane domain comprising an amino acid sequence that is at least about 80% identical to the amino acid sequence set forth in SEQ ID NO: 15, and an intracellular domain comprising an amino acid sequence that is at least about 80% identical to the amino acid sequence set forth in SEQ ID NO: 16;
c) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; or
d) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments, the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments,
a) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12;
b) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12; and the second monomer comprises a signal peptide comprising the amino acid sequence set forth in SEQ ID NO: 13, an extracellular domain comprising the amino acid sequence set forth in SEQ ID NO: 14, a transmembrane domain comprising the amino acid sequence set forth in SEQ ID NO: 15, and an intracellular domain comprising the amino acid sequence set forth in SEQ ID NO: 16;
c) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 16; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 16; or
d) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17.

In certain embodiments, the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17.

In certain embodiments, the gene disruption of the TGFβRII locus generates a knockout of the TGFβRII gene expression. In certain embodiments, the gene disruption of the TGFβRII locus is generated by using a CRISPR/Cas system. In certain embodiments, the CRISPR/Cas system comprises a gRNA. In certain embodiments, the gRNA comprises a nucleic acid sequence set forth in SEQ ID NOs: 1-3.

In certain embodiments, the gene disruption of the TGFβRII locus generates a knockdown of the TGFβRII gene expression.

In certain embodiments, the sequence encoding an exogenous TCR comprises a TCRα gene sequence and a TCRβ gene sequence. In certain embodiments, the sequence encoding an exogenous TCR further comprises a sequence encoding a protease cleavage site, a sequence encoding a 2A peptide, a sequence encoding a signal peptide, or a combination thereof. In certain embodiments, the exogenous polynucleotide further comprises a sequence encoding a protease cleavage site, a sequence encoding a 2A peptide, or a combination thereof.

In certain embodiments, the exogenous polynucleotide, from 5′ to 3′, comprises:
a) a first sequence encoding a 2A peptide, a sequence encoding an exogenous CD8 receptor, a first sequence encoding a protease cleavage site, a second sequence encoding a 2A peptide, a first sequence encoding a signal peptide, a TCRα gene sequence, a sequence encoding a protease cleavage site, a third sequence encoding a 2A peptide, and a TCRβ gene sequence; or
b) a first sequence encoding a 2A peptide, a sequence encoding an exogenous CD8 receptor, a first sequence encoding a protease cleavage site, a second sequence encoding a 2A peptide, a first sequence encoding a signal peptide, a TCRβ gene sequence, a sequence encoding a protease cleavage site, a third sequence encoding a 2A peptide, and a TCRα gene sequence.

In certain embodiments, the first, second, and third sequences encoding a 2A peptide are codon diverged. In certain embodiments, the first, second, and third 2A peptide comprise the amino acid sequence set forth in SEQ ID NO: 19. In certain embodiments, the first and second sequences encoding a protease cleavage site are codon diverged. In certain embodiments, the first and second sequences encoding a protease cleavage site comprise the amino acid sequence set forth in SEQ ID NOs: 4-7. In certain embodiments, the first and second sequences encoding a signal peptide are codon diverged. In certain embodiments, the first and second sequences encoding a signal peptide comprise the amino acid sequence set forth in SEQ ID NO: 35.

In certain embodiments, the immune cell further comprises a gene disruption of a TRAC locus and a TRBC locus. In certain embodiments, the exogenous TCR is a patient derived TCR. In certain embodiments, the exogenous TCR recognizes a patient derived cancer antigen. In certain embodiments, the cancer antigen is a neoantigen. In certain embodiments, the cancer antigen is a private neoantigen.

In certain embodiments, the immune cell is a patient-derived primary cell. In certain embodiments, the immune cell is a T cell, a Natural Killer T cell, a Natural Killer cell, or a lymphocyte. In certain embodiments, the immune cell is a T cell. In certain embodiments, the cell is a young T cell. In certain embodiments, the young T cell is
a) CD45RA⁺, CD62L⁺, CD28⁺, CD95⁻, CCR7⁺, and CD27⁺;
b) CD45RA⁺, CD62L⁺, CD28⁺, CD95⁺, CD27⁺, CCR7⁺; or
c) CD45RO⁺, CD62L⁺, CD28⁺, CD95⁺, CCR7⁺, CD27⁺, CD127⁺.

In certain embodiments, the exogenous TCR is a CD8-dependent TCR or a CD8-independent TCR. In certain embodiments, the immune cell further comprises a gene modification to enhance cell persistence and/or enhances memory cell differentiation. In certain embodiments, killing activity of the cell is increased between about 10% to about 500% as compared to killing activity of a cell that does not have the exogenous CD8. In certain embodiments, proliferation of the cell upon binding of the TCR to the antigen is increased between about 10% to about 500% as compared to proliferation of a cell that does not have the exogenous CD8. In certain embodiments, secretion of pro-inflammatory cytokine upon binding of the TCR to the antigen by the cell is increased between about 10% to about 500% as compared to secretion by a cell that does not have the exogenous CD8. In certain embodiments, LCK affinity of the cell is increased between about 10% to about 500% as compared to LCK affinity of a cell that does not have the exogenous CD8. In certain embodiments, persistence of the cell is increased between about 10% to about 500% as compared to persistence of a cell that does not have the exogenous CD8. In certain embodiments, tumor infiltration ability of the cell is increased between about 10% to about 500% as compared to tumor infiltration ability of a cell that does not have the exogenous CD8.

In certain embodiments, the present disclosure also provides a method of modifying a cell. In certain embodiments, the method comprises introducing into the cell a homologous recombination (HR) template nucleic acid sequence. In certain embodiments, the method further comprises recombining the HR template nucleic acid into a TRAC or TRBC locus. In certain embodiments, the method further comprises generating a gene disruption of a TGFβRII locus. In certain embodiments, the HR template comprises first and second homology arms homologous to first and second target nucleic acid sequences of a TRAC locus or a TRBC locus. In certain embodiments, the HR template further comprises a TCR gene sequence positioned between the first and second homology arms. In certain embodiments, the HR template further comprises a CD8 gene sequence positioned between the first and the second homology arms.

In certain embodiments, the method comprises introducing into the cell a homologous recombination (HR) template nucleic acid sequence. In certain embodiments, the method further comprises recombining the HR template nucleic acid into a TRAC or TRBC locus. In certain embodiments, the method further comprises generating a gene disruption of a TGFβRII locus. In certain embodiments, the HR template comprises first and second homology arms homologous to first and second target nucleic acid sequences of a TRAC locus or a TRBC locus. In certain embodiments, the HR template further comprises a TCR gene sequence positioned between the first and second homology arms.

In certain embodiments, the CD8 gene sequence encodes a first monomer and a second monomer.

In certain embodiments, each of the first monomer and the second monomer comprise a signal peptide, an extracellular domain, a transmembrane domain, and an intracellular domain.

In certain embodiments,
a) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8α signal peptide, an extracellular domain comprising a CD8α, a transmembrane domain comprising a CD8α, and an intracellular domain comprising a CD8α;
b) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8β signal peptide, an extracellular domain comprising a CD8β, a transmembrane domain comprising a CD8β, and an intracellular domain comprising a CD8β;
c) the first monomer comprises a signal peptide comprising a CD8α signal peptide, an extracellular domain comprising a CD8α, a transmembrane domain comprising a CD8α, and an intracellular domain comprising a CD8β; and the second monomer comprises a signal peptide comprising a CD8α signal peptide, an extracellular domain comprising a CD8α, a transmembrane domain comprising a CD8α, and an intracellular domain comprising a CD8β; or
d) the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments,
a) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12;
b) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 13, an extracellular domain comprising an amino acid sequence that is at least about 80% identical to the amino acid sequence set forth in SEQ ID NO: 14, a transmembrane domain comprising an amino acid sequence that is at least about 80% identical to the amino acid sequence set forth in SEQ ID NO: 15, and an intracellular domain comprising an amino acid sequence that is at least about 80% identical to the amino acid sequence set forth in SEQ ID NO: 16;
c) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; or
d) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments, the gene disruption of the TGFβRII locus generates a knockout of the TGFβRII gene expression. In certain embodiments, the gene disruption of the TGFβRII locus is generated by using a CRISPR/Cas system. In certain embodiments, the CRISPR/Cas system comprises a gRNA and a Cas9 nuclease. In certain embodiments, the gRNA comprises a nucleic acid sequence set forth in SEQ ID NOs: 1-3. In certain embodiments, the TCR gene sequence comprises a TCRα gene sequence and a TCRβ gene sequence.

In certain embodiments, TCR gene sequence further comprises a sequence encoding a protease cleavage site, a sequence encoding a 2A peptide, a sequence encoding a signal peptide, or a combination thereof. In certain embodiments, the HR template further comprises a sequence encoding a protease cleavage site, a sequence encoding a 2A peptide, or a combination thereof. In certain embodiments, the HR template, from 5′ to 3′, comprises:
a) a first sequence encoding a 2A peptide, a sequence encoding an exogenous CD8 receptor, a first sequence encoding a protease cleavage site, a second sequence encoding a 2A peptide, a first sequence encoding a signal peptide, a TCRα gene sequence, a sequence encoding a protease cleavage site, a third sequence encoding a 2A peptide, and a TCRβ gene sequence; or
b) a first sequence encoding a 2A peptide, a sequence encoding an exogenous CD8 receptor, a first sequence encoding a protease cleavage site, a second sequence encoding a 2A peptide, a first sequence encoding a signal peptide, a TCRβ gene sequence, a sequence encoding a protease cleavage site, a third sequence encoding a 2A peptide, and a TCRα gene sequence.

In certain embodiments,
a) the first, second, and third sequences encoding a 2A peptide are codon diverged;
b) the first and second sequences encoding a protease cleavage site are codon diverged; and
c) the first and second sequences encoding a signal peptide are codon diverged.

In certain embodiments,
a) the first, second, and third 2A peptide comprise the amino acid sequence set forth in SEQ ID NO: 19;
b) the first and second sequences encoding a protease cleavage site comprise the amino acid sequence set forth in SEQ ID NOs: 4-7; and
c) the first and second sequences encoding a signal peptide comprise the amino acid sequence set forth in SEQ ID NO: 35.

In certain embodiments, the first and second homology arms of the HR template are each from about 300 bases to about 2,000 bases in length. In certain embodiments, the method further comprises generating a gene disruption of a TRAC locus. In certain embodiments, the method further comprises generating a gene disruption of a TRBC locus.

In certain embodiments, the HR template is non-viral. In certain embodiments, the HR template is a circular DNA. In certain embodiments, the HR template is a linear DNA. In certain embodiments, the introducing occurs via electroporation. In certain embodiments, the TCR gene sequence is a patient derived sequence. In certain embodiments, the TCR gene sequence encodes a TCR recognizing a patient derived cancer antigen. In certain embodiments, the cancer antigen is a neoantigen. In certain embodiments, the cancer antigen is a private neoantigen.

In certain embodiments, the immune cell is a patient-derived primary cell. In certain embodiments, the immune cell is a T cell, a Natural Killer T cell, a Natural Killer cells, or a lymphocyte. In certain embodiments, the cell is a T cell.

In certain embodiments, the method further comprises culturing the cell in the presence of at least one cytokine. In certain embodiments, the at least one cytokine comprises IL2, IL7, IL15, or a combination thereof. In certain embodiments, the at least one cytokine comprises IL7 and IL15.

In certain embodiments, the present disclosure provides a cell modified by the method disclosed herein.

In certain embodiments, the present disclosure provides an immune cell comprising an exogenous T cell receptor (TCR) and a dominant negative TGFβRII (dnTGFβRII). In certain embodiments, the present disclosure provides an immune cell comprising an exogenous polynucleotide comprising a sequence encoding an exogenous T cell receptor (TCR) and a sequence encoding a dominant negative TGFβRII (dnTGFβRII). In certain embodiments, the exogenous polynucleotide is integrated at a TRAC or TRBC locus. In certain embodiments, the sequence encoding an exogenous TCR is under control of a TRAC or TRBC promoter, and wherein the sequence encoding a dnTGFβRII is under control of an exogenous promoter. In certain embodiments, the exogenous polynucleotide further comprises an exogenous enhancer, an insulator, a pause element, a Woodchuck Hepatitis Virus Posttranscriptional Regulatory Element (WPRE), a poly-A sequence, or a combination thereof.

In certain embodiments, the present disclosure further provides a composition comprising the cell disclosed herein. In certain embodiments, the composition is a pharmaceutical composition comprising a pharmaceutically acceptable excipient. In certain embodiments, the composition further comprises a crystalloid solution, a cryopreservation agent, serum albumin, or a combination thereof. In certain embodiments, the composition further comprises Plasma-Lyte A, CryoStor CS10, human serum albumin, or a combination thereof.

In certain embodiments, the present disclosure further provides the cell or the composition disclosed herein for use in treating cancer in a subject. In certain embodiments, the cancer is a solid tumor or a liquid tumor. In certain embodiments, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments, the liquid tumor is selected from the group consisting of follicular lymphoma, leukemia, and multiple myeloma.

In certain embodiments, the present disclosure provides a method of treating cancer in a subject in need thereof. In certain embodiments, the method comprises administering a therapeutically effective amount of the cell or the composition disclosed herein. In certain embodiments, prior to administering the therapeutically effective amount of cells, a non-myeloablative lymphodepletion regimen is administered to the subject.

In certain embodiments, the cancer is a solid tumor. In certain embodiments, the cancer is liquid tumor. In certain embodiments, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV⁺ cancer, esophageal cancer, thyroid cancer, gastric cancer, hepatocellular cancer, cholangiocarcinomas, renal cell cancers, testicular cancer, sarcomas, and colorectal cancer. In certain embodiments, the liquid tumor is selected from the group consisting of follicular lymphoma, leukemia, and multiple myeloma.

In certain embodiments, the present disclosure provides a kit comprising the cell, reagents for performing the method, or the composition disclosed herein. In certain embodiments, the kit further comprises written instructions for treating a cancer.

### BRIEF DESCRIPTION OF THE DRAWINGS

FIGS. 1A-1C. FIGS. 1A-1C show an example of a NeoE TCR cassette and gene editing methods that can be used to make NeoTCR Products. FIG. 1A shows a schematic representing the general targeting strategy used for integrating neoantigen-specific TCR constructs (NeoTCRs) into the TCRα locus. FIGS. 1B and 1C show a neoantigen-specific TCR construct design used for integrating a NeoTCR into the TCRα locus wherein the cassette is shown with signal sequences (“SS”), protease cleavage sites (“P”), and 2A peptides (“2A”). FIG. 1B shows a target TCRα locus (endogenous TRAC, top panel) and its CRISPR Cas9 target site (horizontal stripes, cleavage site designated by the arrow), and the circular plasmid HR template (bottom panel) with the polynucleotide encoding the NeoTCR, which is located between left and right homology arms (“LHA” and “RHA” respectively) prior to integration. FIG. 1C shows the integrated NeoTCR in the TCRα locus (top panel), the transcribed and spliced NeoTCR mRNA (middle panel), and translation and processing of the expressed NeoTCR (bottom panel).

FIG. 2. FIG. 2 shows the gene-editing approaches that are used to inhibit the TGFβ pathway.

FIGS. 3A and 3B. FIGS. 3A and 3B depict a dnTGFβRII Construct.

FIG. 4. FIG. 4 provides a high-level diagram of the knock-out and knock-in at the endogenous TCR locus and the knock-out of the TGFβRII gene accomplished by the gene-editing technology described herein. Table 1 provides three exemplary gRNAs for the knockout of the TGFβRII gene.

FIGS. 5A and 5B. FIG. 5A shows the gene-editing efficiency of the insertion of a NeoTCR (TCR089) alone compared to the insertion of a NeoTCR (TCR089) and a dominant negative TGFβRII (dnTGFβRII), both under the control of the TCR promoter, in CD8+ and CD4+ cells. As shown, the insertion of the dnTGFβRII did not affect the NeoTCR expression. FIG. 5B shows that the insertion of the dnTGFβRII did not reduce pSMAD2/3 following TGFβI treatment.

FIG. 6. FIG. 6 shows that TGFβRII can be knocked down using gRNAs and that the dnTGFβRII can be successfully expressed on NeoTCR Cells. Accordingly, expression and knockdown efficiency are not limiting factors.

FIGS. 7A and 7B. FIG. 7A shows that TGFβRII knockouts inhibit TGFβ-induced SMAD2/3 phosphorylation in both CD8+ and CD4+ T cells. FIG. 7B shows that TGFβRII knockouts enhance CD8+ and CD4+ T cell IFNγ production (*p<0.05, **p<0.01, ***p<0.001, ****p<0.0001 Two-Way ANOVA, Tukey test).

FIGS. 8A and 8B. FIG. 8A shows FACS assays gated on CD8+ and Dextramer+ (i.e., NeoTCR+) cells and shows that the knockout of TGFRβII enhances CD8+ T cell IFNγ production. FIG. 8B are FACS assays gated on CD4+ and Dextramer+ (i.e., NeoTCR+) cells and shows that the knockout of TGFRβII enhances CD4+ T cell IFNγ production.

FIGS. 9A and 9B. FIGS. 9A and 9B show that the knockout of TGFβRII restores T cell proliferation in the presence of TGFβ1 in CD4+ (FIG. 9A) and CD8+ (FIG. 9B) cells.

FIGS. 10A and 10B. FIGS. 10A and 10B shows that TGFβ inhibits CD25 upregulating in CD4+ T cells (FIG. 10A) but not in CD8+ T cells (FIG. 10B). As shown in FIG. 10A, the gRNA knockouts were able to rescue the CD4+ T cells from the effects of TGFβ but the expression of the dnTGFβRII was not able to achieve the same rescue efficiency.

FIG. 11. FIG. 11 shows that the ability of NeoTCR Cells to kill a matched tumor line is not affected by the presence of TGFβ1. The NeoTCR Cells shown in this figure are 1) the NeoTCR Cell expressing only TCR089, 2) the NeoTCR Cell expressing TCR089 and a dnTGFβRII, and 3) the NeoTCR Cell expressing TCR089 in conjunction with the TGFβRII being knocked out by any one of gRNA1, gRNA2, or gRNA3. This figure shows the results of the NeoTCRto Tumor Cell E:T ratio of 2:1 (experiments with E:T ratios of 1:1 and 1:2 were also performed with similar results).

FIGS. 12A and 12B. FIGS. 12A and 12B (gated on CD8+ NeoTCR Cells) shows that TGFβ reduces the capacity of NeoTCR Cells without a TGFβRII knockout to kill. Conversely, TGFβRII knockout NeoTCR Cells were able to retain their killing ability as demonstrated in a stimulation assay that tracks sustained perforin (FIG. 12B) and granzyme B (FIG. 12A) expression in the presence of TGFβ.

FIGS. 13A and 13B. FIG. 13A shows a diagram of Format 1 of the secondary promoter constructs used to express a broad selection of knock in (KI) targets. The aim of Format 1 constructs is to decouple the NeoTCR expression from a secondary KI gene (i.e., a payload (e.g., dnTGFβRII)). The Format 1 constructs are modular such that the payload (e.g., dnTGFβRII), the TCR, the promoter, and insulators can be changed to suit the needs of the genetic engineering goals. As shown, the architecture of Format 1 construct comprises a left homology arm, a linker sequence, a 2A sequence, a signal sequence, a full length TCR beta gene, a protease cleavage sequence, a linker, a 2A sequence, a signal sequence, a full length TCR alpha gene, a poly-A sequence, an insulator sequence, a promoter region, an optional Kozak sequence, a payload (e.g., dnTGFβRII) of interest, and a right homology arm; all of which are retained within a backbone. FIG. 13B shows an alternate modified version of Format 1 wherein a poly-A sequence is incorporated after the payload (e.g., dnTGFβRII). The abbreviations for the elements of Format 1 used in FIGS. 13A and 13B are 2A (2A sequence), SS (signal sequence), P (protease cleavage site), I (insulator).

FIGS. 14A and 14B. FIG. 14A shows a diagram of an example of Format 1 of the secondary promoter constructs used to express a broad selection of KI targets. As shown, this example of a Format 1 construct comprises a left homology arm, a GSG linker, a P2A sequence, an HGH signal sequence, a full length TCR beta gene, a furin cleavage sequence, a GSG linker, a P2A sequence, an HGH signal sequence, a full length TCR alpha gene, a poly-A sequence, an insulator sequence, a promoter region, a Kozak sequence, a payload (e.g., dnTGFβRII) of interest, and a right homology arm; all of which are retained within a backbone. FIG. 14B shows an alternate example of Format 1 wherein a poly-A sequence is incorporated after the payload (e.g., dnTGFβRII). The abbreviations for the Insulator used in Format 1 used in FIGS. 14A and 14B is “I”.

FIGS. 15A-15C. FIG. 5A shows a diagram of Format 2 of the secondary promoter constructs used to express abroad selection of KI targets. The aim of Format 2 constructs is to decouple the NeoTCR expression from a secondary KI gene (i.e., a payload (e.g., dnTGFβRII)). The Format 2 constructs are modular such that the payload (e.g., dnTGFβRII), the TCR, the promoter, and insulators can be changed to suit the needs of the genetic engineering goals. As shown, the architecture of Format 2 construct comprises a left homology arm, a linker sequence, a 2A sequence, a signal sequence, a full length TCR beta gene, a protease cleavage sequence, a linker sequence, a 2A sequence, a signal sequence, a full length TCR alpha gene, a poly-A sequence, an insulator sequence, a promoter region, a Kozak sequence (optional), a payload (e.g., dnTGFβRII) of interest, a WPRE, a poly-A sequence, and a right homology arm; all of which are retained within a backbone. FIG. 15B shows an alternate version of Format 2 wherein the post transcriptional regulatory element is not included and instead an insulator is incorporated following the second poly-A sequence. FIG. 15C shows an alternate version of Format 2 wherein the post transcriptional regulatory element is not included and no insulator is incorporated following the second poly-A sequence. The abbreviations for the elements of Format 2 used in FIGS. 15A-15C are 2A (2A sequence), SS (signal sequence), P (protease cleavage site), I (insulator).

FIGS. 16A-16C. FIG. 16A shows a diagram of an example of Format 2 of the secondary promoter constructs used to express a broad selection of knock in (KI) targets. As shown, the architecture of this example of Format 2 constructs comprise a left homology arm; GSG linker; P2A sequence; HGH signal sequence; full length TCR beta gene; Furin cleavage sequence; GSG linker; P2A sequence; HGH signal sequence; full length TCR alpha gene; poly-A sequence; insulator sequence; promoter region; Kozak sequence; payload (e.g., dnTGFβRII) of interest; WPRE; poly-A sequence; right homology arm; all of which are retained within a backbone. FIG. 16B shows an example of one of the alternate versions of Format 2 wherein the post transcriptional regulatory element is not included and instead an insulator is incorporated following the second poly-A sequence. FIG. 16C shows an example of one of the alternate versions of Format 2 wherein the post transcriptional regulatory element is not included and no insulator is incorporated following the second poly-A sequence. The abbreviations for the Insulator used in Format 1 used in FIGS. 16A-16C is “I”.

FIG. 17. FIG. 17 shows a diagram of Format 3 of the secondary promoter constructs used to express a broad selection of KI targets. The aim of Format 3 constructs is to decouple the NeoTCR expression from a secondary KI gene (i.e., a payload (e.g., dnTGFβRII)). The Format 3 constructs are modular such that the payload (e.g., dnTGFβRII), the TCR, the promoter, and insulators can be changed to suit the needs of the genetic engineering goals. As shown, the architecture of Format 3 construct comprises a left homology arm, a linker sequence, a 2A sequence, a signal sequence, a full length TCR beta gene, a protease cleavage sequence, a linker sequence, a 2A sequence, a signal sequence, a full length TCR alpha gene, a poly-A sequence, a WPRE, a payload (e.g., dnTGFβRII) of interest, a Kozak sequence (optional), a promoter region, and a right homology arm; all of which are retained within a backbone. The abbreviations for the elements of Format 1 used in FIG. 17 are 2A (2A sequence), SS (signal sequence), P (protease cleavage site), I (insulator).

FIG. 18. FIG. 18 shows a diagram of an example of Format 3 of the secondary promoter constructs used to express a broad selection of KI targets. The aim of Format 3 constructs is to decouple the NeoTCR expression from a secondary KI gene (i.e., a payload (e.g., dnTGFβRII)). The Format 3 constructs are modular such that the payload (e.g., dnTGFβRII), the TCR, the promoter, and insulators can be changed to suit the needs of the genetic engineering goals. As shown, the architecture of this example of Format 3 constructs comprise a left homology arm, a GSG linker, a P2A sequence, an HGH signal sequence, a full length TCR beta gene, a Furin cleavage sequence, a GSG linker, a P2A sequence, an HGH signal sequence, a full length TCR alpha gene, a poly-A sequence, a WPRE, a payload (e.g., dnTGFβRII) of interest, a Kozak sequence, a promoter region, and a right homology arm; all of which are retained within a backbone. The abbreviation for the Insulator used in Format 1 depicted in FIG. 18 is “I”.

FIG. 19. FIG. 19 shows a diagram of Format 4 of the secondary promoter constructs used to express a broad selection of KI targets. The aim of Format 4 is to control the expression of the payload (e.g., dnTGFβRII) and the TCR off of the endogenous TRAC or TRBC promoter. Unlike Formats 1-3 that only have the TCR controlled by the TRAC or TRBC promoter and have the payload (e.g., dnTGFβRII)s controlled by a secondary promoter, Format 4 controls both the TCR and the payload (e.g., dnTGFβRII) off of the single, endogenous TRAC or TRBC promoter.

FIGS. 20A-20D. FIGS. 20A-20D show the circular plasmids used to encode CD8 constructs 1, 2, 3, and 4. FIG. 20A shows CD8 Construct 1 used to produce the CD8 Product 1. FIG. 20B shows CD8 Construct 2 used to produce the CD8 Product 2. FIG. 20C shows CD8 Construct 3 used to produce the CD8 Product 3. FIG. 20D shows CD8 Construct 4 used to produce the CD8 Product 4. As shown in FIGS. 20A-20D, SS stands for a signal sequence. The SS may be HGH; however other signal sequences may be used as needed for appropriate trafficking. As shown in FIGS. 20A-20D, P stands for a protease cleavage site. The P may be Furin; however other protease cleavage sites may be used as appropriate to provide the cleavage action described herein. As shown in FIGS. 20A-20D, 2A stands for the 2A peptide. The 2A may be the P2A peptide; however, other 2A peptides may be used.

FIG. 21. FIGS. 21A-21D show the transcription/splicing and translation processing of each CD8 Construct 1, CD8 Construct 2, CD8 Construct 3, and CD8 Construct 4. As shown in FIGS. 21A-21D, SS stands for a signal sequence. The SS may be HGH; however other signal sequences may be used as needed for appropriate trafficking. As shown in FIGS. 21A-21D, P stands for a protease cleavage site. The P may be Furin; however other protease cleavage sites may be used as appropriate to provide the cleavage action described herein. As shown in FIGS. 21A-21D, 2A stands for the 2A peptide. The 2A may be the P2A peptide; however, other 2A peptides may be used.

FIGS. 22A and 22B. FIG. 22A shows translated products of CD8 Construct 1 and CD8 Construct 2. FIG. 22B shows the translated products of CD8 Construct 3 and CD8 Construct 4.

FIGS. 23A-23H. FIG. 23A shows representative flow cytometry histograms illustrating the proliferation of unstimulated and stimulated CD4 T cells from a first donor. FIG. 23B shows representative flow cytometry histograms illustrating the proliferation of unstimulated and stimulated CD8 T cells from a first donor. FIG. 23C shows the expansion index of stimulated (10 ng/ml) CD4 T cells. FIG. 23D shows the expansion index of stimulated (100 ng/ml) CD4 T cells. FIG. 23E shows the expansion index of unstimulated CD4 T cells. FIG. 23F shows the expansion index of stimulated (10 ng/ml) CD8 T cells. FIG. 23G shows the expansion index of stimulated (100 ng/ml) CD8 T cells. FIG. 23H shows the expansion index of unstimulated CD8 T cells. “TCR089”: T cells expressing exogenous TCR089. “+CD8a-4ID”: T cells expressing exogenous TCR089 and a chimeric CD8 receptor comprising a CD8 extracellular domain and a CD4 intracellular domain. “+TGFBRII KO”: T cells expressing exogenous TCR089 and knockout for TGFBRII; “+CD8a-4ID+KO”: T cells knockout for TGFBRII and expressing exogenous TCR089 and a chimeric CD8 receptor comprising a CD8 extracellular domain and a CD4 intracellular domain.

FIGS. 24A-24H. FIG. 24A shows representative flow cytometry histograms illustrating the proliferation of unstimulated and stimulated CD4 T cells from a second donor. FIG. 24B shows representative flow cytometry histograms illustrating the proliferation of unstimulated and stimulated CD8 T cells from a first donor. FIG. 24C shows the expansion index of stimulated (10 ng/ml) CD4 T cells. FIG. 24D shows the expansion index of stimulated (100 ng/ml) CD4 T cells. FIG. 24E shows the expansion index of unstimulated CD4 T cells. FIG. 24F shows the expansion index of stimulated (10 ng/ml) CD8 T cells. FIG. 24G shows the expansion index of stimulated (100 ng/ml) CD8 T cells. FIG. 24H shows the expansion index of unstimulated CD8 T cells. “TCR097”: T cells expressing exogenous TCR097. “+CD8a-4ID”: T cells expressing exogenous TCR097 and a chimeric CD8 receptor comprising a CD8 extracellular domain and a CD4 intracellular domain. “+TGFBRII KO”: T cells expressing exogenous TCR097 and knockout for TGFBRII; “+CD8a-4ID+KO”: T cells knockout for TGFBRII and expressing exogenous TCR097 and a chimeric CD8 receptor comprising a CD8 extracellular domain and a CD4 intracellular domain.

FIGS. 25A-25H show activation of T cells disclosed herein. FIG. 25A shows CD25 expression of stimulated (10 mg/ml) CD4 T cell from first donor. FIG. 25B shows CD25 expression of stimulated (100 mg/ml) CD4 T cell from first donor. FIG. 25C shows CD25 expression of stimulated (10 mg/ml) CD8 T cell from first donor. FIG. 25D shows CD25 expression of stimulated (100 mg/ml) CD8 T cell from first donor. FIG. 25E shows CD25 expression of stimulated (10 mg/ml) CD4 T cell from second donor. FIG. 25F shows CD25 expression of stimulated (100 mg/ml) CD4 T cell from second donor. FIG. 25G shows CD25 expression of stimulated (10 mg/ml) CD8 T cell from second donor. FIG. 25H shows CD25 expression of stimulated (100 mg/ml) CD8 T cell from second donor.

FIGS. 26A-25F show intracellular cytokine staining after stimulation. FIG. 26A shows IFNγ intracellular staining of CD4 and CD8 T cells from first donor after stimulation with neoantigen (neoE-HLA) and incubated with or without TGFβ. FIG. 26B shows TNFα intracellular staining of CD4 and CD8 T cells from first donor after stimulation with neoantigen (neoE-HLA) and incubated with or without TGFβ. FIG. 25C shows IFNγ and TNFα intracellular staining of CD4 and CD8 T cells from first donor after stimulation with neoantigen (neoE-HLA) without TGFβ. FIG. 26D shows IFNγ intracellular staining of CD4 and CD8 T cells from second donor after stimulation with neoantigen (neoE-HLA) and incubated with or without TGFβ. FIG. 26E shows TNFα intracellular staining of CD4 and CD8 T cells from second donor after stimulation with neoantigen (neoE-HLA) and incubated with or without TGFβ. FIG. 25F shows IFNγ and TNFα intracellular staining of CD4 and CD8 T cells from second donor after stimulation with neoantigen (neoE-HLA) without TGFβ.

FIGS. 27A-27H show activation markers after stimulation. FIG. 27A shows 4-1BB in CD4 and CD8 T cells from first donor after stimulation with neoantigen (neoE-HLA) and incubated with or without TGFβ. FIG. 27B shows OX40 expression in CD4 and CD8 T cells from first donor after stimulation with neoantigen (neoE-HLA) and incubated with or without TGFβ. FIG. 27C shows CD40L expression in CD4 and CD8 T cells from first donor after stimulation with neoantigen (neoE-HLA) and incubated with or without TGFβ. FIG. 27D shows expression of 4-1BB, OX40, and CD40L of CD4 and CD8 T cells from first donor after stimulation with neoantigen (neoE-HLA) without TGFβ. FIG. 27E shows 4-1BB expression in CD4 and CD8 T cells from second donor after stimulation with neoantigen (neoE-HLA) and incubated with or without TGFβ. FIG. 27F shows OX40 expression in CD4 and CD8 T cells from second donor after stimulation with neoantigen (neoE-HLA) and incubated with or without TGFβ. FIG. 27G shows CD40L expression in CD4 and CD8 T cells from second donor after stimulation with neoantigen (neoE-HLA) and incubated with or without TGFβ. FIG. 27H shows expression of 4-1BB, OX40, and CD40L of CD4 and CD8 T cells from second donor after stimulation with neoantigen (neoE-HLA) without TGFβ.

FIGS. 28A-28C show the effect of different RNP ratios on the proliferation and activation of cells. FIG. 28A shows the gating strategy. FIG. 28B shows TGFBR2 expression in CD4 T cells with different RNPs. FIG. 28C shows TGFBR2 expression in CD8 T cells with different RNPs.

FIGS. 29A-29H show the effect of different RNP ratios on the proliferation and activation of cells. FIG. 29A shows representative flow cytometry histograms illustrating the proliferation and activation (CD25) of CD4 TGFBRII knockout T cells stimulated with 10 ng/ml comPACT and incubated with or without TGFβ. FIG. 29B shows representative flow cytometry histograms illustrating the proliferation and activation (CD25) of CD8 TGFBRII knockout T cells stimulated with 10 ng/ml comPACT and incubated with or without TGFβ. FIG. 29C shows histograms quantifying experiments of FIG. 29A. FIG. 29D shows histograms quantifying experiments of FIG. 29B. FIG. 29E shows representative flow cytometry histograms illustrating the proliferation and activation (CD25) of CD4 TGFBRII knockout T cells stimulated with 100 ng/ml comPACT and incubated with or without TGFβ. FIG. 29F shows representative flow cytometry histograms illustrating the proliferation and activation (CD25) of CD8 TGFBRII knockout T cells stimulated with 100 ng/ml comPACT and incubated with or without TGFβ. FIG. 29G shows histograms quantifying experiments of FIG. 29D. FIG. 29H shows histograms quantifying experiments of FIG. 29E.

### DETAILED DESCRIPTION

The present disclosure provides adoptive cell therapies comprising a NeoTCR and additional genomic modifications (e.g., knockout of the TGFβRII locus) and having enhanced activity and efficacy against tumor cells. The present disclosure is based, in part, on the ability of the inventors to improve the activity and efficacy of these cells against tumor microenvironment enriched with immunosuppressive molecules (e.g., TGFβ). These cells showed potentiated and improved activity (e.g., cytotoxicity, cell proliferation, and/or cell persistence). The present disclosure also provides methods for producing the cells, and compositions disclosed herein. Finally, the present disclosure provides methods of using such cells and composition for treating and/or preventing cancer.

Non-limiting embodiments of the present disclosure are described by the present description and examples. For purposes of clarity of disclosure and not by way of limitation, the detailed description is divided into the following subsections:
1. Definitions;
2. NeoTCR Products;
3. Methods of Treatment;
4. Articles of Manufacture;
5. Therapeutic Compositions and Methods of Manufacturing;
6. Kits; and
7. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes,” “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

The term “antibody” as used herein is used in the broadest sense and encompasses various antibody structures, including but not limited to monoclonal antibodies, polyclonal antibodies, multispecific antibodies (e.g., bispecific, and tri-specific antibodies), and antibody fragments (e.g., bis-Fabs) so long as they exhibit the desired antigen-binding activity. “Antibody Fragment” as used herein refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv); and multispecific antibodies formed from antibody fragments.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

As used herein, “sequence identity” or “identity” (in the context of two nucleic acid or polypeptide sequences) refers to the residues in the two sequences that are the same when aligned for maximum correspondence over a specified comparison window. When the percentage of sequence identity is used about proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art.

As used herein, “percentage of sequence identity” refers to the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for the optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Methods of alignment of sequences for comparison include, without any limitation, the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877. Computer implementations of these mathematical algorithms include, without any limitation, CLUSTAL, CLUSTALW, CLUSTALOMEGA, ALIGN, ALIGN PLUS, GAP, BESTFIT, BLAST, FASTA, TFASTA, BLASTN, BLASTX, BLASTP, TBLASTN, and TBLASTX.

A “conservative substitution” or a “conservative amino acid,” refers to the substitution of amino acid with a chemically or functionally similar amino acid. Conservative substitution tables providing similar amino acids are well known in the art. In certain embodiments, acidic amino acids D and E are conservative substitutions for one another; basic amino acids K, R, and H are conservative substitutions for one another; hydrophilic uncharged amino acids S, T, N. and Q are conservative substitutions for one another; aliphatic uncharged amino acids G, A, V, L, and I are conservative substitutions for one another; non-polar uncharged amino acids C, M, and P are conservative substitutions for one another; aromatic amino acids F, Y, and W are conservative substitutions for one another; A, S, and T are conservative substitutions for one another; D and E are conservative substitutions for one another; N and Q are conservative substitutions for one another; R and K are conservative substitutions for one another; I, L, and M are conservative substitutions for one another; F, Y, and W are conservative substitutions for one another; A and G are conservative substitutions for one another; D and E are conservative substitutions for one another; N and Q are conservative substitutions for one another; R, K and H are conservative substitutions for one another; I, L, M, and V are conservative substitutions for one another; F, Y and W are conservative substitutions for one another; S and T are conservative substitutions for one another; and C and M are conservative substitutions for one another. Additional conservative substitutions may be found, for example, in Creighton, Proteins: Structures and Molecular Properties 2nd ed. (1993) W. H. Freeman & Co., New York, NY.

“Secondary Promoter Construct,” as used herein, refers to a construct comprising elements to express a NeoTCR and elements to express a payload (e.g., dnTGFβRII). Formats 1-4, along with the variations thereof described herein, are examples of Secondary Promoter Constructs. Additional examples of secondary promoter constructs encompassed by the present disclosure can be found in International Patent Application No. PCT/US21/56737, the content of which is incorporated by reference in its entirety.

“Payload” as used herein refers to a second or more gene (e.g., dnTGFβRII), in addition to the NeoTCR, to be knocked into an immune cell using gene editing methods described herein. Examples of Payloads include but are not limited to the one disclosed in International Patent Application No. PCT/US21/56737, the content of which is incorporated by reference in its entirety.

“Promoter region” or “promoter” as used herein means the region of the construct that encodes a promoter that controls the expression of the payload (e.g., dnTGFβRII) in the secondary promoter constructs.

As used herein, the term “enhancer” refers to a DNA control element that enhances the levels of expression of the gene when a specific transcription factor is bound. Unlike a promoter, an enhancer does not stimulate the expression of the gene on its own. Enhancers are frequently found in the upstream (5′) region of the gene.

As used herein, a “STOP codon” or “termination codon” is a nucleotide triplet within a messenger RNA that signals the termination of the translation process of a protein. Most codons in messenger RNA correspond to the addition of an amino acid to a growing polypeptide chain, which can ultimately become a protein; stop codons signal the termination of this process by binding release factors, which cause the ribosomal subunits to disassociate, releasing the amino acid chain.

“WPRE” as used herein refers to a woodchuck hepatitis virus post-transcriptional regulatory element that increases transgene expression.

“Kozak” or “Kozak sequence” as used herein refers to a nucleic acid motif that functions as the protein translation initiation site in certain eukaryotic mRNA transcripts.

“Insulator” or “transcriptional insulator,” as used herein, refer to a class of DNA sequence elements that possess a common ability to protect genes from inappropriate signals emanating from their surrounding environment. Insulators can be used to restrict the interaction of enhancers or silencers on promoters in a gene expression system. Insulators can set boundaries on the actions of enhancer and silencer elements and so partition the eukaryotic genome into regulatory domains. Physiologically, the transcriptional repressor CTCF (CCCTC-binding factor) binds through multiple zinc fingers (of which it has eleven) to a range of unrelated DNA sequences and functions as a transcriptional insulator, repressor, or activator, depending on the context of the binding site.

“Signal sequence” as used herein is a peptide that can be included at the N-terminus of a newly synthesized protein for the purpose of trafficking the newly synthesized protein to its intended and/or engineered location inside or outside of the cell.

As used herein, the term “gene disruption” refers to any modification of the genome or transcriptome of a cell capable of altering the physiological expression level of a gene or protein. A gene disruption includes mutations, deletions, insertions, or combinations thereof. In certain embodiments, the mutation can be a missense mutation, a nonsense mutation, or a combination thereof. In certain embodiments, the deletion can be a non-frameshift deletion, a frameshift deletion, or a combination thereof. In certain embodiments, the insertion can be a non-frameshift insertion, a frameshift insertion, or a combination thereof “TCR,” as used herein, means T cell receptor.

“NeoTCR” and “NeoE TCR,” as used herein, mean a neoepitope-specific T cell receptor that is introduced into a T cell, e.g., by gene editing methods. As used herein, the term “exogenous TCR gene sequence” refers to a NeoTCR gene sequence.

“NeoTCR cells,” as used herein, means one or more cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR cells” and “NeoTCR-P1 T cells” and “NeoTCR-P1 cells” are used interchangeably herein. In certain embodiments, TGFβRII Cells, dnTGFβRII Cells, and CD8-TGFβRII Cells are each a type of NeoTCR cells.

“NeoTCR Product,” as used herein, means a pharmaceutical formulation comprising one or more NeoTCR cells. NeoTCR Product consists of autologous precision genome-engineered CD8+ and CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states. The NeoTCR Product manufactured for each patient represents a defined dose of autologous CD8+ and/or CD4+ T cells that are precision genome engineered to express a single neoE-specific TCR cloned from neoE-specific CD8+ T cells individually isolated from the peripheral blood of that same patient. In certain embodiments, TGFβRII Products, dnTGFβRII Products, and CD8-TGFβRII Products are each a type of NeoTCR Product.

“NeoTCR Viral Product” as used herein has the same definition of NeoTCR Product except that the genome engineering is performed using viral mediated methods.

As used herein, “TGFβRII” refers to Transforming Growth Factor Beta Receptor 2.

“dnTGFβRII,” as used herein, means a dominant negative form of TGFβRII.

“dnTGFβRII Construct,” as used herein, means a Secondary Promoter Construct wherein the Payload (e.g., dnTGFβRII) is a dominant negative form of the TGFβRII (e.g., dnTGFβRII). A dnTGFβRII includes mutations that alter the TGFβRII so that the receptor acts antagonistically to the wild-type receptor. These mutations can include mutations that result in an altered molecular function (e.g., inactive receptor) and are characterized by a dominant or semi-dominant phenotype.

“dnTGFβRII Cells,” as used herein, means one or more cells precision engineered to express a NeoTCR and a dnTGFβRII.

“dnTGFβRII Product,” as used herein, means a product comprising dnTGFβRII Cells.

“TGFβRII Cells,” as used herein, means one or more cells precision engineered to express a NeoTCR and including knockout the endogenous expression of TGFβRII. As used in this definition, “knockout” refers to a complete or substantial knockout of the TGFβRII taking into account the fact that a certain amount TGFβRII expression may persist due to incomplete knockout.

“TGFβRII Product,” as used herein, means a product comprising TGFβRII Cells.

As used herein, “CD8” is a cell surface glycoprotein found on most cytotoxic T lymphocytes that mediates efficient cell-cell interactions within the immune system. The CD8 antigen acts as a coreceptor with the T-cell receptor on the T lymphocyte to recognize antigens displayed by an antigen presenting cell in the context of class I MHC molecules.

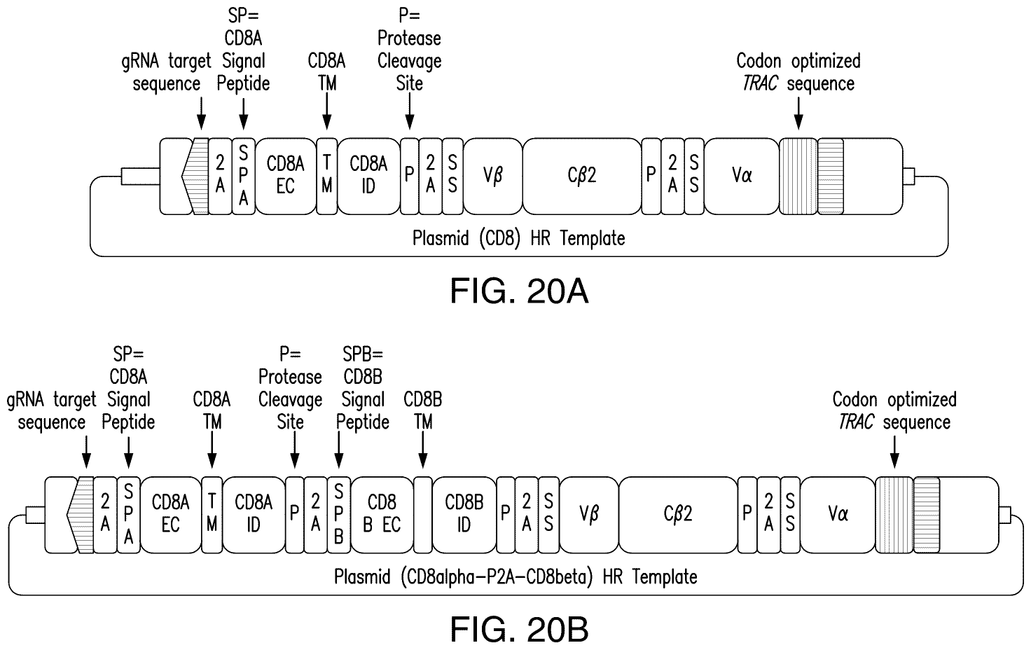

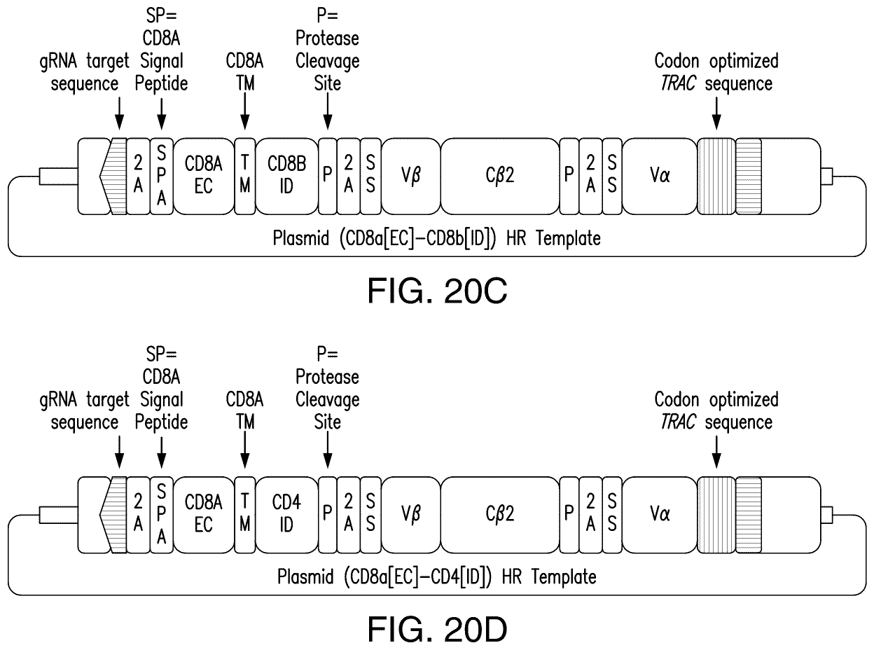

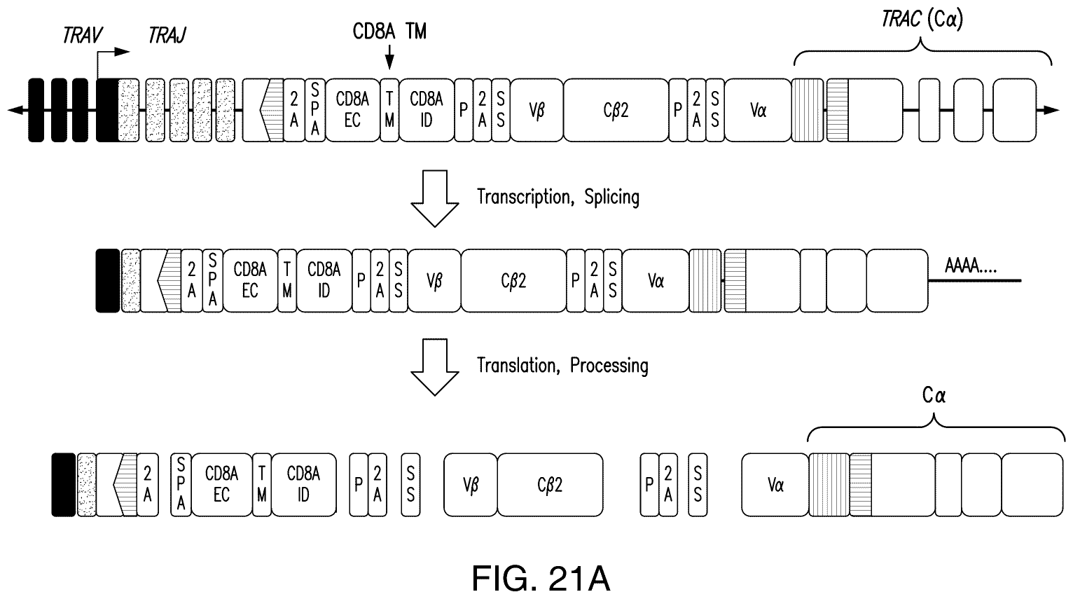

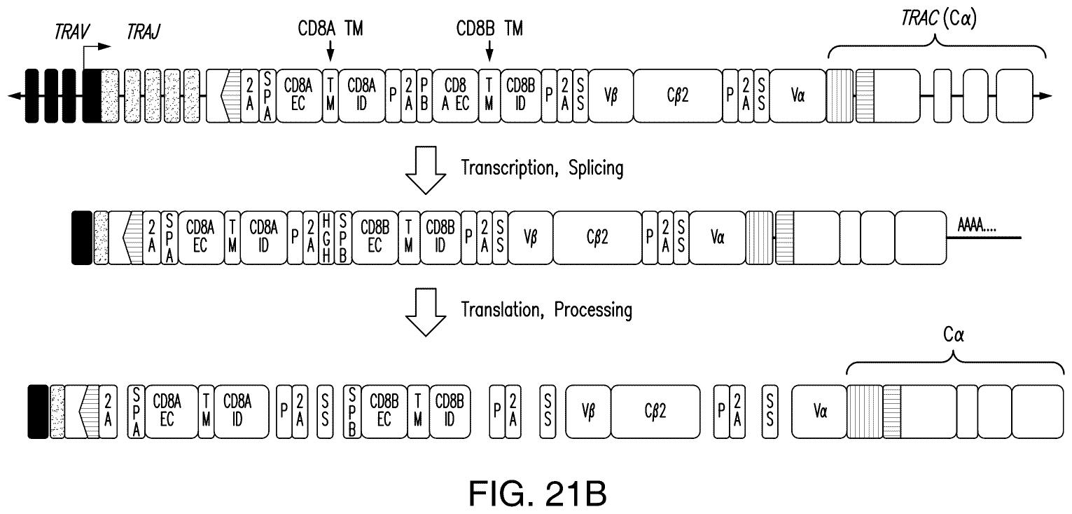

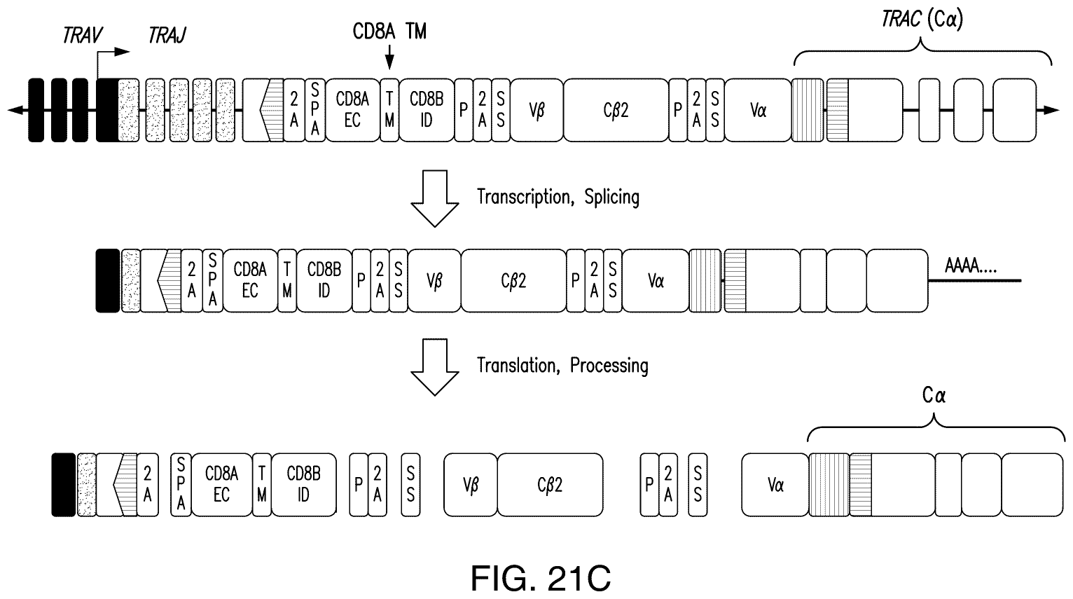

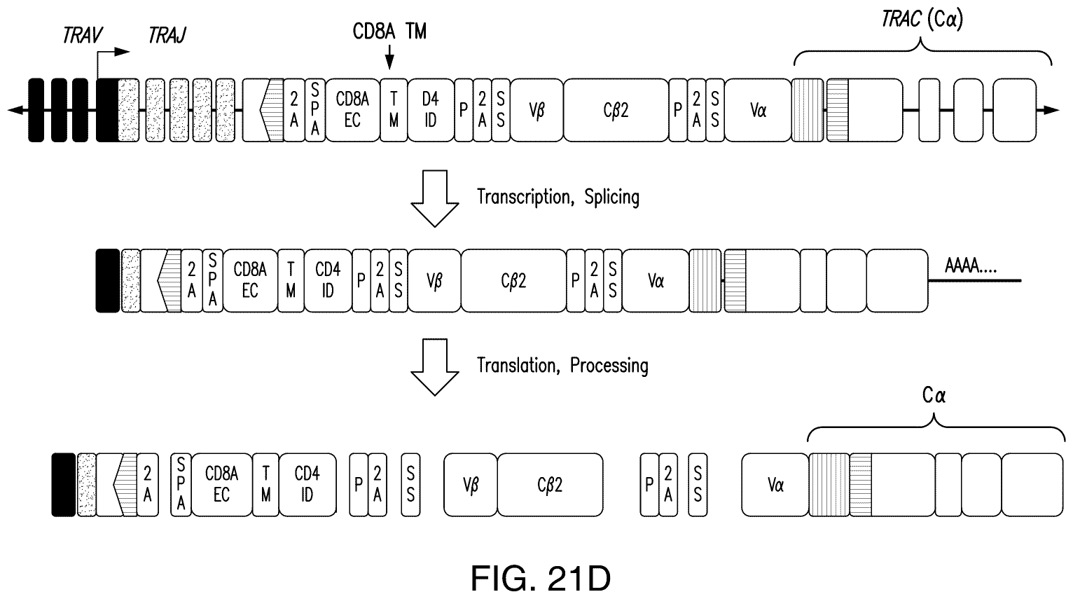

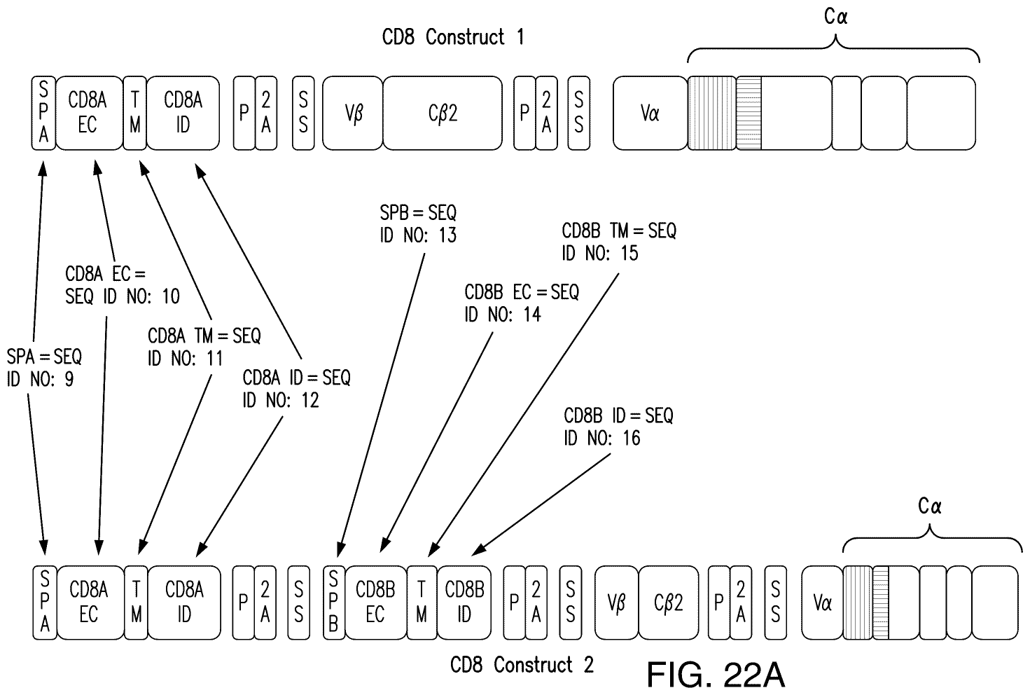

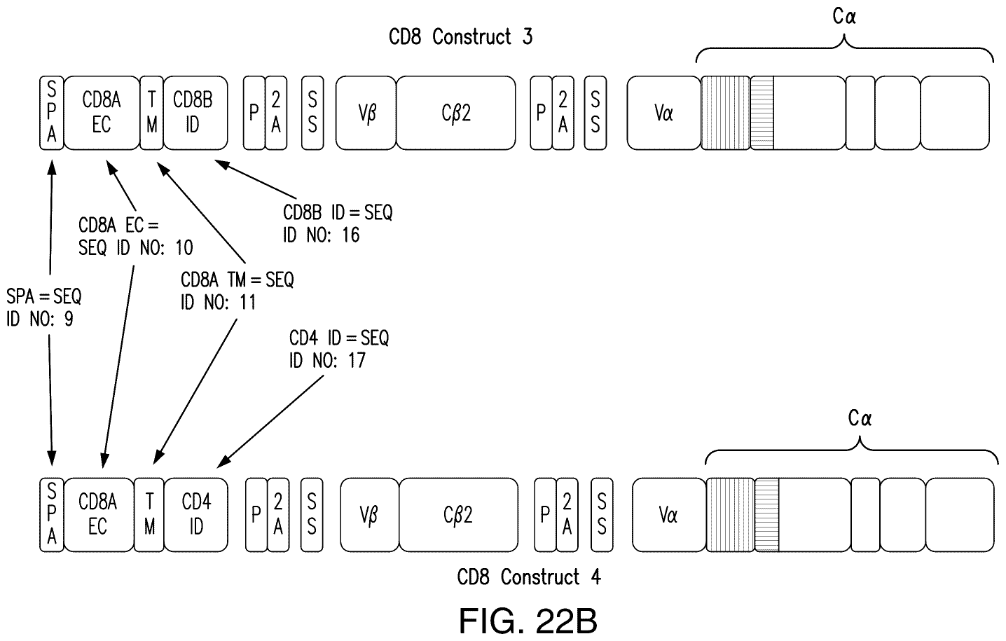

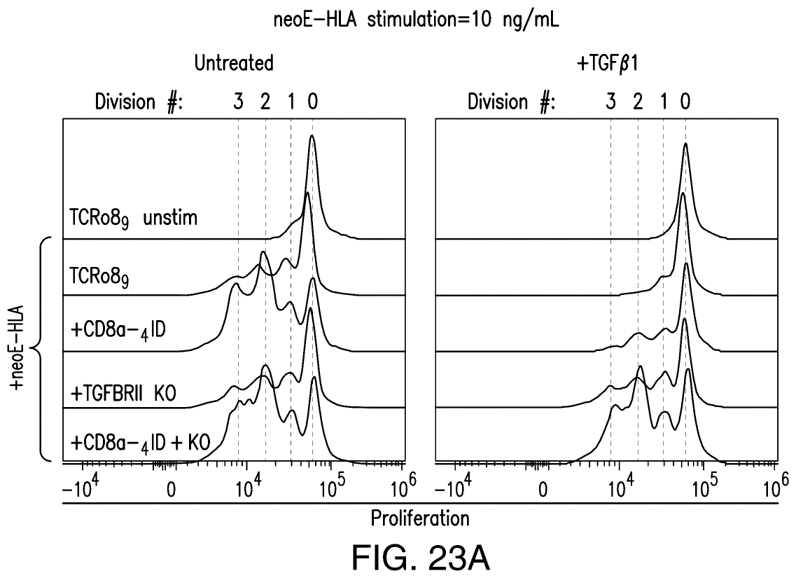

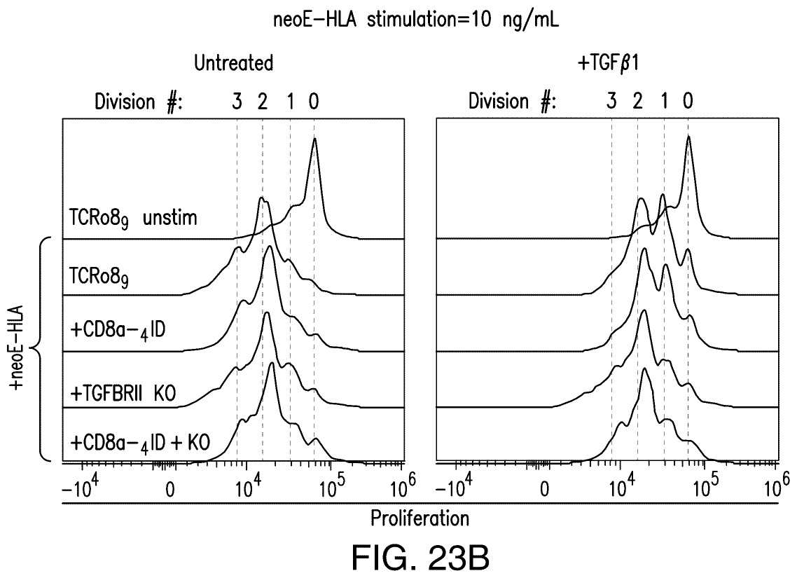

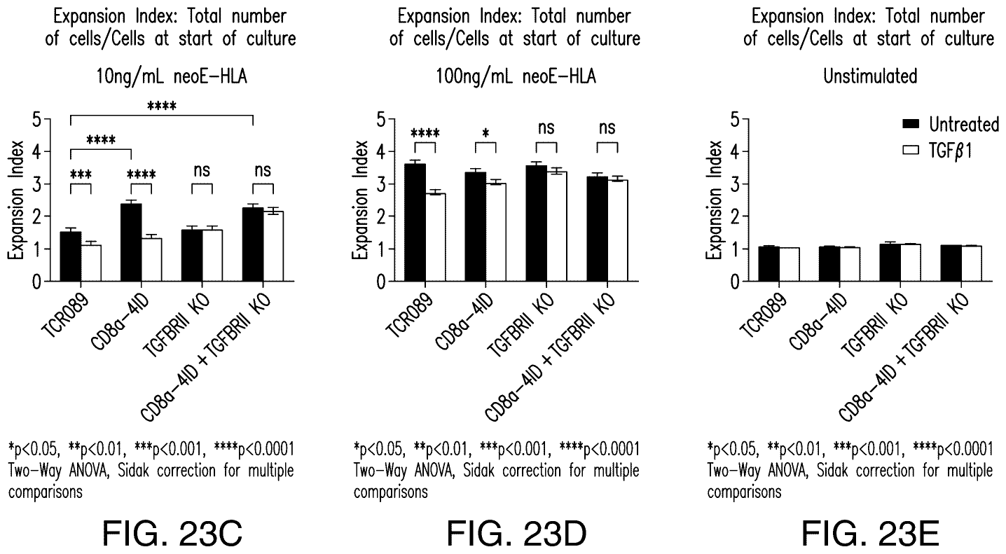

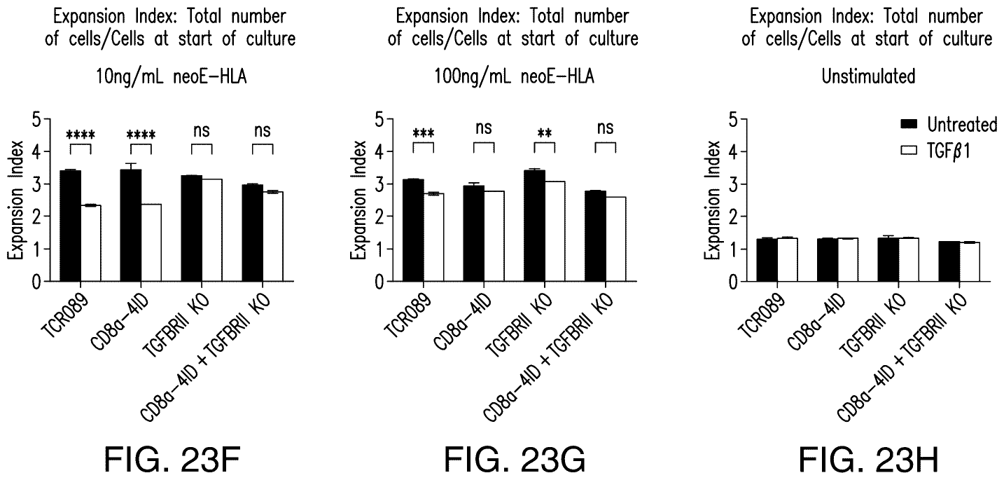

“CD8 Construct,” as used herein, means any one of a CD8 Construct 1, a CD8 Construct 2, a CD8 Construct 3, or a CD8 Construct 4. As used herein, “CD8 Construct 1” refers to a construct that comprises a NeoTCR and CD8α (CD8α extracellular domain, CD8α transmembrane domain, and CD8α intracellular domain). Non-limiting examples of CD8 Construct 1 is provided in FIGS. 20A, 21A, and 22A. As used herein, “CD8 Construct 2” refers to a construct that comprises a NeoTCR, CD8α (CD8α extracellular domain, CD8α transmembrane domain, and CD8α intracellular domain), and CD8β (CD8β extracellular domain, CD8β transmembrane domain, and CD8β intracellular domain). Non-limiting examples of CD8 Construct 2 is provided in FIGS. 20B, 21B, and 22A. As used herein, “CD8 Construct 3” refers to a construct that comprises a NeoTCR, CD8α extracellular domain, CD8α transmembrane domain, and CD8β intracellular domain. Non-limiting examples of CD8 Construct 3 is provided in FIGS. 22C, 21C, and 22B. As used herein, “CD8 Construct 4” and refer to a construct that comprises a NeoTCR, CD8α extracellular domain, CD8α transmembrane domain, and CD4 intracellular domain). Non-limiting examples of CD8 Construct 4 is provided in FIGS. 20D, 21D, and 22B.

As used herein, “CD8 Cells” refer to one or more cells precision engineered to express a NeoTCR and including an exogenous CD8 (e.g., expressed by one of CD8 constructs disclosed herein).

As used herein, “CD Product” refers to a product comprising CD8 Cells.

“CD8-TGFβRII Cells” as used herein means one or more cells precision engineered to 1) express one or more NeoTCRs, 2) express a CD8 Construct, and 3) engineered to completely or substantially knockout TGRβRII expression.

“CD8-TGFβRII Product” as used herein means a product comprising CD8-TGFβRII Cells.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, the NeoTCR Product of the invention is used to delay development of a proliferative disorder (e.g., cancer) or to slow the progression of such disease.

“Dextramer” as used herein means a multimerized neoepitope-HLA complex that specifically binds to its cognate NeoTCR.

The term “tumor antigen,” as used herein, refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

As used herein, the terms “neoantigen”, “neoepitope” or “neoE” refer to a newly formed antigenic determinant that arises, e.g., from a somatic mutation(s) and is recognized as “non-self.” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications. In certain embodiments, the neoantigen can be a private neoantigen. As used herein, the term “private neoantigen” refers to neoantigens that are exclusively expressed and present in a subject having certain cancer. For clarity, a private neoantigen is a neoantigen that cannot be used for another patient. In certain embodiments, the neoantigen can be a “public neoantigen.” The term “public neoantigen,” as used herein, refers to neoantigens that are shared by more than one subject.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product is not considered unacceptably toxic.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

As used herein, “2A” and “2A peptide” are used interchangeably herein and mean a class of 18-22 amino acid long, viral, self-cleaving peptides that are able to mediate cleavage of peptides during translation in eukaryotic cells. Four well-known members of the 2A peptide class are T2A, P2A, E2A, and F2A. The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus. The self-cleaving mechanism of the 2A peptides is a result of ribosome skipping the formation of a glycyl-prolyl peptide bond at the C-terminus of the 2A. Specifically, the 2A peptides have a C-terminal conserved sequence that is necessary for the creation of steric hindrance and ribosome skipping. The ribosome skipping can result in one of three options: 1) successful skipping and recommencement of translation resulting in two cleaved proteins (the upstream of the 2A protein which is attached to the complete 2A peptide except for the C-terminal proline and the downstream of the 2A protein which is attached to one proline at the N-terminal; 2) successful skipping but ribosome fall-off that results in discontinued translation and only the protein upstream of the 2A; or 3) unsuccessful skipping and continued translation (i.e., a fusion protein). In certain embodiments, the 2A peptide is a P2A peptide comprising the amino acid sequence set forth in SEQ ID NO: 18. In certain embodiments, the 2A peptide is a P2A peptide comprising the amino acid sequence set forth in SEQ ID NO: 19. In certain embodiments, the 2A peptide is a P2A peptide consisting of the amino acid sequence set forth in SEQ ID NO: 18. In certain embodiments, the 2A peptide is a P2A peptide consisting of the amino acid sequence set forth in SEQ ID NO: 19. SEQ ID NO: 18 and SEQ ID NO: 19 are provided below.

[SEQ ID NO: 18]
ATNFSLLKQAGDVEENPGP
[SEQ ID NO: 19]
GSGATNFSLLKQAGDVEENPGP

The term “endogenous,” as used herein, refers to a nucleic acid molecule or polypeptide that is normally expressed in a cell or tissue.

The term “exogenous,” as used herein, refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

“Young” or “Younger” or “Young T cell” as it relates to T cells means memory stem cells (T_{MSC}) and central memory cells (T_{CM}). These cells have T cell proliferation upon specific activation and are competent for multiple cell divisions. They also have the ability to engraft after re-infusion, to rapidly differentiate into effector T cells upon exposure to their cognate antigen and target and kill tumor cells, as well as to persist for ongoing cancer surveillance and control.

### 2. NeoTCR Products

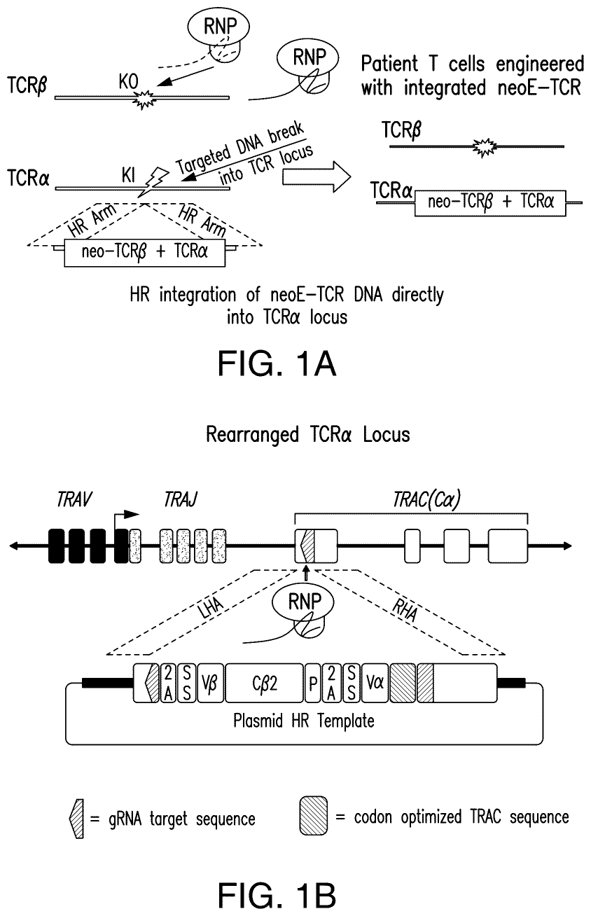

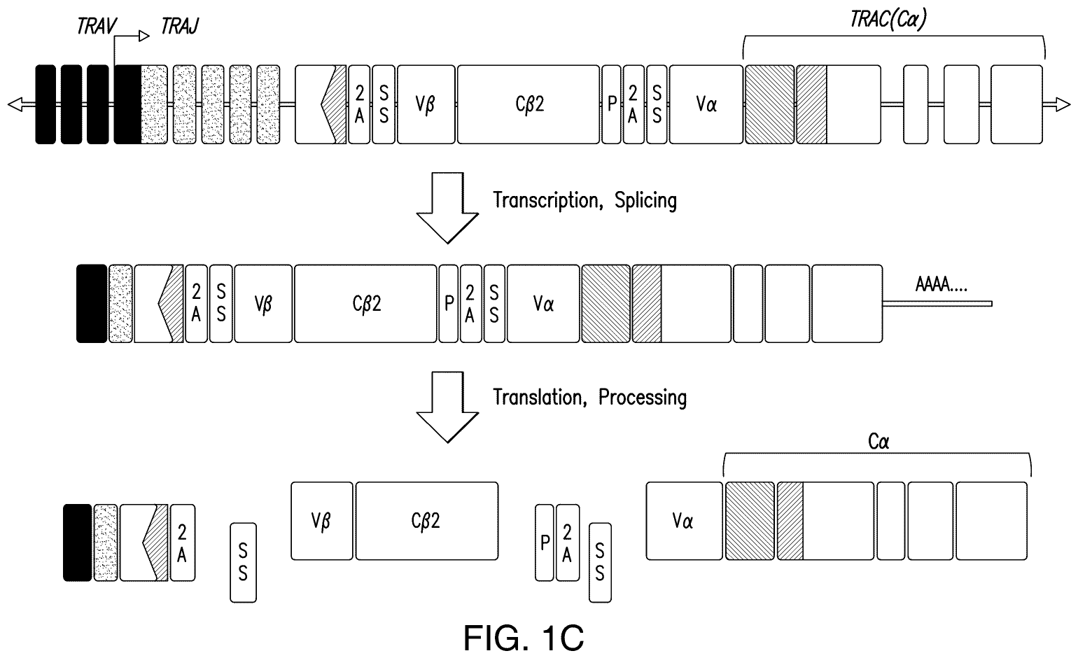

In certain embodiments, using the gene editing technology and NeoTCR isolation technology described in International Patent Applications Nos. PCT/US2020/17887 and PCT/US2019/025415, the contents of each of which are incorporated herein by reference in their entireties, NeoTCRs are cloned in autologous CD8+ and CD4+ T cells from the same patient with cancer by precision genome engineered (using a DNA-mediated (non-viral) method as described in FIGS. 1A-1C) to express the NeoTCR. In other words, the NeoTCRs that are tumor specific are identified in cancer patients, such NeoTCRs are then cloned, and then the cloned NeoTCRs are inserted into the cancer patient's T cells. NeoTCR expressing T cells are then expanded in a manner that preserves a “young” T cell phenotypes, resulting in a NeoTCR-P1 product (i.e., a NeoTCR Product) in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of NeoTCR Product, consisting significantly of ‘young’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Products manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating products with T cells from patients with cancer as starting material.

In certain embodiments, the NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Products or comparable cell products from healthy donors were assessed for cleavage of the candidate off-target sites by deep sequencing, supporting the published evidence that the selected nucleases are highly specific. Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables highly efficient generation of bespoke NeoTCR T cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

In certain embodiments, the NeoTCR Cells can include additional genomic modifications. In certain embodiments, the NeoTCR Cells can include a knockout of a TGFβR locus. In certain embodiments, the NeoTCR Cells can include an exogenous CD8 receptor. In certain embodiments, the NeoTCR Cells can include a knockout of a TGFβRII locus and an exogenous CD8 receptor.

### 2.1. TGFβRII Products

In certain embodiments, the present disclosure provides NeoTCR Cells including a knockout of a TGFβR locus.

Transforming growth factor beta receptors (TGFβR) are single pass serine/threonine kinase receptors that belong to TGFβ receptor family. They exist in several different isoforms that can be homo- or heterodimeric. The number of characterized ligands in the TGFβ superfamily far exceeds the number of known receptors, suggesting the promiscuity that exists between the ligand and receptor interactions. Three TGFβ superfamily receptors specific for TGFβ, the TGFβ receptors, can be distinguished by their structural and functional properties. TGFβRI and TGFβRII have similar ligand-binding affinities and can be distinguished from each other only by peptide mapping. Both TGFβRI and TGFβRII have a high affinity for TGFβ1 and low affinity for TGFβ2. TGFβRIII (β-glycan) has a high affinity for both homodimeric TGFβ1 and TGFβ2 and in addition the heterodimer TGF-β1.2. The TGFβ receptors also bind TGFβ3.

In certain embodiments, the NeoTCR Cells include a knockout of a TGFβRI locus. In certain embodiments, the NeoTCR Cells include a knockout of a TGFβRII locus. In certain embodiments, the NeoTCR Cells include a knockout of a TGFβRIII locus.

In certain embodiments, the NeoTCR Cells described herein can be TGFβRII Cells. In certain embodiments, the NeoTCR Products described herein can be TGFβRII Products.

In certain embodiments, the NeoTCR Products described above are further modified to knockout or substantially knock down TGFβRII expression from the cells of the product (i.e., a TGFβRII Product). Specifically, using the gene editing technology and NeoTCR isolation technology described in PCT/US2020/017887 and PCT/US2019/025415, which are incorporated herein in their entireties, NeoTCRs are cloned in autologous CD8+ and CD4+ T cells from the same patient with cancer by precision genome engineering (using a DNA-mediated (non-viral) method) to express the NeoTCR.

In certain embodiments, the TGFβRII Cells include a gene disruption of the TGFβRII locus that generates a knockout of the TGFβRII gene expression. In certain embodiments, the TGFβRII Cells include a gene disruption of the TGFβRII locus that generates a knockdown of the TGFβRII gene expression. In certain embodiments, the gene disruption of the TGFβRII locus can be obtained by any of the gene editing methods disclosed in Section 2.5 below. In certain non-limiting embodiments, the gene disruption is obtained by homologous recombination, non-homologous end joining, targeted nucleases (e.g., endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease).

In certain embodiments, the gene disruption of the TGFβRII locus can be a disruption of the coding region of the TGFβRII locus (e.g., exons). In certain embodiments, the gene disruption of the TGFβRII locus can be a disruption of the non-coding region of the TGFβRII locus (e.g., introns). Human TGFβRII locus includes seven exons: exon 1, exon 2, exon 3, exon 4, exon 5, exon 6, and exon 7. In certain embodiments, the gene disruption of the TGFβRII locus can be a disruption of the exon 1 of the TGFβRII locus. In certain embodiments, the gene disruption of the TGFβRII locus can be a disruption of the exon 2 of the TGFβRII locus.

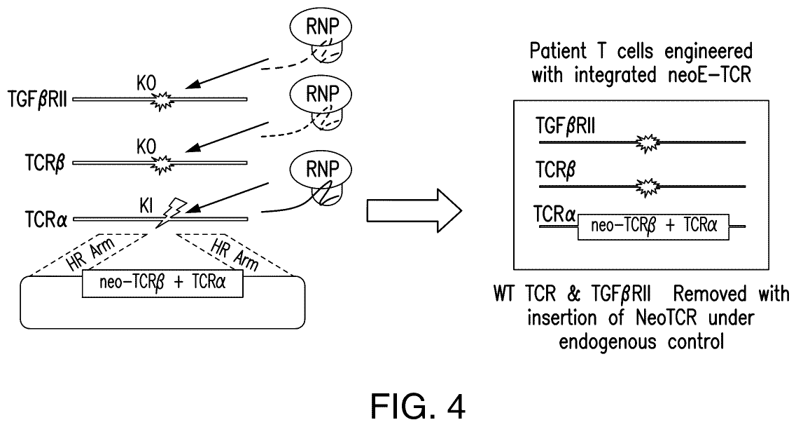

In certain embodiments, the TGFβRII gene is disrupted using a CRISPR/Cas nuclease system. In certain embodiments, the TGFβRII gene is disrupted using a gRNA to knockout expression of TGFβRII in the same reaction as the reaction used to knockout the TCRβ and insertion of the NeoTCRα and NeoTCRβ (see FIG. 4). In certain embodiments, the knockout of the TCRβ, insertion of the NeoTCRα and NeoTCRβ, and knockout of TGFβRII are performed concurrently in the same reaction.

In certain embodiments, the TGFβRII gene is disrupted using a gRNA to knockout expression of TGFβRII in a different reaction as the reaction used to knockout the TCRβ and insertion of the a and R chains of the NeoTCR (“NeoTCRα” and “NeoTCRβ,” respectively) (see FIG. 4). In certain embodiments, the knockout of the TCRβ and insertion of the NeoTCRα and NeoTCRβ are performed in separate and consecutive reactions from the knockout of TGFβRII. In certain embodiments, the knockout of the TCRβ and insertion of the NeoTCRα and NeoTCRβ are performed in a first reaction and the knockout of TGFβRII is performed in a second reaction. In certain embodiments, the knockout of TGFβRII is performed in a first reaction and the knockout of the TCRβ and insertion of the NeoTCRα and NeoTCRβ are performed in a second reaction.

In certain embodiments, the TGFβRII Products comprise cells that were engineered to express one or more NeoTCRs using viral methods (i.e., NeoTCR Viral Product) and a knockout of the TGFβRII gene. In certain embodiments, the cells of the NeoTCR Viral Product are further engineered to knockout the TGFβRII gene using non-viral methods. In certain embodiments, the cells of the NeoTCR Viral Product are further engineered to knockout the TGFβRII gene using viral methods.

In certain embodiments, the TGFβRII Product manufacturing process involves electroporation of 1) dual ribonucleoprotein species of CRISPR-Cas9 nucleases bound to guide RNA sequences, with each species targeting the genomic TCRα and the genomic TCRβ loci, and 2) ribonucleoprotein species of CRISPR-Cas9 nucleases bound to guide RNA sequences that target the TGFβRII gene. The specificity of targeting Cas9 nucleases to each genomic locus has been previously described in the literature as being highly specific. Comprehensive testing of the TGFβRII Product can be performed in vitro and in silico analyses to survey possible off-target genomic cleavage sites, using COSMID and GUIDE-seq, respectively. In certain embodiments, testing can be performed using COSMID-based in silico prediction of off-targets and GUIDE-seq-based in vitro prediction of off-targets, followed by testing of those putative off-targets by targeted deep sequencing.

In certain embodiments, the TGFβRII Products comprise TGFβRII Cells wherein the endogenous TGFβRII is knocked out. In certain embodiments, the TGFβRII Products comprise TGFβRII Cells wherein the endogenous TGFβRII is substantially knocked down.

In certain embodiments, the TGFβRII Products comprise TGFβRII Cells wherein the endogenous TGFβRII is knocked out using a CRISPR system. In certain embodiments, the TGFβRII Products comprise TGFβRII Cells wherein the endogenous TGFβRII is knocked out using the CRISPR/Cas9 system. In certain embodiments, the TGFβRII Products comprise TGFβRII Cells wherein the endogenous TGFβRII is substantially knocked down using a CRISPR system. In certain embodiments, the TGFβRII Products comprise TGFβRII Cells wherein the endogenous TGFRII is substantially knocked down using the CRISPR/Cas9 system. In certain embodiments, the TGFβRII Products comprise TGFβRII Cells wherein the endogenous TGFβRII is substantially knocked down using an shRNA.

In certain embodiments, the TGFβRII Products comprise TGFβRII Cells wherein the endogenous TGFβRII locus is knocked out using the CRISPR/Cas9 system with any of the gRNAs provided in Table 1. In certain embodiments, the TGFβRII Products comprise TGFβRII Cells wherein the endogenous TGFβRII locus is substantially knocked down using the CRISPR/Cas9 system with any of the gRNAs provided in Table 1.

TABLE 1

| gRNA Name | Gene | Exon | Sequence | SEQ ID NO: |
|---|---|---|---|---|
| Tgfbr2.sgRNA1 | TGFBR2 | 1 | CCGACUUCUG | SEQ ID NO: |
| | | | AACGUGCGGU | 1 |
| Tgfbr2.sgRNA2 | TGFBR2 | 1 | UCACCCGACU | SEQ ID NO: |
| | | | UCUGAACGUG | 2 |
| Tgfbr2.sgRNA3 | TGFBR2 | 2 | AUGAUAGUCA | SEQ ID NO: |
| | | | CUGACAACAA | 3 |

In certain embodiments, the TGFβRII Products comprise TGFβRII Cells that have a greater proliferative capacity than NeoTCR Cells expressing the same NeoTCR that does not comprise a TGFβRII knockout or substantial knockdown. In certain embodiments, the TGFβRII Cells are capable of proliferating at a higher rate as compared to NeoTCR Cells. In certain embodiments, TGFβRII Cells are capable of proliferating for longer periods of time as compared to NeoTCR Cells.

In certain embodiments, the TGFβRII Products comprise TGFβRII Cells that can proliferate in vitro for a period of time up to about two (2) weeks. In certain embodiments, the TGFβRII Products comprise TGFβRII Cells that proliferate in vitro for a period of time between about two (2) weeks and about one (1) month. In certain embodiments, the TGFβRII Products comprise TGFβRII Cells that can proliferate in vitro for a period of time between about one (1) month and about two (2) months. In certain embodiments, the TGFβRII Products comprise TGFβRII Cells that can proliferate in vitro for a period of time between about two (2) months and about three (3) months. In certain embodiments, the TGFβRII Products comprise TGFβRII Cells that can proliferate in vitro for a period of time between about three (3) months and about four (4) months. In certain embodiments, the TGFβRII Products comprise TGFβRII Cells that can proliferate in vitro for a period of time between about four (4) months and about five (5) months. In certain embodiments, the TGFβRII Products comprise TGFβRII Cells that proliferate in vitro for a period of time between about five (5) months and about six (6) months. In certain embodiments, the TGFβRII Products comprise TGFβRII Cells that can proliferate in vitro for a period of time greater than about six (6) months.

In certain embodiments, the TGFβRII Products comprise TGFβRII Cells that can proliferate ex vivo for a period of time up to about two (2) weeks. In certain embodiments, the TGFβRII Products comprise TGFβRII Cells that proliferate ex vivo for a period of time between about two (2) weeks and about one (1) month. In certain embodiments, the TGFβRII Products comprise TGFβRII Cells that can proliferate ex vivo for a period of time between about one (1) month and about two (2) months. In certain embodiments, the TGFβRII Products comprise TGFβRII Cells that can proliferate ex vivo for a period of time between about two (2) months and about three (3) months. In certain embodiments, the TGFβRII Products comprise TGFβRII Cells that can proliferate ex vivo for a period of time between about three (3) months and about four (4) months. In certain embodiments, the TGFβRII Products comprise TGFβRII Cells that can proliferate ex vivo for a period of time between about four (4) months and about five (5) months. In certain embodiments, the TGFβRII Products comprise TGFβRII Cells that proliferate ex vivo for a period of time between about five (5) months and about six (6) months. In certain embodiments, the TGFβRII Products comprise TGFβRII Cells that can proliferate ex vivo for a period of time greater than about six (6) months.

In certain embodiments, the TGFβRII Products comprise TGFβRII Cells that can proliferate in vivo for a period of time up to about two (2) weeks. In certain embodiments, the TGFβRII Products comprise TGFβRII Cells that can proliferate in vivo for a period of time between about two (2) weeks and about one (1) month. In certain embodiments, the TGFβRII Products comprise TGFβRII Cells that can proliferate in vivo for a period of time between about one (1) month and about two (2) months. In certain embodiments, the TGFβRII Products comprise TGFβRII Cells that can proliferate in vivo for a period of time between about two (2) months and about three (3) months. In certain embodiments, the TGFβRII Products comprise TGFβRII Cells that can proliferate in vivo for a period of time between about three (3) months and about four (4) months. In certain embodiments, the TGFβRII Products comprise TGFβRII Cells that can proliferate in vivo for a period of time between about four (4) months and about five (5) months. In certain embodiments, the TGFβRII Products comprise TGFβRII Cells that can proliferate in vivo for a period of time between about five (5) months and about six (6) months. In certain embodiments, the TGFβRII Products comprise TGFβRII Cells that can proliferate in vivo for a period of time for greater than about six (6) months.

In certain embodiments, the TGFβRII Products comprise TGFβRII Cells that can proliferate in vivo as long as there are tumor cells expressing the neoantigen cognate to the NeoTCR expressed by the TGFβRII Cells. In certain embodiments, the TGFβRII Products comprise TGFβRII Cells that can proliferate in vivo as long as there are cells expressing the neoantigen cognate to the NeoTCR expressed by the TGFβRII Cells. In certain embodiments, the TGFβRII Products comprise TGFβRII Cells that can only proliferate in vivo in the presence of a cell that expresses a neoantigen cognate to the NeoTCR expressed by the TGFβRII Cells. In certain embodiments, in vivo means in a patient following infusion of a TGFβRII Product.

In certain embodiments, the TGFβRII Products comprise TGFβRII Cells that can persist longer in patient's post-infusion than a NeoTCR Product expressing the same NeoTCR and that does not comprise a TGFβRII knockout or substantial knockdown thereof.

In certain embodiments, the TGFβRII Products comprise TGFβRII Cells that can persist in vitro for a period of time up to about two (2) weeks. In certain embodiments, the TGFβRII Products comprise TGFβRII Cells that can persist in vitro for a period of time between about two (2) weeks and about one (1) month. In certain embodiments, the TGFβRII Products comprise TGFβRII Cells that can persist in vitro for a period of time between about one (1) month and about two (2) months. In certain embodiments, the TGFβRII Products comprise TGFβRII Cells that can persist in vitro for a period of time between about two (2) months and about three (3) months. In certain embodiments, the TGFβRII Products comprise TGFβRII Cells that can persist in vitro for a period of time between about three (3) months and about four (4) months. In certain embodiments, the TGFβRII Products comprise TGFβRII Cells that can persist in vitro for a period of time between about four (4) months and about five (5) months. In certain embodiments, the TGFβRII Products comprise TGFβRII Cells that can persist in vitro for a period of time between about five (5) months and about six (6) months. In certain embodiments, the TGFβRII Products comprise TGFβRII Cells that can persist in vitro for a period of time greater than about six (6) months.

In certain embodiments, the TGFβRII Products comprise TGFβRII Cells that can persist ex vivo for a period of time up to about two (2) weeks. In certain embodiments, the TGFβRII Products comprise TGFβRII Cells that can persist ex vivo for a period of time between about two (2) weeks and about one (1) month. In certain embodiments, the TGFβRII Products comprise TGFβRII Cells that can persist ex vivo for a period of time between about one (1) month and about two (2) months. In certain embodiments, the TGFβRII Products comprise TGFβRII Cells that can persist ex vivo for a period of time between about two (2) months and about three (3) months. In certain embodiments, the TGFβRII Products comprise TGFβRII Cells that can persist ex vivo for a period of time between about three (3) months and about four (4) months. In certain embodiments, the TGFβRII Products comprise TGFβRII Cells that can persist ex vivo for a period of time between about four (4) months and about five (5) months. In certain embodiments, the TGFβRII Products comprise TGFβRII Cells that can persist ex vivo for a period of time between about five (5) months and about six (6) months. In certain embodiments, the TGFβRII Products comprise TGFβRII Cells that can persist ex vivo for a period of time greater than about six (6) months.

In certain embodiments, the TGFβRII Products comprise TGFβRII Cells that can persist in vivo for a period of time up to about two (2) weeks. In certain embodiments, the TGFβRII Products comprise TGFβRII Cells that can persist in vivo for a period of time between about two (2) weeks and about one (1) month. In certain embodiments, the TGFβRII Products comprise TGFβRII Cells that can persist in vivo for a period of time between about one (1) month and about two (2) months. In certain embodiments, the TGFβRII Products comprise TGFβRII Cells that can persist in vivo for a period of time between about two (2) months and about three (3) months. In certain embodiments, the TGFβRII Products comprise TGFβRII Cells that can persist in vivo for a period of time between about three (3) months and about four (4) months. In certain embodiments, the TGFβRII Products comprise TGFβRII Cells that can persist in vivo for a period of time between about four (4) months and about five (5) months. In certain embodiments, the TGFβRII Products comprise TGFβRII Cells that can persist in vivo for a period of time between about five (5) months and about six (6) months. In certain embodiments, the TGFβRII Products comprise TGFβRII Cells that can persist in vivo for a period of time greater than about six (6) months.

In certain embodiments, the TGFβRII Products comprise TGFβRII Cells that can persist in vivo as long as there are tumor cells expressing the neoantigen cognate to the NeoTCR expressed by the TGFβRII Cells. In certain embodiments, the TGFβRII Products comprise TGFβRII Cells that can persist in vivo as long as there are cells expressing the neoantigen cognate to the NeoTCR expressed by the TGFβRII Cells. In certain embodiments, the TGFβRII Products comprise TGFβRII Cells that can only persist in vivo in the presence of a cell that expresses a neoantigen cognate to the NeoTCR expressed by the TGFβRII Cells. In certain embodiments, in vivo means in a patient following infusion of a TGFβRII Product.

In certain embodiments, TGFβRII Cells are expanded in vitro or ex vivo in a manner that preserves a “young” T cell phenotypes, resulting in a TGFβRII Product in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of TGFβRII Product including significant amounts of ‘young’ T cell, has the potential to benefit patients with cancer, through improved engraftment potential, prolonged persistence post-infusion, and rapid differentiation into effector T cells to eradicate tumor cells throughout the body.

In certain embodiments, the TGFβRII cells of the TGFβRII Products predominantly comprise memory stem cells (T_{msc}) and/or central memory cells (Tₑₘ). In certain embodiments, at least about 25% of the TGFβRII cells of the TGFβRII Products comprise memory stem cells (T_{msc}) and/or central memory cells (Tₑₘ). In certain embodiments, at least about 30% of the TGFβRII cells of the TGFβRII Products comprise memory stem cells (T_{msc}) and/or central memory cells (Tₑₘ). In certain embodiments, at least about 35% of the TGFβRII cells of the TGFβRII Products comprise memory stem cells (T_{msc}) and/or central memory cells (T_{cm}). In certain embodiments, at least about 40% of the TGFβRII cells of the TGFβRII Products comprise memory stem cells (T_{msc}) and/or central memory cells (T_{cm}). In certain embodiments, at least about 45% of the TGFβRII cells of the TGFβRII Products comprise memory stem cells (T_{msc}) and/or central memory cells (Tₑₘ). In certain embodiments, at least about 50% of the TGFβRII cells of the TGFβRII Products comprise memory stem cells (T_{msc}) and/or central memory cells (Tₑₘ). In certain embodiments, at least about 55% of the TGFβRII cells of the TGFβRII Products comprise memory stem cells (T_{msc}) and/or central memory cells (Tₑₘ). In certain embodiments, at least about 60% of the TGFβRII cells of the TGFβRII Products comprise memory stem cells (T_{msc}) and/or central memory cells (Tₑₘ). In certain embodiments, at least about 65% of the TGFβRII cells of the TGFβRII Products comprise memory stem cells (T_{msc}) and/or central memory cells (Tₑₘ). In certain embodiments, at least about 70% of the TGFβRII cells of the TGFβRII Products comprise memory stem cells (T_{msc}) and/or central memory cells (Tc T_{cm} m). In certain embodiments, at least about 75% of the TGFβRII cells of the TGFβRII Products comprise memory stem cells (T_{msc}) and/or central memory cells (T_{cm}). In certain embodiments, greater than about 75% of the TGFβRII cells of the TGFβRII Products comprise memory stem cells (T_{msc}) and/or central memory cells (Tₑₘ). In certain embodiments, T_{msc} can be CD45RA⁺CD62L⁺, CD28⁺CD95⁺, and CCR7⁺CD27⁺. In certain embodiments, T_{cm} can be CD45RO⁺CD62L⁺, CD28⁺CD95⁺, and CCR7⁺CD27⁺CD127⁺. Both Tₘₛ and T_{cm} are characterized as having weak effector T cell function, robust proliferation, robust engraftment, and long telomeres.

2.2. dnTGFβRII Products

In certain embodiments, the present disclosure provides NeoTCR Cells including a dominant negative TGFβR (dnTGFβR). In certain embodiments, the NeoTCR Cells include a dominant negative TGFβRI (dnTGFβRI). In certain embodiments, the NeoTCR Cells include a dominant negative TGFβRII (dnTGFβRII). In certain embodiments, the NeoTCR Cells include a dominant negative TGFβRIII (dnTGFβRIII).

In certain embodiments, the NeoTCR Cells described herein can be dnTGFβRII Cells. In certain embodiments, the NeoTCR Products described herein can be dnTGFβRII Products.

In certain embodiments, the dnTGFβRII Cells described herein are made by inserting a dnTGFβRII Construct in a cell using viral or non-viral methods that are designed to allow for the dual expression of a NeoTCR and a dnTGFβRII. In certain embodiments, the method of gene insertion is non-viral transfection. In certain embodiments, the non-viral transfection methods used are those described herein. In certain embodiments, the cell is a primary human cell. In certain embodiments, the primary human cell is a T cell.

In certain embodiments, one or more of the following key elements are included in the dnTGFβRII Constructs: an element to promote translation of transcripts (e.g., enhancer), a poly-adenylation (poly-A) sequence, a promoter, a pause element, a woodchuck hepatitis virus post-transcriptional regulatory element (WPRE), a scaffold/matrix attachment region, and an insulator.

In certain embodiments, the dnTGFβRII Constructs are modular, as each element should be thought of as a broad and general category. For instance, any poly-A signal sequence known to one of skill in the art could be used in the modular construct. For instance, any element to promote translation of transcripts applicable to the intended cell known to one of skill in the art could be used in the modular construct. For instance, any applicable promoter for the intended cell known to one of skill in the art could be used in the modular construct. For instance, any applicable insulator for the intended cell known to one of skill in the art could be used in the modular construct.

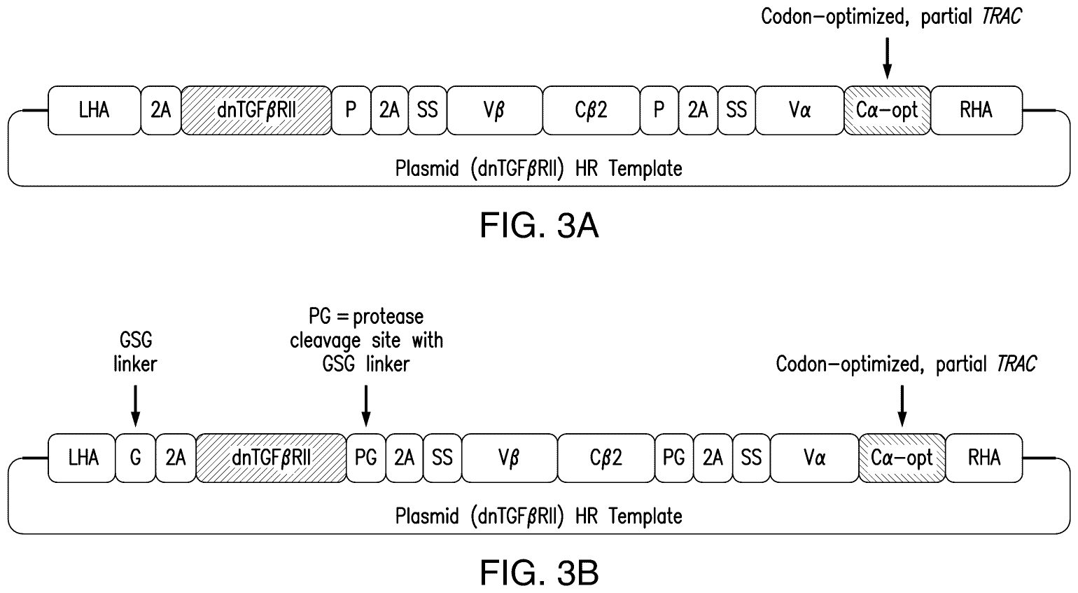

In certain embodiments, the dnTGFβRII Construct design is described in FIGS. 3A-3B and 13A-19. In certain non-limiting embodiments, the dnTGFβRII Construct design has the structure of a construct as depicted in FIGS. 3A and 3B. In certain embodiments, the dnTGFβRII Construct design is designated as “Format 1,” “Format 2,” “Format 3,” or “Format 4.”

In certain embodiments, the dnTGFβRII Construct includes one poly-A signal sequence. In certain non-limiting embodiments, the poly-A signal sequence can be a simian virus 40 (SV40) poly-A signal sequence, an SV40 poly-A signal sequence, a human growth hormone (hGH) poly-A signal sequence, a bovine growth hormone (BGH) poly-A signal sequence, or a rabbit beta-globin (rbGlob) poly-A signal sequence. In certain embodiments, the dnTGFβRII Construct includes two poly-A signal sequences. In certain embodiments, the two poly-A signal sequences are the same. In certain embodiments, the two poly-A signal sequences are different.

In certain embodiments, the poly-A signal sequence used in the dnTGFβRII Construct is the BGH poly-A signal sequence. In certain embodiments, the BGH poly-A signal sequence comprises a nucleotide sequence that is at least about 80%, about 85%, about 90%, about 95%, about 96%, about 97%, about 98%, or about 99% identical to the nucleotide sequence set forth in SEQ ID NO: 20. In certain embodiments, the BGH poly-A signal sequence comprises the nucleotide sequence set forth in SEQ ID NO: 20. In certain embodiments, the BGH poly-A signal sequence consists of the nucleotide sequence set forth in SEQ ID NO: 20. SEQ ID NO: 20 is provided below.

[SEQ ID NO: 20]
TGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAG
GAAATTGCATCGCATTGTCTGAGTAGGTGTCATTCTATTCTGGGGGGTGG
GGTGGGGCAGGACAGCAAGGGGGAGGATTGGGAAGACAATAGCAGGCATG
CTGGGGATGCGGTGGGCTCTATGGC

In certain embodiments, the poly-A signal sequence used in the dnTGFβRII Construct is the SV40 poly-A signal sequence. In certain embodiments, the SV40 poly-A signal sequence comprises a nucleotide sequence that is at least about 80%, about 85%, about 90%, about 95%, about 96%, about 97%, about 98%, or about 99% identical to the nucleotide sequence set forth in SEQ ID NO: 21. In certain embodiments, the SV40 poly-A signal sequence comprises the nucleotide sequence set forth in SEQ ID NO: 21. In certain embodiments, the SV40 poly-A signal sequence consists of the nucleotide sequence set forth in SEQ ID NO: 21. SEQ ID NO: 21 is provided below.

[SEQ ID NO: 21]
GCTTTATTTGTGAAATTTGTGATGCTATTGCTTTATTTGTAACCATTATA
AGCTGCAATAAACAAGTTAACAACAACAATTGCATTCATTTTATGTTTCA
GGTTCAGGGGGAGATGTGGGAGGTTTTTTAAAGC

In certain embodiments, the SV40 poly-A signal sequence further comprises an SV40 upstream element. In certain embodiments, the SV40 upstream element comprises a nucleotide sequence that is at least about 80%, about 85%, about 90%, about 95%, about 96%, about 97%, about 98%, or about 99% identical to the nucleotide sequence set forth in SEQ ID NO: 22. In certain embodiments, the SV40 upstream element comprises the nucleotide sequence set forth in SEQ ID NO: 22. In certain embodiments, the SV40 upstream element consists of the nucleotide sequence set forth in SEQ ID NO: 22. SEQ ID NO: 22 is provided below.

[SEQ ID NO: 22]
TTTATTTGTGAAATTTGTGATGCTATTGCTTTATTTGTAACCAT

In certain embodiments, the poly-A signal sequence used in the dnTGFβRII Construct is a 6T sequence. In certain embodiments, the 6T sequence comprises a nucleotide sequence that is at least about 80%, about 85%, about 90%, about 95%, about 96%, about 97%, about 98%, or about 99% identical to the nucleotide sequence set forth in SEQ ID NO: 23. In certain embodiments, the 6T poly-A signal sequence comprises the nucleotide sequence set forth in SEQ ID NO: 23. In certain embodiments, the 6T poly-A signal sequence consists of the nucleotide sequence set forth in SEQ ID NO: 23. SEQ ID NO: 23 is provided below.

[SEQ ID NO: 23]
TTTTTT

In certain embodiments, the poly-A signal sequence used in the dnTGFβRII Construct is the BGH poly-A signal sequence. In certain embodiments, the poly-A signal sequence used in the dnTGFβRII Construct is the SV40 poly-A signal sequence.

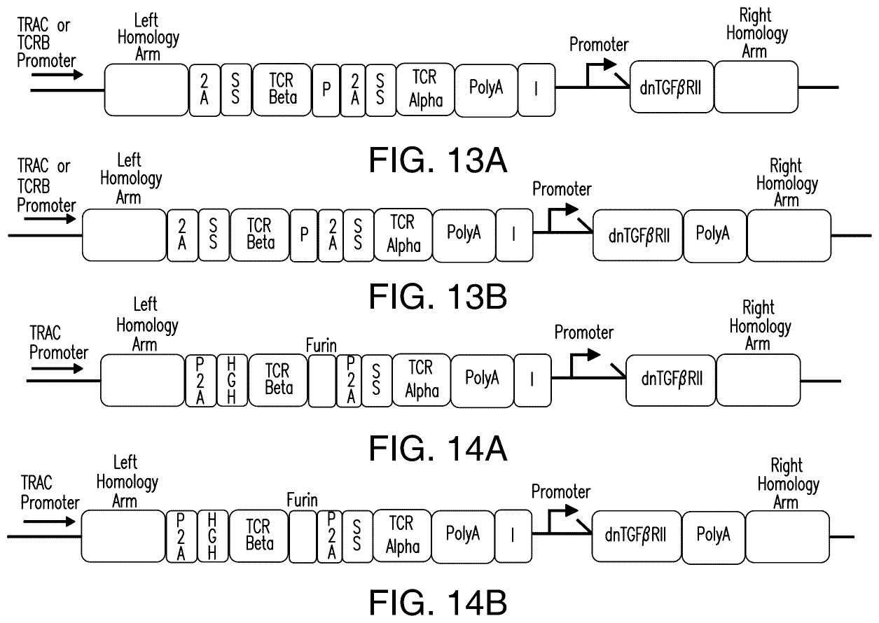

In certain embodiments, the poly-A signal sequence used in the dnTGFβRII Constructs shown in FIGS. 13A and 14A is the BGH poly-A signal sequence. In certain embodiments, the poly-A signal sequence used in the dnTGFβRII Constructs, and shown in FIGS. 13A and 14A, is the SV40 poly-A signal sequence.

In certain embodiments, the first poly-A signal sequence used in the dnTGFβRII Constructs shown in FIGS. 13B and 14B is the BGH poly-A signal sequence and the first poly-A signal sequence used in the dnTGFβRII Constructs shown in FIGS. 13B and 14B is the SV40 poly-A signal sequence. In certain embodiments, the first poly-A signal sequence used in the dnTGFβRII Constructs shown in FIGS. 13B and 14B is the SV40 poly-A signal sequence and the first poly-A signal sequence used in the dnTGFβRII Constructs shown in FIGS. 13B and 14B is the BGH poly-A signal sequence.

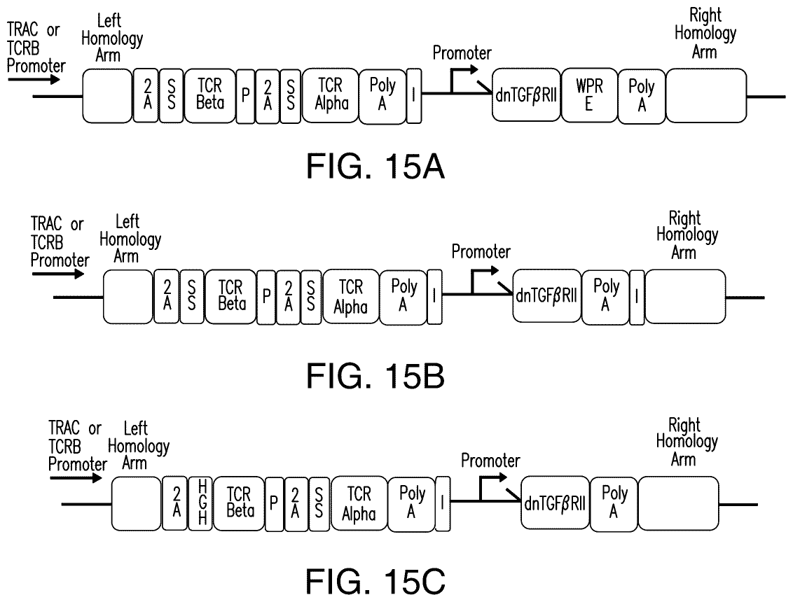

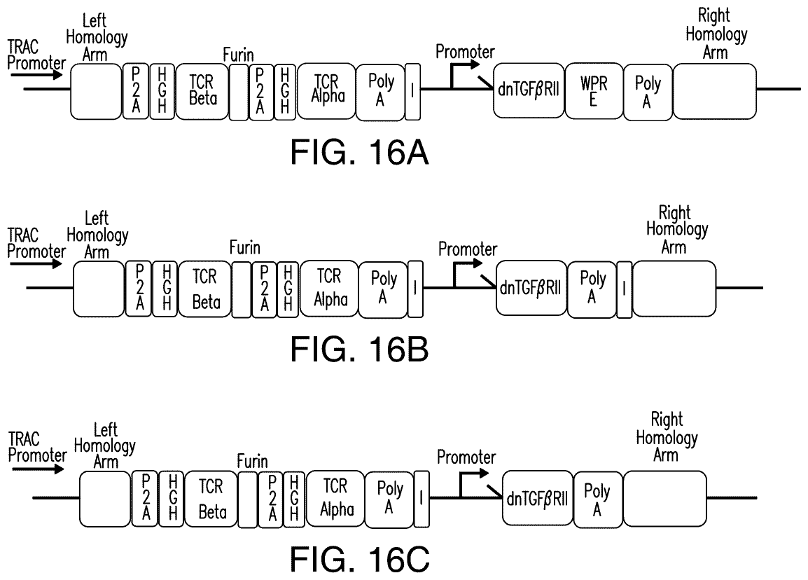

In certain embodiments, the first poly-A signal sequence used in the dnTGFβRII Constructs shown in FIGS. 15A-15C and 16A-16C is the BGH poly-A signal sequence and the first poly-A signal sequence used in the dnTGFβRII Constructs shown in FIGS. 15A-15C and 16A-16C is the SV40 poly-A signal sequence. In certain embodiments, the first poly-A signal sequence used in the dnTGFβRII Constructs shown in FIGS. 15A-15C and 16A-16C is the SV40 poly-A signal sequence and the first poly-A signal sequence used in the dnTGFβRII Constructs shown in FIGS. 15A-15C and 16A-16C is the BGH poly-A signal sequence.

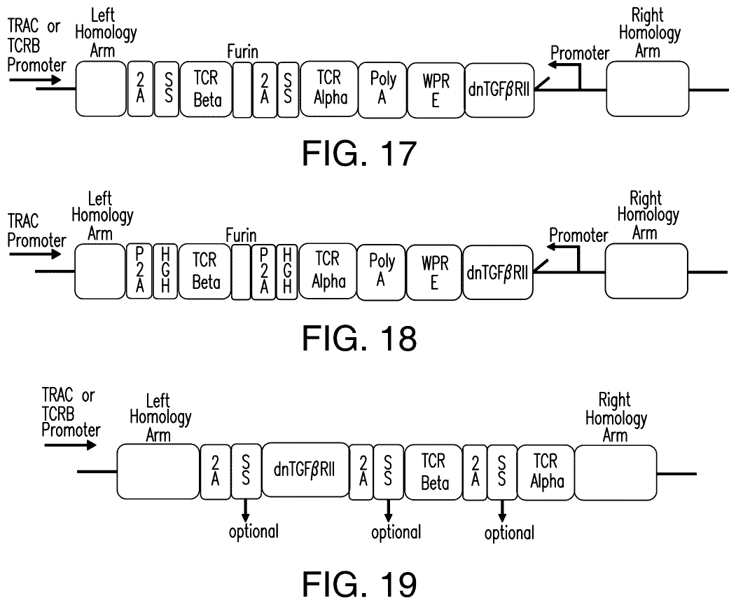

In certain embodiments, the poly-A signal sequence used in the dnTGFβRII Constructs shown in FIGS. 17 and 18 is the BGH poly-A signal sequence. In certain embodiments, the poly-A signal sequence used in the dnTGFβRII Constructs shown in FIGS. 17 and 18 is the SV40 poly-A signal sequence.

In certain embodiments, the dnTGFβRII Constructs disclosed herein include a transcriptional insulator or insulator. Insulators are a type of DNA sequence that helps divide the genome into distinct “genetic neighborhoods.” Insulators can help prevent regulatory elements designed to affect the expression of one gene from also affecting the expression of another nearby gene.

In certain embodiments, insulators can reduce promoter interference. Promoter interference is a molecular event characterized by the perturbation of one transcription unit by another. For example, without any limitation, promoter interference can occur between the transcription units of the genes of the dnTGFβRII Constructs (e.g., the sequence encoding a NeoTCR and the dnTGFβRII). Additional information on promoter interference can be found in Eszterhas et al., Molecular and Cellular Biology 22.2 (2002): 469-479.

In certain embodiments, promoter interference occurs when the expression of the dnTGFβRII reduces the NeoTCR expression. Accordingly, in certain embodiments, an insulator can be inserted into the dnTGFβRII Constructs to prevent reduction in NeoTCR expression.

In certain embodiments, insulators reduce promoter leakiness. Accordingly, in certain embodiments, insulators can be used to reduce transgene silencing.

In certain embodiments, an insulator is added to the construct between the NeoTCR gene and the dnTGFβRII. In certain embodiments, an insulator is added to the construct between the NeoTCR gene and the promoter region.

In certain embodiments, the insulators used in the dnTGFβRII Constructs are HS4 or IS2. Additional examples of insulators encompassed by the present disclosure include, without any limitation, CTCF insulator, Cohesin insulator, TFIIIC insulator, Condensin insulator, p68 insulator, PARP1 insulator, Bptf insulator, TGF-β insulator, and Kaiso insulator. Further information and examples of insulators can be found in Liu et al., Nature biotechnology 33.2 (2015): 198-203.

In certain embodiments, the insulator is an HS4 insulator. In certain embodiments, the HS4 insulator comprises a nucleotide sequence that is at least about 80%, about 85%, about 90%, about 95%, about 96%, about 97%, about 98%, or about 99% identical to the nucleotide sequence set forth in SEQ ID NO: 24. In certain embodiments, the HS4 insulator comprises the nucleotide sequence set forth in SEQ ID NO: 24. In certain embodiments, the HS4 insulator consists of the nucleotide sequence set forth in SEQ ID NO: 24. SEQ ID NO: 24 is provided below:

[SEQ ID NO: 24]
GAGCTCACGGGGACAGCCCCCCCCCAAAGCCCCCAGGGATGTAATTACGT
CCCTCCCCCGCTAGGGGGCAGCAGCGAGCCGCCCGGGGCTCCGCTCCGGT
CCGGCGCTCCCCCCGCATCCCCGAGCCGGCAGCGTGCGGGGACAGCCCGG
GCACGGGGAAGGTGGCACGGGATCGCTTTCCTCTGAACGCTTCTCGCTGC
TCTTTGAGCCTGCAGACACCTGGGGGGATACGGGGAAAAAGCTT

In certain embodiments, the insulator is an IS2 insulator. In certain embodiments, the IS2 insulator comprises a nucleotide sequence that is at least about 80%, about 85%, about 90%, about 95%, about 96%, about 97%, about 98%, or about 99% identical to the nucleotide sequence set forth in SEQ ID NO: 25. In certain embodiments, the IS2 insulator comprises the nucleotide sequence set forth in SEQ ID NO: 25. In certain embodiments, the IS2 insulator consists of the nucleotide sequence set forth in SEQ ID NO: 25. SEQ ID NO: 25 is provided below:

[SEQ ID NO: 25]
CGGGGACAGCCCCCCCCCAAAGCCCCCAGGGATGTAATTACGTCCCTCCC
CCGCTAGGGGGCAGCAGCGAGCCGCCCGGGGCTCCGCTCCGGTCCGGCGC
TCCCCCCGCATCCCCGAGCCGGCAGCGTGCGGGGACAGCCCGGGCACGGG
GAAGGTGGCACGGGATCGCTTTCCTCTGAACGCTTCTCGCTGCTCTTTGA
GCCTGCAGACACCTGGGGGGATACGGGGAAAATGTGTCTGAGCCTGCATG
TTTGATGGTGTCTGGATGCAAGCAGAAGGGGTGGAAGAGCTTGCCTGGAG
AGATACAGCTGGGTCAGTAGGACTGGGACAGGCAGCTGGAGAATTGCCAT
GTAGATGTTCATACAATCGTCAAATCATGAAGGCTGGAAAAGCCCTCCAA
GATCCCCAAGACCAACCCCAACCCACCCACCGTGCCCACTGGCCATGTCC
CTCAGTGCCACATCCCCACAGTTCTTCATCACCTCCAGGGACGGTGACCC
CCCCACCTCCGTGGGCAGCTGTGCCACTGCAGCACCGCTCTTTGGAGAAG
GTAAATCTTGCTAAATCCAGCCCGACCCTCCCCTGGCACAACGTAAGGCC
ATTATCTCTCATCCAACTCCAGGACGGAGTCAGTGAGAATATTTAAATAA
ACTTATAAATTGTGAGAGAAATTAATGAATGTCTAAGTTAATGCAGAAAC
GGAGGCTCCTCATTTATTTTTGAACTTAAAGACTTAATATTGTGAAGGTA
TACTTTCTTTAATAATAAGCCTGCGCCCAATATGTTCACCCCAAAAAAGC
TGTTTGTTAACTTGTCAACCTCATTTAAAATATATAAGAAACAGCCCAAA
GACAATAACAAAAGAATAATAAAAAAGAATGAAATATGTAATTCTTTCAG
AGTAAAAATCACACCCATGACCTGGCCACTGAGGGCTTGATCAATTCACT
TTGAATTTGGCATTAAATACCATTAAGGTATATTAACTGATTTTAAAATA
AGATATATTC

In certain embodiments, the dnTGFβRII Construct includes one insulator. In certain embodiments, the dnTGFβRII Construct includes two insulators. In certain embodiments, the two insulators are the same. In certain embodiments, the two insulators are different.

In certain embodiments, the insulator used in Format 1 constructs is HS4 (FIGS. 13A, 13B, 14A, and 14B). In certain embodiments, the insulator used in Format 1 is IS2 (FIGS. 13A, 13B, 14A, and 14B).

In certain embodiments, the insulator used in Format 2 constructs that only use 1 insulator is HS4 (FIGS. 15A, 15C, 16A, and 16C). In certain embodiments, the insulator used in Format 2 constructs that only use 1 insulator is IS2 (FIGS. 15A, 15C, 16A, and 16C). In certain embodiments where Format 2 constructs use two insulators (FIGS. 15B and 16B), either both insulators are HS4, both insulators are IS2, the first insulator is HS4 and the second insulator is IS2, or the first insulator is IS2 and the second insulator is HS4.

In certain embodiments, sequence elements that promote translation of transcripts are introduced into the dnTGFβRII Constructs. In certain embodiments, sequence elements that promote normal processing of intron-free transcripts are introduced into the dnTGFβRII Constructs. In certain embodiments, the sequence elements that promote normal processing of intron-free transcripts that are introduced into the dnTGFβRII Constructs are woodchuck hepatitis virus post-transcriptional regulatory elements (WPREs). In certain embodiments, WPREs are often used to promote normal processing of intron-free transcripts, resulting in normal levels of protein expression.

In certain embodiments, the WPRE is a WPRE3 element. In certain embodiments, the WPRE3 element comprises a nucleotide sequence that is at least about 80%, about 85%, about 90%, about 95%, about 96%, about 97%, about 98%, or about 99% identical to the nucleotide sequence set forth in SEQ ID NO: 26. In certain embodiments, the WPRE3 element comprises the nucleotide sequence set forth in SEQ ID NO: 26. In certain embodiments, the WPRE3 element consists of the nucleotide sequence set forth in SEQ ID NO: 26. SEQ ID NO: 26 is provided below:

[SEQ ID NO: 26]
GATAATCAACCTCTGGATTACAAAATTTGTGAAAGATTGACTGGTATTCT
TAACTATGTTGCTCCTTTTACGCTATGTGGATACGCTGCTTTAATGCCTT
TGTATCATGCTATTGCTTCCCGTATGGCTTTCATTTTCTCCTCCTTGTAT
AAATCCTGGTTAGTTCTTGCCACGGCGGAACTCATCGCCGCCTGCCTTGC
CCGCTGCTGGACAGGGGCTCGGCTGTTGGGCACTGACAATTCCGTGGTG

In certain embodiments, the sequence element to increase translation of transcripts is a transcriptional pause element. This “pause element” can regulate gene expression at the level of RNA synthesis in both prokaryotes and eukaryotes, serving to coordinate the appearance of RNA with its utilization in cellular functions, and to modulate the interaction of regulatory proteins with RNA polymerase (RNAP).

In certain embodiments, the pause element is a MAZ4 pause element. In certain embodiments, the MAZ4 pause element comprises a nucleotide sequence that is at least about 80%, about 85%, about 90%, about 95%, about 96%, about 97%, about 98%, or about 99% identical to the nucleotide sequence set forth in SEQ ID NO: 27. In certain embodiments, the MAZ4 pause element comprises the nucleotide sequence set forth in SEQ ID NO: 27. In certain embodiments, the MAZ4 pause element consists of the nucleotide sequence set forth in SEQ ID NO: 27. SEQ ID NO: 27 is provided below:

[SEQ ID NO: 27]
CCTGGCCTTGGGGGAGGGGGAGGCCAGAATGAGAGCTCCTGGCCTTGGGG
GAGGGGGAGGCCAGAATGACTCGACCTGGCCTTGGGGGAGGGGGAGGCCA
GAATGAGAGCTCCTGGCCTTGGGGGAGGGGGAGGCCAGAATGA

In certain embodiments, the dnTGFβRII Constructs described herein are designed to integrate into the genome in a manner such that the transcripts of the TCR and the payload (e.g., dnTGFβRII) include intronic regions (see, e.g., Format 1; FIGS. 13A, 13B, 14A, and 14C). In certain embodiments, the dnTGFβRII Constructs described herein are designed such that the transcript of the payload (e.g., dnTGFβRII) lacks intronic regions (see, e.g., Formats 2 and 3; FIGS. 15A-15C, 16A-16C, 17, and 18). In certain embodiments, in order to address the lack of intronic regions of the payload (e.g., dnTGFβRII) transcript (see, e.g., Formats 2 and 3; FIGS. 15A-15C, 16A-16C, 17, and 18), a sequence element that promotes translation of transcripts is introduced prior to the poly-A sequence in order to mediate proper processing prior to translation of the promoter transcript. In certain embodiments, the sequence element that promotes translation of transcripts is WPRE (see, e.g., Formats 2 and 3; FIGS. 15A, 16A, 17, and 18).

In certain embodiments, the WPRE element shown in FIGS. 15A, 16A, 17, and 18 as diagrams of Formats 2 and 3 can be substituted with wPRE3, HPRE, or wPRE-O.

In certain embodiments, the WPRE element shown in FIGS. 15A, 16A, 17, and 18 as diagrams of Formats 2 and 3 is wPRE3 (i.e., WPRE element shown in the figures is replaced with wPRE3). In certain embodiments, the WPRE element shown in FIGS. 15A, 16A, 17, and 18 as diagrams of Formats 2 and 3 is wPRE3 (i.e., WPRE element shown in the figures is replaced with wPRE3 as a preferred sequence element to promote translation of the transcript).

In certain embodiments, the promoter for the expression of the payload (e.g., dnTGFβRII) of the dnTGFβRII Construct is a eukaryotic promoter, mammalian promoter, viral promoter, synthetic promoter, minimal promoter hybrid promoter, tissue specific promoter, inducible promoter, or a constitutive promoter.

In certain embodiments, the promoter for the expression of the payload (e.g., dnTGFβRII) of the dnTGFβRII Construct is a constitutive promoter (i.e., a promoter that displays stable gene expression patterns over time). In certain embodiments, a constitutive promoter is selected in cases where the payload (e.g., dnTGFβRII) of interest has a low toxicity profile and in cases where there is no clear benefit in linking protein expression to a specific time or place. In certain non-limiting examples, the constitutive promoter is the EF-1α promoter, the hACTB promoter, the hPGK promoter, the MND promoter, or the U6 promoter.

In certain embodiments, the promoter for the expression of the payload (e.g., dnTGFβRII) of the dnTGFβRII Construct is an inducible promoter (i.e., a type of conditional promoter where the promoter is in its “on” state only under certain conditions). In certain embodiments, the inducible promoter is generally in its “off” state, unless and until it receives a signal that induces switching into the “on” state. In certain embodiments, the inducible promoter allows for a basal level of gene expression even if in the “off” state (i.e., a phenomenon known as promoter leakiness).

In certain embodiments, the inducible promoter is a TCR activation triggered inducible promoter. In certain embodiments, the TCR activation triggered inducible promoter works as follows: upon TCR engagement a transcription factor is activated (i.e. AP-1, NFAT, NF-κB); these activated transcription factors will bind the inducible promoter and initiate a switch from the “off” state to the “on” state. In certain embodiments, the TCR activation triggered inducible promoter may contain either additional or a reduced number of response elements when compared to the wild type promoter. In certain embodiments, the TCR activation triggered inducible promoter is used to localize a payload (e.g., dnTGFβRII) of interest to the tumor site. In certain embodiments, the benefit of localizing a payload (e.g., dnTGFβRII) of interest to the tumor site is to allow for high levels of the payload (e.g., dnTGFβRII) to be predominantly expressed in response to TCR signaling at the tumor site. In certain embodiments, this is the site maximal TCR engagement. In certain embodiments, localizing a payload of interest to the tumor site allows for the use of payloads with high toxicity profiles because the use of an inducible promoter can limit systemic availability of toxic payloads. In certain embodiments, the TCR activation triggered inducible promoter is the AP-1 responsive, NFAT responsive, or NF-κB responsive promoter.

In certain embodiments, the TCR activation triggered inducible promoter is a promoter that can be activated by pathways associated with the TCR activation or T cell pathways or by induction with an activation agent (for example but not limited to a small molecule or polypeptide).

In certain embodiments, the TCR activation triggered inducible promoter is the AP-1 responsive, NFAT responsive, NF-κB responsive promoter, or an NR4A-responsive promoter.

In certain embodiments, the promoter for the expression of the payload (e.g., dnTGFβRII) of the dnTGFβRII Construct is a TRAC promoter. In certain embodiments, the promoter for the expression of the payload (e.g., dnTGFβRII) of the dnTGFβRII Construct is a TCRβ promoter. In certain embodiments, the promoter for the expression of the payload (e.g., dnTGFβRII) of the dnTGFβRII Construct of Format 3 (FIG. 19) is the TRAC promoter. In certain embodiments, the promoter for the expression of the payload (e.g., dnTGFβRII) of the dnTGFβRII Construct of Format 3 (FIG. 19) is the TCRβ promoter.

In certain embodiments, the promoter for the expression of the payload (e.g., dnTGFβRII) of the dnTGFβRII Construct of Format 1 (FIGS. 13A, 13B, 14A, and 14C) is the EF1a core promoter. In certain embodiments, the promoter for the expression of the payload (e.g., dnTGFβRII) of the dnTGFβRII Construct of Format 1 (FIGS. 13A, 13B, 14A, and 14C) is the hACTB promoter. In certain embodiments, the promoter for the expression of the payload (e.g., dnTGFβRII) of the dnTGFβRII Construct of Format 1 (FIGS. 13A, 13B, 14A, and 14C) is the hPGK promoter. In certain embodiments, the promoter for the expression of the payload (e.g., dnTGFβRII) of the dnTGFβRII Construct of Format 1 (FIGS. 13A, 13B, 14A, and 14C) is the MND promoter. In certain embodiments, the promoter for the expression of the payload (e.g., dnTGFβRII) of the dnTGFβRII Construct of Format 1 (FIGS. 13A, 13B, 14A, and 14C) is the AP-1 responsive promoter. In certain embodiments, the promoter for the expression of the payload (e.g., dnTGFβRII) of the dnTGFβRII Construct of Format 1 (FIGS. 13A, 13B, 14A, and 14C) is the NFAT responsive promoter. In certain embodiments, the promoter for the expression of the payload (e.g., dnTGFβRII) of the dnTGFβRII Construct of Format 1 (FIGS. 13A, 13B, 14A, and 14C) is the NF-κB promoter.

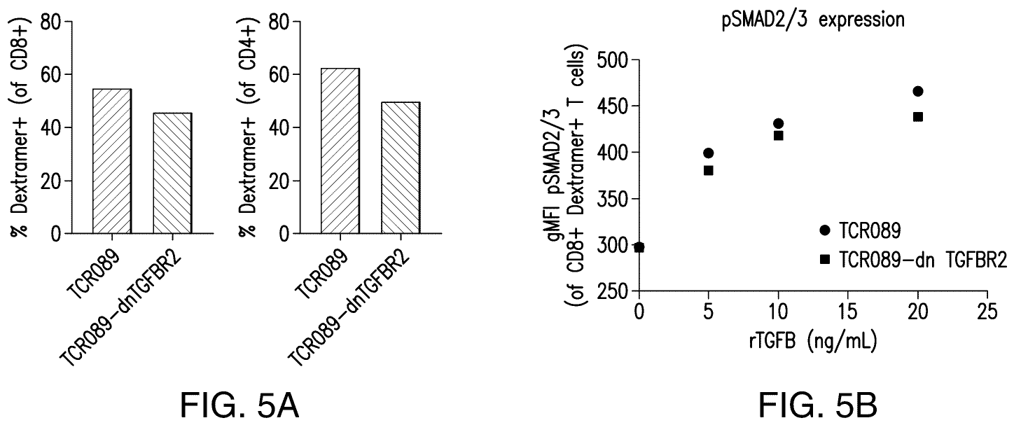

In certain embodiments, the promoter for the expression of the payload (e.g., dnTGFβRII) of the dnTGFβRII Construct of Format 2 (FIGS. 15A-15C and 16A-16C) is the EF1a core promoter. In certain embodiments, the promoter for the expression of the payload (e.g., dnTGFβRII) of the dnTGFβRII Construct of Format 2 (FIGS. 15A-15C and 16A-16C) is the hACTB promoter. In certain embodiments, the promoter for the expression of the payload (e.g., dnTGFβRII) of the dnTGFβRII Construct of Format 2 (FIGS. 15A-15C and 16A-16C) is the hPGK promoter. In certain embodiments, the promoter for the expression of the payload (e.g., dnTGFβRII) of the dnTGFβRII Construct of Format 2 (FIGS. 15A-15C and 16A-16C) is the MND promoter. In certain embodiments, the promoter for the expression of the payload (e.g., dnTGFβRII) of the dnTGFβRII Construct of Format 2 (FIGS. 15A-15C and 16A-16C) is the AP-1 responsive promoter. In certain embodiments, the promoter for the expression of the payload (e.g., dnTGFβRII) of the dnTGFβRII Construct of Format 2 (FIGS. 15A-15C and 16A-16C) is the NFAT responsive promoter. In certain embodiments, the promoter for the expression of the payload (e.g., dnTGFβRII) of the dnTGFβRII Construct of Format 2 (FIGS. 15A-15C and 5A-5C) is the NF-κB promoter.

In certain embodiments, the promoter for the expression of the payload (e.g., dnTGFβRII) of the dnTGFβRII Construct of Format 3 (FIGS. 17 and 18) is the EF1a core promoter. In certain embodiments, the promoter for the expression of the payload (e.g., dnTGFβRII) of the dnTGFβRII Construct of Format 3 (FIGS. 17 and 18) is the hACTB promoter. In certain embodiments, the promoter for the expression of the payload (e.g., dnTGFβRII) of the dnTGFβRII Construct of Format 3 (FIGS. 17 and 18) is the hPGK promoter. In certain embodiments, the promoter for the expression of the payload (e.g., dnTGFβRII) of the dnTGFβRII Construct of Format 3 (FIGS. 17 and 18) is the MND promoter. In certain embodiments, the promoter for the expression of the payload (e.g., dnTGFβRII) of the dnTGFβRII Construct of Format 3 (FIGS. 17 and 18) is the AP-1 responsive promoter. In certain embodiments, the promoter for the expression of the payload (e.g., dnTGFβRII) of the dnTGFβRII Construct of Format 3 (FIGS. 17 and 18) is the NFAT responsive promoter. In certain embodiments, the promoter for the expression of the payload (e.g., dnTGFβRII) of the dnTGFβRII Construct of Format 3 (FIGS. 17 and 18) is the NF-κB promoter.

In certain embodiments, the dnTGFβRII that is expressed by the dnTGFβRII Cells comprises the amino acid sequence set forth in SEQ ID NO: 8. In certain embodiments, dnTGFβRII that is expressed by the dnTGFβRII Cells comprises an amino acid sequence with one or more conservative substitutions of SEQ ID NO: 8.

In certain embodiments, the dnTGFβRII comprises an amino acid sequence that is at least about 80%, about 85%, about 90%, about 95%, about 96%, about 97%, about 98%, or about 99% identical to the amino acid sequence set forth in SEQ ID NO: 8. In certain embodiments, the dnTGFβRII comprises the amino acid sequence set forth in SEQ ID NO: 8. In certain embodiments, the dnTGFβRII consists of the amino acid sequence set forth in SEQ ID NO: 8. SEQ ID NO: 8 is provided below.

[SEQ ID NO: 8]
MGRGLLRGLWPLHIVLWTRIASTIPPHVQKSVNNDMIVTDNNGAVKFPQL
CKFCDVRFTCDNQKSCMSNCSITSICEKPQEVCVAVWRKNDENITLETVC
HDPKLPYHDFILEDAASPCIMKEKKKPGETFFMCSCSSDECNDNIIFSEE
YNTSNPDLLLVIFQVTGISLLPPLGVAISVIIIFYCYRVNRQQKLSSTWE
TGKTRKLMEFSEHCAIILEDDRSDISSTCANNINHNTELLPIELDTLVGK
GRFAEVYKAKLKQNTSEQFETVAVKIFPYEEYASWKTEKDIFSDINLKHE
NILQFLTAEERKTELGKQYWLITAFHAKGNLQEYLTRHVISWEDLRKLGS
SLARGIAHLHSDHTPCGRPKMPIVHRDLKSSNILVKNDLTCCLCDFGLSL
RLDPTLSVDDLANSGQVGTARYMAPEVLESRMNLENVESFKQTDVYSMAL
VLWEMTSRCNAVGEVKDYEPPFGSKVREHPCVESMKDNVLRDRGRPEIPS
FWLNHQGIQMVCETLTECWDHDPQARLTAQCVAERFSELEHLDRLSGRSC
SEEKIPEDGSLNTTK

In certain embodiments, the dnTGFβRII is encoded by the nucleotide sequence set forth in SEQ ID NO: 28 provided below.

[SEQ ID NO: 28]
ATGGGCCGCGGCCTGCTGCGCGGCCTGTGGCCGCTGCATATTGTGCTGTG
GACCCGCATTGCGAGCACCATTCCGCCGCATGTGCAGAAAAGCGTGAACA
ACGATATGATTGTGACCGATAACAACGGCGCGGTGAAATTTCCGCAGCTG
TGCAAATTTTGCGATGTGCGCTTTACCTGCGATAACCAGAAAAGCTGCAT
GAGCAACTGCAGCATTACCAGCATTTGCGAAAAACCGCAGGAAGTGTGCG
TGGCGGTGTGGCGCAAAAACGATGAAAACATTACCCTGGAAACCGTGTGC
CATGATCCGAAACTGCCGTATCATGATTTTATTCTGGAAGATGCGGCGAG
CCCGTGCATTATGAAAGAAAAAAAAAAACCGGGCGAAACCTTTTTTATGT
GCAGCTGCAGCAGCGATGAATGCAACGATAACATTATTTTTAGCGAAGAA
TATAACACCAGCAACCCGGATCTGCTGCTGGTGATTTTTCAGGTGACCGG
CATTAGCCTGCTGCCGCCGCTGGGCGTGGCGATTAGCGTGATTATTATTT
TTTATTGCTATCGCGTGAACCGCCAGCAGAAACTGAGCAGCACCTGGGAA
ACCGGCAAAACCCGCAAACTGATGGAATTTAGCGAACATTGCGCGATTAT
TCTGGAAGATGATCGCAGCGATATTAGCAGCACCTGCGCGAACAACATTA
ACCATAACACCGAACTGCTGCCGATTGAACTGGATACCCTGGTGGGCAAA
GGCCGCTTTGCGGAAGTGTATAAAGCGAAACTGAAACAGAACACCAGCGA
ACAGTTTGAAACCGTGGCGGTGAAAATTTTTCCGTATGAAGAATATGCGA
GCTGGAAAACCGAAAAAGATATTTTTAGCGATATTAACCTGAAACATGAA
AACATTCTGCAGTTTCTGACCGCGGAAGAACGCAAAACCGAACTGGGCAA
ACAGTATTGGCTGATTACCGCGTTTCATGCGAAAGGCAACCTGCAGGAAT
ATCTGACCCGCCATGTGATTAGCTGGGAAGATCTGCGCAAACTGGGCAGC
AGCCTGGCGCGCGGCATTGCGCATCTGCATAGCGATCATACCCCGTGCGG
CCGCCCGAAAATGCCGATTGTGCATCGCGATCTGAAAAGCAGCAACATTC
TGGTGAAAAACGATCTGACCTGCTGCCTGTGCGATTTTGGCCTGAGCCTG
CGCCTGGATCCGACCCTGAGCGTGGATGATCTGGCGAACAGCGGCCAGGT
GGGCACCGCGCGCTATATGGCGCCGGAAGTGCTGGAAAGCCGCATGAACC
TGGAAAACGTGGAAAGCTTTAAACAGACCGATGTGTATAGCATGGCGCTG
GTGCTGTGGGAAATGACCAGCCGCTGCAACGCGGTGGGCGAAGTGAAAGA
TTATGAACCGCCGTTTGGCAGCAAAGTGCGCGAACATCCGTGCGTGGAAA
GCATGAAAGATAACGTGCTGCGCGATCGCGGCCGCCCGGAAATTCCGAGC
TTTTGGCTGAACCATCAGGGCATTCAGATGGTGTGCGAAACCCTGACCGA
ATGCTGGGATCATGATCCGCAGGCGCGCCTGACCGCGCAGTGCGTGGCGG
AACGCTTTAGCGAACTGGAACATCTGGATCGCCTGAGCGGCCGCAGCTGC
AGCGAAGAAAAAATTCCGGAAGATGGCAGCCTGAACACCACCAAATAA

In certain embodiments, the dnTGFβRII comprises an amino acid sequence that is at least about 80%, about 85%, about 90%, about 95%, about 96%, about 97%, about 98%, or about 99% identical to the amino acid sequence set forth in SEQ ID NO: 38. In certain embodiments, the dnTGFβRII comprises the amino acid sequence set forth in SEQ ID NO: 38. In certain embodiments, the dnTGFβRII consists of the amino acid sequence set forth in SEQ ID NO: 38. SEQ ID NO: 38 is provided below.

[SEQ ID NO: 38]
MGRGLLRGLWPLHIVLWTRIASTIPPHVQKSVNNDMIVTDNNGAVKFPQL
CKFCDVRFTCDNQKSCMSNCSITSICEKPQEVCVAVWRKNDENITLETVC
HDPKLPYHDFILEDAASPCIMKEKKKPGETFFMCSCSSDECNDNIIFSEE
YNTSNPDLLLVIFQVTGISLLPPLGVAISVIIIFYCYRVNRQQKLS

In certain embodiments, the dnTGFβRII is encoded by the nucleotide sequence set forth in SEQ ID NO: 39 provided below.

[SEQ ID NO: 39]
ATGGGACGCGGGCTGTTGCGGGGTCTCTGGCCACTGCATATCGTGCTTTG
GACCAGGATCGCATCTACAATTCCGCCGCACGTACAAAAGTCAGATGTGG
AGATGGAGGCCCAAAAAGACGAAATTATTTGCCCTTCTTGTAACAGGACA
GCTCACCCACTTCGCCACATCAACAATGACATGATAGTGACCGACAATAA
CGGAGCAGTAAAATTCCCTCAGTTGTGCAAGTTCTGTGACGTGCGATTCA
GCACTTGTGATAACCAGAAGTCCTGTATGTCTAATTGTTCCATCACGTCT
ATTTGCGAAAAACCGCAAGAAGTCTGCGTCGCGGTATGGCGGAAGAACGA
CGAAAATATTACGCTTGAGACAGTTTGTCACGATCCAAAACTTCCGTACC
ATGACTTCATTCTTGAGGATGCGGCAAGCCCCAAGTGCATTATGAAGGAG
AAGAAGAAACCTGGAGAAACCTTCTTCATGTGCAGTTGTTCCTCCGACGA
ATGTAATGACAACATTATTTTTTCAGAGGAATATAATACCTCCAATCCAG
ACTTGTTGCTGGTAATCTTCCAAGTGACGGGAATCAGTTTGCTTCCGCCT
CTTGGGGTAGCCATCTCAGTTATAATTATCTTTTACTGCTATCGCGTTAA
CCGGCAACAAAAATTGAGT

In certain embodiments, the dnTGFβRII Products comprise dnTGFβRII Cells that have a greater proliferative capacity than aNeoTCR Product expressing the same NeoTCR that does not express a dnTGFβRII.

In certain embodiments, the dnTGFβRII Products comprise dnTGFβRII Cells that can proliferate in vitro for a period of time up to about two (2) weeks. In certain embodiments, the dnTGFβRII Products comprise dnTGFβRII Cells that proliferate in vitro for a period of time between about two (2) weeks and about one (1) month. In certain embodiments, the dnTGFβRII Products comprise dnTGFβRII Cells that can proliferate in vitro for a period of time between about one (1) month and about two (2) months. In certain embodiments, the dnTGFβRII Products comprise dnTGFβRII Cells that can proliferate in vitro for a period of time between about two (2) months and about three (3) months. In certain embodiments, the dnTGFβRII Products comprise dnTGFβRII Cells that can proliferate in vitro for a period of time between about three (3) months and about four (4) months. In certain embodiments, the dnTGFβRII Products comprise dnTGFβRII Cells that can proliferate in vitro for a period of time between about four (4) months and about five (5) months. In certain embodiments, the dnTGFβRII Products comprise dnTGFβRII Cells that proliferate in vitro for a period of time between about five (5) months and about six (6) months. In certain embodiments, the dnTGFβRII Products comprise dnTGFβRII Cells that can proliferate in vitro for a period of time greater than about six (6) months.

In certain embodiments, the dnTGFβRII Products comprise dnTGFβRII Cells that can proliferate ex vivo for a period of time up to about two (2) weeks. In certain embodiments, the dnTGFβRII Products comprise dnTGFβRII Cells that proliferate ex vivo for a period of time between about two (2) weeks and about one (1) month. In certain embodiments, the dnTGFβRII Products comprise dnTGFβRII Cells that can proliferate ex vivo for a period of time between about one (1) month and about two (2) months. In certain embodiments, the dnTGFβRII Products comprise dnTGFβRII Cells that can proliferate ex vivo for a period of time between about two (2) months and about three (3) months. In certain embodiments, the dnTGFβRII Products comprise dnTGFβRII Cells that can proliferate ex vivo for a period of time between about three (3) months and about four (4) months. In certain embodiments, the dnTGFβRII Products comprise dnTGFβRII Cells that can proliferate ex vivo for a period of time between about four (4) months and about five (5) months. In certain embodiments, the dnTGFβRII Products comprise dnTGFβRII Cells that proliferate ex vivo for a period of time between about five (5) months and about six (6) months. In certain embodiments, the dnTGFβRII Products comprise dnTGFβRII Cells that can proliferate ex vivo for a period of time greater than about six (6) months.

In certain embodiments, the dnTGFβRII Products comprise dnTGFβRII Cells that can proliferate in vivo for a period of time up to about two (2) weeks. In certain embodiments, the dnTGFβRII Products comprise dnTGFβRII Cells that can proliferate in vivo for a period of time between about two (2) weeks and about one (1) month. In certain embodiments, the dnTGFβRII Products comprise dnTGFβRII Cells that can proliferate in vivo for a period of time between about one (1) month and about two (2) months. In certain embodiments, the dnTGFβRII Products comprise dnTGFβRII Cells that can proliferate in vivo for a period of time between about two (2) months and about three (3) months. In certain embodiments, the dnTGFβRII Products comprise dnTGFβRII Cells that can proliferate in vivo for a period of time between about three (3) months and about four (4) months. In certain embodiments, the dnTGFβRII Products comprise dnTGFβRII Cells that can proliferate in vivo for a period of time between about four (4) months and about five (5) months. In certain embodiments, the dnTGFβRII Products comprise dnTGFβRII Cells that can proliferate in vivo for a period of time between about five (5) months and about six (6) months. In certain embodiments, the dnTGFβRII Products comprise dnTGFβRII Cells that can proliferate in vivo for a period of time for greater than about six (6) months.

In certain embodiments, the dnTGFβRII Products comprise dnTGFβRII Cells that can proliferate in vivo as long as there are tumor cells expressing the neoantigen cognate to the NeoTCR expressed by the dnTGFβRII Cells. In certain embodiments, the dnTGFβRII Products comprise dnTGFβRII Cells that can proliferate in vivo as long as there are cells expressing the neoantigen cognate to the NeoTCR expressed by the dnTGFβRII Cells. In certain embodiments, the dnTGFβRII Products comprise dnTGFβRII Cells that can only proliferate in vivo in the presence of a cell that expresses a neoantigen cognate to the NeoTCR expressed by the dnTGFβRII Cells. In certain embodiments, in vivo means in a patient following infusion of a dnTGFβRII Product.

In certain embodiments, the dnTGFβRII Products comprise dnTGFβRII Cells that can persist longer in patient's post-infusion than a NeoTCR Product expressing the same NeoTCR and that does not comprise a dnTGFβRII knockout or substantial knockdown thereof.

In certain embodiments, the dnTGFβRII Products comprise dnTGFβRII Cells that can persist in vitro for a period of time up to about two (2) weeks. In certain embodiments, the dnTGFβRII Products comprise dnTGFβRII Cells that can persist in vitro for a period of time between about two (2) weeks and about one (1) month. In certain embodiments, the dnTGFβRII Products comprise dnTGFβRII Cells that can persist in vitro for a period of time between about one (1) month and about two (2) months. In certain embodiments, the dnTGFβRII Products comprise dnTGFβRII Cells that can persist in vitro for a period of time between about two (2) months and about three (3) months. In certain embodiments, the dnTGFβRII Products comprise dnTGFβRII Cells that can persist in vitro for a period of time between about three (3) months and about four (4) months. In certain embodiments, the dnTGFβRII Products comprise dnTGFβRII Cells that can persist in vitro for a period of time between about four (4) months and about five (5) months. In certain embodiments, the dnTGFβRII Products comprise dnTGFβRII Cells that can persist in vitro for a period of time between about five (5) months and about six (6) months. In certain embodiments, the dnTGFβRII Products comprise dnTGFβRII Cells that can persist in vitro for a period of time greater than about six (6) months.

In certain embodiments, the dnTGFβRII Products comprise dnTGFβRII Cells that can persist ex vivo for a period of time up to about two (2) weeks. In certain embodiments, the dnTGFβRII Products comprise dnTGFβRII Cells that can persist ex vivo for a period of time between about two (2) weeks and about one (1) month. In certain embodiments, the dnTGFβRII Products comprise dnTGFβRII Cells that can persist ex vivo for a period of time between about one (1) month and about two (2) months. In certain embodiments, the dnTGFβRII Products comprise dnTGFβRII Cells that can persist ex vivo for a period of time between about two (2) months and about three (3) months. In certain embodiments, the dnTGFβRII Products comprise dnTGFβRII Cells that can persist ex vivo for a period of time between about three (3) months and about four (4) months. In certain embodiments, the dnTGFβRII Products comprise dnTGFβRII Cells that can persist ex vivo for a period of time between about four (4) months and about five (5) months. In certain embodiments, the dnTGFβRII Products comprise dnTGFβRII Cells that can persist ex vivo for a period of time between about five (5) months and about six (6) months. In certain embodiments, the dnTGFβRII Products comprise dnTGFβRII Cells that can persist ex vivo for a period of time greater than about six (6) months.

In certain embodiments, the dnTGFβRII Products comprise dnTGFβRII Cells that can persist in vivo for a period of time up to about two (2) weeks. In certain embodiments, the dnTGFβRII Products comprise dnTGFβRII Cells that can persist in vivo for a period of time between about two (2) weeks and about one (1) month. In certain embodiments, the dnTGFβRII Products comprise dnTGFβRII Cells that can persist in vivo for a period of time between about one (1) month and about two (2) months. In certain embodiments, the dnTGFβRII Products comprise dnTGFβRII Cells that can persist in vivo for a period of time between about two (2) months and about three (3) months. In certain embodiments, the dnTGFβRII Products comprise dnTGFβRII Cells that can persist in vivo for a period of time between about three (3) months and about four (4) months. In certain embodiments, the dnTGFβRII Products comprise dnTGFβRII Cells that can persist in vivo for a period of time between about four (4) months and about five (5) months. In certain embodiments, the dnTGFβRII Products comprise dnTGFβRII Cells that can persist in vivo for a period of time between about five (5) months and about six (6) months. In certain embodiments, the dnTGFβRII Products comprise dnTGFβRII Cells that can persist in vivo for a period of time greater than about six (6) months.

In certain embodiments, the dnTGFβRII Products comprise dnTGFβRII Cells that can persist in vivo as long as there are tumor cells expressing the neoantigen cognate to the NeoTCR expressed by the dnTGFβRII Cells. In certain embodiments, the dnTGFβRII Products comprise dnTGFβRII Cells that can persist in vivo as long as there are cells expressing the neoantigen cognate to the NeoTCR expressed by the dnTGFβRII Cells. In certain embodiments, the dnTGFβRII Products comprise dnTGFβRII Cells that can only persist in vivo in the presence of a cell that expresses a neoantigen cognate to the NeoTCR expressed by the dnTGFβRII Cells. In certain embodiments, in vivo means in a patient following infusion of a dnTGFβRII Product.

In certain embodiments, dnTGFβRII Cells are expanded in vitro or ex vivo in a manner that preserves a “young” T cell phenotypes, resulting in a dnTGFβRII Product in which the majority of the T cells exhibit T memory stem cell and T central memory phenotypes.

### 2.3. CD8-TGFβRII Products

In certain embodiments, the present disclosure provides NeoTCR Cells including a knockout of a TGFβR locus and an exogenous CD8 receptor.

In certain embodiments, the NeoTCR Cells described herein are CD8-TGFβRII Cells. In certain embodiments, the NeoTCR Products described herein are CD8-TGFβRII Products.

In certain embodiments, the NeoTCR Cells described above can be further modified to knockout or substantially knockdown TGFβRII expression from the cells and knock in a CD8 Construct (i.e., a CD8-TGFβRII Product). Specifically, using the gene editing technology and NeoTCR isolation technology described in PCT/US2020/017887 and PCT/US2019/025415, which are incorporated herein in their entireties, NeoTCRs were cloned in autologous CD8⁺ and CD4⁺ T cells from the patient of origin with cancer by precision genome engineered (using a DNA-mediated (non-viral) method) to express the NeoTCR while concurrently knocking in a CD8 Construct and knocking out or substantially knockdown the endogenous expression of TGFβRII in the CD8⁺ and CD4⁺ T cells.

In certain embodiments, the CD8-TGFβRII Cells include a gene disruption of the TGFβRII locus that generates a knockout of the TGFβRII gene expression. In certain embodiments, the CD8-TGFβRII Cells include a gene disruption of the TGFβRII locus that generates a knockdown of the TGFβRII gene expression. In certain embodiments, the gene disruption of the TGFβRII locus can be obtained by any of the gene editing methods disclosed in Section 2.5 below. In certain non-limiting embodiments, the gene disruption is obtained by homologous recombination, non-homologous end joining, targeted nucleases (e.g., endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease).

In certain embodiments, the gene disruption of the TGFβRII locus of the CD8-TGFβRII Cells can be a disruption of the coding region of the TGFβRII locus (e.g., exons). In certain embodiments, the gene disruption of the TGFβRII locus of the CD8-TGFβRII Cells can be a disruption of the non-coding region of the TGFβRII locus (e.g., introns). Human TGFβRII locus includes seven exons: exon 1, exon 2, exon 3, exon 4, exon 5, exon 6, and exon 7. In certain embodiments, the gene disruption of the TGFβRII locus can be a disruption of the exon 1 of the TGFβRII locus of the CD8-TGFβRII Cells. In certain embodiments, the gene disruption of the TGFβRII locus can be a disruption of the exon 2 of the TGFβRII locus of the CD8-TGFβRII Cells.

In certain embodiments, the TGFβRII gene is disrupted using a CRISPR/Cas nuclease system. In certain embodiments, the TGFβRII gene of a CD8-TGFβRII Cell is disrupted using a gRNA to knockout expression of TGFβRII in the same reaction as the reaction used to knockout the TCRβ and insertion of the NeoTCRα and NeoTCRβ (see FIG. 4). In certain embodiments, the knockout the TCRβ, insertion of the NeoTCRα and NeoTCRβ, and knockout of TGFβRII are performed concurrently in the same reaction to produce a CD8-TGFβRII Cell.

In certain embodiments, the TGFβRII gene of a CD8-TGFβRII Cell is disrupted using a gRNA to knockout expression of TGFβRII in a different reaction as the reaction used to knockout the TCRβ and insertion of the NeoTCRα and NeoTCRβ (see FIG. 4). In certain embodiments, the knockout of the TCRβ and insertion of the NeoTCRα and NeoTCRβ are performed in separate and consecutive reactions from the knockout of TGFβRII to produce a CD8-TGFβRII Cell. In certain embodiments, the knockout of the TCRβ and insertion of the NeoTCRα and NeoTCRβ are performed in a first reaction and the knockout of TGFβRII is performed in a second reaction to produce a CD8-TGFβRII Cell. In certain embodiments, the knockout of TGFβRII is performed in a first reaction and the knockout of the TCRβ and insertion of the NeoTCRα and NeoTCRβ are performed in a second reaction to produce a CD8-TGFβRII Cell.

In certain embodiments, the CD8-TGFβRII Products comprise cells that are engineered to express one or more NeoTCRs using viral methods (i.e., NeoTCR Viral Product) and a knockout of the TGFβRII gene. In certain embodiments, the cells of the NeoTCR Viral Product are further engineered to knockout the TGFβRII gene using non-viral methods. In certain embodiments, the cells of the NeoTCR Viral Product are further engineered to knockout the TGFβRII gene using viral methods.

In certain embodiments, the CD8-TGFβRII Product manufacturing process involves electroporation of 1) dual ribonucleoprotein species of CRISPR-Cas9 nucleases bound to guide RNA sequences, with each species targeting the genomic TCRα and the genomic TCRβ loci, and 2) ribonucleoprotein species of CRISPR-Cas9 nucleases bound to guide RNA sequences that target the TGFβRII gene. The specificity of targeting Cas9 nucleases to each genomic locus has been previously described in the literature as being highly specific. Comprehensive testing of the CD8-TGFβRII Product can be performed in vitro and in silico analyses to survey possible off-target genomic cleavage sites, using COSMID and GUIDE-seq, respectively. In certain embodiments, testing can be performed using COSMID-based in silico prediction of off-targets and GUIDE-seq-based in vitro prediction of off-targets, followed by testing of those putative off-targets by targeted deep sequencing.

In certain embodiments, the CD8-TGFβRII Products comprise CD8-TGFβRII Cells wherein the endogenous TGFβRII is knocked out. In certain embodiments, the CD8-TGFβRII Products comprise TGFβRII Cells wherein the endogenous TGFβRII is substantially knocked down.

In certain embodiments, the CD8-TGFβRII Products comprise TGFβRII Cells wherein the endogenous TGFβRII is knocked out using a CRISPR system. In certain embodiments, the CD8-TGFβRII Products comprise CD8-TGFβRII Cells wherein the endogenous TGFβRII is knocked out using the CRISPR/Cas9 system. In certain embodiments, the CD8-TGFβRII Products comprise CD8-TGFβRII Cells wherein the endogenous TGFβRII is substantially knocked down using a CRISPR system. In certain embodiments, the CD8-TGFβRII Products comprise CD8-TGFβRII Cells wherein the endogenous TGFβRII is substantially knocked down using the CRISPR/Cas9 system. In certain embodiments, the CD8-TGFβRII Products comprise CD8-TGFβRII Cells wherein the endogenous TGFβRII is substantially knocked down using shRNA.

In certain embodiments, the CD8-TGFβRII Products comprise CD8-TGFβRII Cells wherein the endogenous TGFβRII is knocked out using the CRISPR/Cas9 system with the gRNAs provided in Table 1. In certain embodiments, the CD8-TGFβRII Products comprise CD8-TGFβRII Cells wherein the endogenous TGFβRII is substantially knocked down using the CRISPR/Cas9 system with the gRNAs provided in Table 1.

In certain embodiments, the CD8-TGFβRII Products comprise CD8-TGFβRII Cells that have a greater proliferative capacity than a NeoTCR Product expressing the same NeoTCR that does not comprise a TGFβRII knockout or substantial knockdown and/or does not comprise a CD8 Construct knock in. In certain embodiments, the CD8-TGFβRII Products comprise CD8-TGFβRII Cells that have a greater proliferative capacity than a NeoTCR Product expressing the same NeoTCR that does not comprise a TGFβRII knockout or substantial knockdown. In certain embodiments, the CD8-TGFβRII Products comprise CD8-TGFβRII Cells that have a greater proliferative capacity than a NeoTCR Product expressing the same NeoTCR that does not comprise a CD8 Construct knock in. In certain embodiments, the CD8-TGFβRII Products comprise CD8-TGFβRII Cells that have a greater proliferative capacity than a CD8 Product expressing the same NeoTCR that does not comprise a TGFβRII knockout or substantial knockdown. In certain embodiments, the CD8-TGFβRII Products comprise CD8-TGFβRII Cells that have a greater proliferative capacity than a CD8 Product expressing the same NeoTCR that does not comprise a TGFβRII knockout or substantial knockdown and a CD8 Construct knock in. In certain embodiments, the CD8-TGFβRII Products comprise CD8-TGFβRII Cells that have a greater proliferative capacity than a dnTGFβRII Product expressing the same NeoTCR.

In certain embodiments, the CD8-TGFβRII Cells are capable of proliferating at a higher rate as compared to NeoTCR Cells. In certain embodiments, the CD8-TGFβRII Cells are capable of proliferating at a higher rate as compared to TGFβRII Cells. In certain embodiments, the CD8-TGFβRII Cells are capable of proliferating at a higher rate as compared to dnTGFβRII Cells. In certain embodiments, the CD8-TGFβRII Cells are capable of proliferating at a higher rate as compared to CD8 Cells.

In certain embodiments, the CD8-TGFβRII Cells are capable of proliferating for longer periods of time as compared to NeoTCR Cells. In certain embodiments, the CD8-TGFβRII Cells are capable of proliferating for longer periods of time as compared to TGFβRII Cells. In certain embodiments, the CD8-TGFβRII Cells are capable of proliferating for longer periods of time as compared to dnTGFβRII Cells. In certain embodiments, the CD8-TGFβRII Cells are capable of proliferating for longer periods of time as compared to CD8 Cells.

In certain embodiments, the CD8-TGFβRII Products comprise CD8-TGFβRII Cells that can proliferate in vitro for a period of time up to about two (2) weeks. In certain embodiments, the CD8-TGFβRII Products comprise CD8-TGFβRII Cells that proliferate in vitro for a period of time between about two (2) weeks and about one (1) month. In certain embodiments, the CD8-TGFβRII Products comprise CD8-TGFβRII Cells that can proliferate in vitro for a period of time between about one (1) month and about two (2) months. In certain embodiments, the CD8-TGFβRII Products comprise CD8-TGFβRII Cells that can proliferate in vitro for a period of time between about two (2) months and about three (3) months. In certain embodiments, the CD8-TGFβRII Products comprise CD8-TGFβRII Cells that can proliferate in vitro for a period of time between about three (3) months and about four (4) months. In certain embodiments, the CD8-TGFβRII Products comprise CD8-TGFβRII Cells that can proliferate in vitro for a period of time between about four (4) months and about five (5) months. In certain embodiments, the CD8-TGFβRII Products comprise CD8-TGFβRII Cells that proliferate in vitro for a period of time between about five (5) months and about six (6) months. In certain embodiments, the CD8-TGFβRII Products comprise CD8-TGFβRII Cells that can proliferate in vitro for a period of time greater than about six (6) months.

In certain embodiments, the CD8-TGFβRII Products comprise CD8-TGFβRII Cells that can proliferate ex vivo for a period of time up to about two (2) weeks. In certain embodiments, the CD8-TGFβRII Products comprise CD8-TGFβRII Cells that proliferate ex vivo for a period of time between about two (2) weeks and about one (1) month. In certain embodiments, the CD8-TGFβRII Products comprise CD8-TGFβRII Cells that can proliferate ex vivo for a period of time between about one (1) month and about two (2) months. In certain embodiments, the CD8-TGFβRII Products comprise CD8-TGFβRII Cells that can proliferate ex vivo for a period of time between about two (2) months and about three (3) months. In certain embodiments, the CD8-TGFβRII Products comprise CD8-TGFβRII Cells that can proliferate ex vivo for a period of time between about three (3) months and about four (4) months. In certain embodiments, the CD8-TGFβRII Products comprise CD8-TGFβRII Cells that can proliferate ex vivo for a period of time between about four (4) months and about five (5) months. In certain embodiments, the CD8-TGFβRII Products comprise CD8-TGFβRII Cells that proliferate ex vivo for a period of time between about five (5) months and about six (6) months. In certain embodiments, the CD8-TGFβRII Products comprise CD8-TGFβRII Cells that can proliferate ex vivo for a period of time greater than about six (6) months.

In certain embodiments, the CD8-TGFβRII Products comprise CD8-TGFβRII Cells that can proliferate in vivo for a period of time up to about two (2) weeks. In certain embodiments, the CD8-TGFβRII Products comprise CD8-TGFβRII Cells that can proliferate in vivo for a period of time between about two (2) weeks and about one (1) month. In certain embodiments, the CD8-TGFβRII Products comprise CD8-TGFβRII Cells that can proliferate in vivo for a period of time between about one (1) month and about two (2) months. In certain embodiments, the CD8-TGFβRII Products comprise CD8-TGFβRII Cells that can proliferate in vivo for a period of time between about two (2) months and about three (3) months. In certain embodiments, the CD8-TGFβRII Products comprise CD8-TGFβRII Cells that can proliferate in vivo for aperiod of time between about three (3) months and about four (4) months. In certain embodiments, the CD8-TGFβRII Products comprise CD8-TGFβRII Cells that can proliferate in vivo for a period of time between about four (4) months and about five (5) months. In certain embodiments, the CD8-TGFβRII Products comprise CD8-TGFβRII Cells that can proliferate in vivo for a period of time between about five (5) months and about six (6) months. In certain embodiments, the CD8-TGFβRII Products comprise CD8-TGFβRII Cells that can proliferate in vivo for a period of time for greater than about six (6) months.

In certain embodiments, the CD8-TGFβRII Products comprise CD8-TGFβRII Cells that can proliferate in vivo as long as there are tumor cells expressing the neoantigen cognate to the NeoTCR expressed by the CD8-TGFβRII Cells. In certain embodiments, the CD8-TGFβRII Products comprise CD8-TGFβRII Cells that can proliferate in vivo as long as there are cells expressing the neoantigen cognate to the NeoTCR expressed by the CD8-TGFβRII Cells. In certain embodiments, the CD8-TGFβRII Products comprise CD8-TGFβRII Cells that can only proliferate in vivo in the presence of a cell that expresses a neoantigen cognate to the NeoTCR expressed by the CD8-TGFβRII Cells. In certain embodiments, in vivo means in a patient following infusion of a CD8-TGFβRII Product.

In certain embodiments, the CD8-TGFβRII Products comprise CD8-TGFβRII Cells that persist longer than a NeoTCR Product expressing the same NeoTCR that does not comprise a TGFβRII knockout or substantial knockdown and/or a CD8 Construct knock in. In certain embodiments, the CD8-TGFβRII Products comprise CD8-TGFβRII Cells that persist longer than a NeoTCR Product expressing the same NeoTCR that does not comprise a TGFβRII knockout or substantial knockdown. In certain embodiments, the CD8-TGFβRII Products comprise CD8-TGFβRII Cells that persist longer than a NeoTCR Product expressing the same NeoTCR that does not comprise a CD8 Construct knock in. In certain embodiments, the CD8-TGFβRII Products comprise CD8-TGFβRII Cells that persist longer than a CD8 Product expressing the same NeoTCR that does not comprise a TGFβRII knockout or substantial knockdown. In certain embodiments, the CD8-TGFβRII Products comprise CD8-TGFβRII Cells that persist longer than a CD8 Product expressing the same NeoTCR that does not comprise a TGFβRII knockout or substantial knockdown and a CD8 Construct knock in. In certain embodiments, the CD8-TGFβRII Products comprise CD8-TGFβRII Cells that persist longer than a dnTGFβRII Product expressing the same NeoTCR. In certain embodiments, the longer persistence is in vitro. In certain embodiments, the longer persistence is in vivo. In certain embodiments, the longer persistence in vivo is in a patient following infusion of a CD8-TGFβRII Product.

In certain embodiments, the CD8-TGFβRII Cells are capable of persisting longer than NeoTCR Cells. In certain embodiments, the CD8-TGFβRII Cells are capable of persisting longer than TGFβRII Cells. In certain embodiments, the CD8-TGFβRII Cells are capable of persisting longer than dnTGFβRII Cells. In certain embodiments, the CD8-TGFβRII Cells are capable of persisting longer than CD8 Cells.

In certain embodiments, the CD8-TGFβRII Cells are capable of persisting for longer periods of time as compared to NeoTCR Cells. In certain embodiments, the CD8-TGFβRII Cells are capable of persisting for longer periods of time as compared to TGFβRII Cells. In certain embodiments, the CD8-TGFβRII Cells are capable of persisting for longer periods of time as compared to dnTGFβRII Cells. In certain embodiments, the CD8-TGFβRII Cells are capable of persisting for longer periods of time as compared to CD8 Cells.

In certain embodiments, the CD8-TGFβRII Products comprise CD8-TGFβRII Cells that can persist in vitro for a period of time up to about two (2) weeks. In certain embodiments, the CD8-TGFβRII Products comprise CD8-TGFβRII Cells that can persist in vitro for a period of time between about two (2) weeks and about one (1) month. In certain embodiments, the CD8-TGFβRII Products comprise CD8-TGFβRII Cells that can persist in vitro for a period of time between about one (1) month and about two (2) months. In certain embodiments, the CD8-TGFβRII Products comprise CD8-TGFβRII Cells that can persist in vitro for a period of time between about two (2) months and about three (3) months. In certain embodiments, the CD8-TGFβRII Products comprise CD8-TGFβRII Cells that can persist in vitro for a period of time between about three (3) months and about four (4) months. In certain embodiments, the CD8-TGFβRII Products comprise CD8-TGFβRII Cells that can persist in vitro for a period of time between about four (4) months and about five (5) months. In certain embodiments, the CD8-TGFβRII Products comprise CD8-TGFβRII Cells that can persist in vitro for a period of time between about five (5) months and about six (6) months. In certain embodiments, the CD8-TGFβRII Products comprise CD8-TGFβRII Cells that can persist in vitro for a period of time greater than about six (6) months.

In certain embodiments, the CD8-TGFβRII Products comprise CD8-TGFβRII Cells that can persist ex vivo for a period of time up to about two (2) weeks. In certain embodiments, the CD8-TGFβRII Products comprise CD8-TGFβRII Cells that can persist ex vivo for a period of time between about two (2) weeks and about one (1) month. In certain embodiments, the CD8-TGFβRII Products comprise CD8-TGFβRII Cells that can persist ex vivo for a period of time between about one (1) month and about two (2) months. In certain embodiments, the CD8-TGFβRII Products comprise CD8-TGFβRII Cells that can persist ex vivo for a period of time between about two (2) months and about three (3) months. In certain embodiments, the CD8-TGFβRII Products comprise CD8-TGFβRII Cells that can persist ex vivo for a period of time between about three (3) months and about four (4) months. In certain embodiments, the CD8-TGFβRII Products comprise CD8-TGFβRII Cells that can persist ex vivo for a period of time between about four (4) months and about five (5) months. In certain embodiments, the CD8-TGFβRII Products comprise CD8-TGFβRII Cells that can persist ex vivo for a period of time between about five (5) months and about six (6) months. In certain embodiments, the CD8-TGFβRII Products comprise CD8-TGFβRII Cells that can persist ex vivo for a period of time greater than about six (6) months.

In certain embodiments, the CD8-TGFβRII Products comprise CD8-TGFβRII Cells that can persist in vivo for a period of time up to about two (2) weeks. In certain embodiments, the CD8-TGFβRII Products comprise CD8-TGFβRII Cells that can persist in vivo for a period of time between about two (2) weeks and about one (1) month. In certain embodiments, the CD8-TGFβRII Products comprise CD8-TGFβRII Cells that can persist in vivo for a period of time between about one (1) month and about two (2) months. In certain embodiments, the CD8-TGFβRII Products comprise CD8-TGFβRII Cells that can persist in vivo for a period of time between about two (2) months and about three (3) months. In certain embodiments, the CD8-TGFβRII Products comprise CD8-TGFβRII Cells that can persist in vivo for a period of time between about three (3) months and about four (4) months. In certain embodiments, the CD8-TGFβRII Products comprise CD8-TGFβRII Cells that can persist in vivo for a period of time between about four (4) months and about five (5) months. In certain embodiments, the CD8-TGFβRII Products comprise CD8-TGFβRII Cells that can persist in vivo for a period of time between about five (5) months and about six (6) months. In certain embodiments, the CD8-TGFβRII Products comprise CD8-TGFβRII Cells that can persist in vivo for a period of time greater than about six (6) months.

In certain embodiments, the CD8-TGFβRII Products comprise CD8-TGFβRII Cells that can persist in vivo as long as there are tumor cells expressing the neoantigen cognate to the NeoTCR expressed by the CD8-TGFβRII Cells. In certain embodiments, the T CD8-TGFβRII GFβRII Products comprise CD8-TGFβRII Cells that can persist in vivo as long as there are cells expressing the neoantigen cognate to the NeoTCR expressed by the CD8-TGFβRII Cells. In certain embodiments, the CD8-TGFβRII Products comprise CD8-TGFβRII Cells that can only persist in vivo in the presence of a cell that expresses a neoantigen cognate to the NeoTCR expressed by the CD8-TGFβRII Cells. In certain embodiments, in vivo means in a patient following infusion of a CD8-TGFβRII Product.

In certain embodiments, CD8-TGFβRII Cells are expanded in vitro or ex vivo in a manner that preserves “young” T cell phenotypes, resulting in a CD8-TGFβRII Product in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of TGFβRII Product including significant amounts of ‘young’ T cell, has the potential to benefit patients with cancer, through improved engraftment potential, prolonged persistence post-infusion, and rapid differentiation into effector T cells to eradicate tumor cells throughout the body.

In certain embodiments, the CD8-TGFβRII cells of the CD8-TGFβRII Products predominantly comprise memory stem cells (T_{msc}) and/or central memory cells (T_{cm}). In certain embodiments, at least about 25% of the CD8-TGFβRII cells of the CD8-TGFβRII Products comprise memory stem cells (T_{msc}) and/or central memory cells (Tₑₘ). In certain embodiments, at least about 30% of the CD8-TGFβRII cells of the CD8-TGFβRII Products comprise memory stem cells (T_{msc}) and/or central memory cells (Tₑₘ). In certain embodiments, at least about 35% of the CD8-TGFβRII cells of the CD8-TGFβRII Products comprise memory stem cells (T_{msc}) and/or central memory cells (T_{cm}). In certain embodiments, at least about 40% of the CD8-TGFβRII cells of the CD8-TGFβRII Products comprise memory stem cells (T_{msc}) and/or central memory cells (T_{cm}). In certain embodiments, at least about 45% of the CD8-TGFβRII cells of the CD8-TGFβRII Products comprise memory stem cells (T_{msc}) and/or central memory cells (T_{cm}). In certain embodiments, at least about 50% of the CD8-TGFβRII cells of the CD8-TGFβRII Products comprise memory stem cells (T_{msc}) and/or central memory cells (Tₑₘ). In certain embodiments, at least about 55% of the CD8-TGFβRII cells of the CD8-TGFβRII Products comprise memory stem cells (T_{msc}) and/or central memory cells (Tₑₘ). In certain embodiments, at least about 60% of the CD8-TGFβRII cells of the CD8-TGFβRII Products comprise memory stem cells (T_{msc}) and/or central memory cells (Tₑₘ). In certain embodiments, at least about 65% of the CD8-TGFβRII cells of the CD8-TGFβRII Products comprise memory stem cells (T_{msc}) and/or central memory cells (Tₑₘ). In certain embodiments, at least about 70% of the CD8-TGFβRII cells of the CD8-TGFβRII Products comprise memory stem cells (T_{msc}) and/or central memory cells (Tc T_{cm} m). In certain embodiments, at least about 75% of the CD8-TGFβRII cells of the CD8-TGFβRII Products comprise memory stem cells (T_{msc}) and/or central memory cells (T_{cm}). In certain embodiments, greater than about 75% of the CD8-TGFβRII cells of the CD8-TGFβRII Products comprise memory stem cells (T_{msc}) and/or central memory cells (Tₑₘ). In certain embodiments, T_{msc} can be CD45RA⁺CD62L⁺, CD28⁺CD95⁺, and CCR7⁺CD27⁺. In certain embodiments, T_{cm} can be CD45RO⁺CD62L⁺, CD28⁺CD95⁺, and CCR7⁺CD27⁺CD127⁺. Both Tₘₛ and T_{cm} are characterized as having weak effector T cell function, robust proliferation, robust engraftment, and long telomeres.

In certain embodiments, the CD8-TGFβRII Cells include a CD8 Construct. In certain embodiments, the CD8 Construct is knocked into the genome of the CD8-TGFβRII Cells. in certain embodiments, the CD8 Construct is selected from the group comprising CD8α homodimer (CD8 Construct 1), CD8α-P2A-CD8β (CD8 Construct 2), CD8α with CD8β intracellular domain (CD8 Construct 3), or CD8α homodimer with CD4 intracellular domain (CD8 Construct 4)

In certain embodiments, the CD8-TGFβRII Cells express CD8 Construct 1, CD8 Construct 2, CD8 Construct 3, or CD8 Construct 4. Specifically, using the gene editing technology and NeoTCR isolation technology described in PCT/US2020/17887 and PCT/US2019/025415, which are incorporated herein in their entireties, NeoTCRs are cloned in autologous CD8+ and CD4+ T cells from the same patient with cancer by precision genome engineered (using a DNA-mediated (non-viral) method as described in FIGS. 1A-IC) to express the NeoTCR.

Each of the CD8 Constructs, when expressed in a CD8-TGFβRII Cell, result in CD8-TGFβRII Cells and CD8-TGFβRII Cells Products. Table 2 provides a description of each construct and product.

TABLE 2
CD8 Constructs

| CD8 | | Exemplary |
|---|---|---|
| Construct | Expression components | constructs |
| CD8 | NeoTCR, CD8α extracellular domain, | FIG. 20A, |
| Construct 1 | CD8α transmembrane domain, CD8α | 21A, 22A |
| | intracellular domain | |
| CD8 | NeoTCR, CD8α extracellular domain, | 20B, 21B, 22A |
| Construct 2 | CD8α transmembrane domain, CD8α | |
| | intracellular domain, CD8β extracellular | |
| | domain, CD8β transmembrane domain, | |
| | CD8β intracellular domain | |
| CD8 | NeoTCR, CD8α extracellular domain, | 20C, 21C, 22B |
| Construct 3 | CD8α transmembrane domain, CD8β | |
| | intracellular domain | |
| CD8 | NeoTCR, CD8α extracellular domain, | 20D, 21D, 22B |
| Construct 4 | CD8α transmembrane domain, CD4 | |
| | intracellular domain | |

In certain embodiments, the CD8 constructs comprise a polynucleotide encoding a CD8 receptor. In certain embodiments, the CD8 receptor is a dimer. In certain embodiments, the CD8 receptor is a homodimer. In certain embodiments, the CD8 receptor is a heterodimer. In certain embodiments, the CD8 receptor includes a first monomer and a second monomer. In certain embodiments, the first monomer and the second monomer are the same. In certain embodiments, the first monomer and the second monomer are different. In certain embodiments, the first monomer of the CD8 receptor includes a polypeptide comprising a signal peptide, an extracellular domain, a transmembrane domain, and an intracellular domain. In certain embodiments, the second monomer of the CD8 receptor includes a polypeptide comprising a signal peptide, an extracellular domain, a transmembrane domain, and an intracellular domain.

In certain embodiments, the signal peptide comprises an amino acid sequence that is at least about 80%, about 85%, about 90%, about 95%, about 96%, about 97%, about 98%, or about 99% identical to the amino acid sequence set forth in SEQ ID NO: 9. In certain embodiments, the signal peptide comprises the amino acid sequence set forth in SEQ ID NO: 9. In certain embodiments, the signal peptide consists of the amino acid sequence set forth in SEQ ID NO: 9. SEQ ID NO: 9 is provided below.

[SEQ ID NO: 9]
MALPVTALLLPLALLLHAARP

In certain embodiments, the signal peptide comprises an amino acid sequence that is at least about 80%, about 85%, about 90%, about 95%, about 96%, about 97%, about 98%, or about 99% identical to the amino acid sequence set forth in SEQ ID NO: 13. In certain embodiments, the signal peptide comprises the amino acid sequence set forth in SEQ ID NO: 13. In certain embodiments, the signal peptide consists of the amino acid sequence set forth in SEQ ID NO: 13. SEQ ID NO: 13 is provided below.

[SEQ ID NO: 13]
MRPRLWLLLAAQLTVLHGNSV

In certain embodiments, the extracellular domain comprises an amino acid sequence that is at least about 80%, about 85%, about 90%, about 95%, about 96%, about 97%, about 98%, or about 99% identical to the amino acid sequence set forth in SEQ ID NO: 10. In certain embodiments, the extracellular domain comprises the amino acid sequence set forth in SEQ ID NO: 10. In certain embodiments, the extracellular domain consists of the amino acid sequence set forth in SEQ ID NO: 10. SEQ ID NO: 10 is provided below.

[SEQ ID NO: 10]
SQFRVSPLDRTWNLGETVELKCQVLLSNPTSGCSWLFQPRGAAASPTFLL
YLSONKPKAAEGLDTQRFSGKRLGDTFVLTLSDFRRENEGYYFCSALSNS
IMYFSHFVPVFLPAKPTTTPAPRPPTPAPTIASQPLSLRPEACRPAAGGA
VHTRGLDFACD

In certain embodiments, the extracellular domain comprises an amino acid sequence that is at least about 80%, about 85%, about 90%, about 95%, about 96%, about 97%, about 98%, or about 99% identical to the amino acid sequence set forth in SEQ ID NO: 14. In certain embodiments, the extracellular domain comprises the amino acid sequence set forth in SEQ ID NO: 14. In certain embodiments, the extracellular domain consists of the amino acid sequence set forth in SEQ ID NO: 14. SEQ ID NO: 14 is provided below.

[SEQ ID NO: 14]
LQQTPAYIKVQTNKMVMLSCEAKISLSNMRIYWLRQRQAPSSDSHHEFLA
LWDSAKGTIHGEEVEQEKIAVERDASRFILNLTSVKPEDSGIYFCMIVGS
PELTFGKGTQLSVVDFLPTTAQPTKKSTLKKRVCRLPRPETQKGPLCSP

In certain embodiments, the transmembrane domain comprises an amino acid sequence that is at least about 80%, about 85%, about 90%, about 95%, about 96%, about 97%, about 98%, or about 99% identical to the amino acid sequence set forth in SEQ ID NO: 11. In certain embodiments, the transmembrane domain comprises the amino acid sequence set forth in SEQ ID NO: 11. In certain embodiments, the transmembrane domain consists of the amino acid sequence set forth in SEQ ID NO: 11. SEQ ID NO: 11 is provided below.

[SEQ ID NO: 11]
IYIWAPLAGTCGVLLLSLVIT

In certain embodiments, the transmembrane domain comprises an amino acid sequence that is at least about 80%, about 85%, about 90%, about 95%, about 96%, about 97%, about 98%, or about 99% identical to the amino acid sequence set forth in SEQ ID NO: 15. In certain embodiments, the transmembrane domain comprises the amino acid sequence set forth in SEQ ID NO: 15. In certain embodiments, the transmembrane domain consists of the amino acid sequence set forth in SEQ ID NO: 15. SEQ ID NO: 15 is provided below.

[SEQ ID NO: 15]
ITLGLLVAGVLVLLVSLGVAI

In certain embodiments, the intracellular domain comprises an amino acid sequence that is at least about 80%, about 85%, about 90%, about 95%, about 96%, about 97%, about 98%, or about 99% identical to the amino acid sequence set forth in SEQ ID NO: 12. In certain embodiments, the intracellular domain comprises the amino acid sequence set forth in SEQ ID NO: 12. In certain embodiments, the intracellular domain consists of the amino acid sequence set forth in SEQ ID NO: 12. SEQ ID NO: 12 is provided below.

[SEQ ID NO: 12]
LYCNHRNRRRVCKCPRPVVKSGDKPSLSARYV

In certain embodiments, the intracellular domain comprises an amino acid sequence that is at least about 80%, about 85%, about 90%, about 95%, about 96%, about 97%, about 98%, or about 99% identical to the amino acid sequence set forth in SEQ ID NO: 16. In certain embodiments, the intracellular domain comprises the amino acid sequence set forth in SEQ ID NO: 16. In certain embodiments, the intracellular domain consists of the amino acid sequence set forth in SEQ ID NO: 16. SEQ ID NO: 16 is provided below.

[SEQ ID NO: 16]
HLCCRRRRARLRFMKQFYK

In certain embodiments, the intracellular domain comprises an amino acid sequence that is at least about 80%, about 85%, about 90%, about 95%, about 96%, about 97%, about 98%, or about 99% identical to the amino acid sequence set forth in SEQ ID NO: 17. In certain embodiments, the intracellular domain comprises the amino acid sequence set forth in SEQ ID NO: 17. In certain embodiments, the intracellular domain consists of the amino acid sequence set forth in SEQ ID NO: 17. SEQ ID NO: 17 is provided below.

[SEQ ID NO: 17]
CVRCRHRRRQAERMSQIKRLLSEKKTCQCPHRFQKTCSPI

In certain embodiments, the CD8 receptor (e.g., one expressed by a CD8 Construct) is a homodimer and comprises a first monomer and a second monomer. In certain embodiments, the first monomer and the second monomer comprise a signal peptide comprising an amino acid sequence that is at least about 80%, about 85%, about 90%, about 95%, about 96%, about 97%, about 98%, or about 99% identical to the amino acid sequence set forth in SEQ ID NO: 9, an extracellular domain comprising an amino acid sequence that is at least about 80%, about 85%, about 90%, about 95%, about 96%, about 97%, about 98%, or about 99% identical to the amino acid sequence set forth in SEQ ID NO: 10, a transmembrane domain comprising an amino acid sequence that is at least about 80%, about 85%, about 90%, about 95%, about 96%, about 97%, about 98%, or about 99% identical to the amino acid sequence set forth in SEQ ID NO: 11, and an intracellular domain comprising an amino acid sequence that is at least about 80%, about 85%, about 90%, about 95%, about 96%, about 97%, about 98%, or about 99% identical to the amino acid sequence set forth in SEQ ID NO: 12. In certain embodiments, the first monomer and the second monomer comprise a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12.

In certain embodiments, the first monomer and the second monomer comprise a signal peptide comprising an amino acid sequence that is at least about 80%, about 85%, about 90%, about 95%, about 96%, about 97%, about 98%, or about 99% identical to the amino acid sequence set forth in SEQ ID NO: 9, an extracellular domain comprising an amino acid sequence that is at least about 80%, about 85%, about 90%, about 95%, about 96%, about 97%, about 98%, or about 99% identical to the amino acid sequence set forth in SEQ ID NO: 10, a transmembrane domain comprising an amino acid sequence that is at least about 80%, about 85%, about 90%, about 95%, about 96%, about 97%, about 98%, or about 99% identical to the amino acid sequence set forth in SEQ ID NO: 11, and an intracellular domain comprising an amino acid sequence that is at least about 80%, about 85%, about 90%, about 95%, about 96%, about 97%, about 98%, or about 99% identical to the amino acid sequence set forth in SEQ ID NO: 16. In certain embodiments, the first monomer and the second monomer comprise a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 16.

In certain embodiments, the first monomer and the second monomer comprise a signal peptide comprising an amino acid sequence that is at least about 80%, about 85%, about 90%, about 95%, about 96%, about 97%, about 98%, or about 99% identical to the amino acid sequence set forth in SEQ ID NO: 9, an extracellular domain comprising an amino acid sequence that is at least about 80%, about 85%, about 90%, about 95%, about 96%, about 97%, about 98%, or about 99% identical to the amino acid sequence set forth in SEQ ID NO: 10, a transmembrane domain comprising an amino acid sequence that is at least about 80%, about 85%, about 90%, about 95%, about 96%, about 97%, about 98%, or about 99% identical to the amino acid sequence set forth in SEQ ID NO: 11, and an intracellular domain comprising an amino acid sequence that is at least about 80%, about 85%, about 90%, about 95%, about 96%, about 97%, about 98%, or about 99% identical to the amino acid sequence set forth in SEQ ID NO: 16. In certain embodiments, the first monomer and the second monomer comprise a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17.

In certain embodiments, the CD8 receptor (e.g., one expressed by a CD8 Construct) is a heterodimer and comprises a first monomer and a second monomer. In certain embodiments, the first monomer comprises a signal peptide comprising an amino acid sequence that is at least about 80%, about 85%, about 90%, about 95%, about 96%, about 97%, about 98%, or about 99% identical to the amino acid sequence set forth in SEQ ID NO: 9, an extracellular domain comprising an amino acid sequence that is at least about 80%, about 85%, about 90%, about 95%, about 96%, about 97%, about 98%, or about 99% identical to the amino acid sequence set forth in SEQ ID NO: 10, a transmembrane domain comprising an amino acid sequence that is at least about 80%, about 85%, about 90%, about 95%, about 96%, about 97%, about 98%, or about 99% identical to the amino acid sequence set forth in SEQ ID NO: 11, and an intracellular domain comprising an amino acid sequence that is at least about 80%, about 85%, about 90%, about 95%, about 96%, about 97%, about 98%, or about 99% identical to the amino acid sequence set forth in SEQ ID NO: 12; and the second monomer comprises a signal peptide comprising an amino acid sequence that is at least about 80%, about 85%, about 90%, about 95%, about 96%, about 97%, about 98%, or about 99% identical to the amino acid sequence set forth in SEQ ID NO: 13, an extracellular domain comprising an amino acid sequence that is at least about 80%, about 85%, about 90%, about 95%, about 96%, about 97%, about 98%, or about 99% identical to the amino acid sequence set forth in SEQ ID NO: 14, a transmembrane domain comprising an amino acid sequence that is at least about 80%, about 85%, about 90%, about 95%, about 96%, about 97%, about 98%, or about 99% identical to the amino acid sequence set forth in SEQ ID NO: 15, and an intracellular domain comprising an amino acid sequence that is at least about 80%, about 85%, about 90%, about 95%, about 96%, about 97%, about 98%, or about 99% identical to the amino acid sequence set forth in SEQ ID NO: 16. In certain embodiments, the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12; and the second monomer comprises a signal peptide comprising the amino acid sequence set forth in SEQ ID NO: 13, an extracellular domain comprising the amino acid sequence set forth in SEQ ID NO: 14, a transmembrane domain comprising the amino acid sequence set forth in SEQ ID NO: 15, and an intracellular domain comprising the amino acid sequence set forth in SEQ ID NO: 16.

In certain embodiments, the CD8-TGFβRII Cells that express CD8 Construct 1 comprise a NeoTCR and a CD8 homodimer. In certain embodiments, the CD8-TGFβRII Cells that express CD8 Construct 1 comprise the expression of a NeoTCR, a CD8α extracellular domain, a CD8α transmembrane domain, and a CD8α intracellular domain. In certain embodiments, the CD8-TGFβRII Cells that express CD8 Construct 1 further include the expression of a CD8α signal peptide. In certain embodiments, the CD8-TGFβRII Cells that express CD8 Construct 1 comprise the translated elements presented in FIG. 20A. In certain embodiments, the CD8-TGFβRII Cells that express CD8 Construct 1 comprise a NeoTCR, a CD8α signal peptide (SEQ ID NO:9), a CD8α extracellular domain (SEQ ID NO: 10), a CD8α transmembrane domain (SEQ ID NO: 11), and a CD8α intracellular domain (SEQ ID NO: 12). In certain embodiments, sequence modifications of the CD8α signal peptide, CD8α extracellular domain, CD8α transmembrane domain, and CD8α intracellular domain can be made that conserve or substantially conserve the function of each element. In certain embodiments, such sequence modifications are conservative substitutions of amino acids.

In certain embodiments, the sequence of CD8 Construct 1 can be modified in any number of ways so long as the translation of the CD8α signal peptide, CD8α extracellular domain, CD8α transmembrane domain, and CD8α intracellular domain leave each element with conserved function. In certain embodiments, the order of each element of the CD8 Construct 1 remains the same but the sequences of each individual element can be changed so long as the amino acids that the nucleic acid encodes remain the same or only comprise conservative substitutions. In certain embodiments, the order of each element of the CD8 Construct 1 remains the same but the sequences of each individual element can be changed so long as the function of the encoded proteins remains substantially unchanged.

In certain embodiments, any of the CD8-TGFβRII Cells that express CD8 Construct 1 are manufactured to make a CD8-TGFβRII Product.

In certain embodiments, the CD8-TGFβRII Cells that express CD8 Construct 2 comprise a NeoTCR, a CD8α, and aCD8β. In certain embodiments, the CD8α and CD8β are separated by a protease cleavage site and a 2A peptide in the CD8 Construct 2 of the CD8-TGFβRII Cells. In certain embodiments, the CD8-TGFβRII Cells that express CD8 Construct 2 comprise the expression of a NeoTCR, a CD8α extracellular domain, a CD8α transmembrane domain, a CD8α intracellular domain, a CD8β extracellular domain, a CD8β transmembrane domain, and a CD8β intracellular domain. In certain embodiments, the CD8-TGFβRII Cells that express CD8 Construct 2 further comprise the expression of a CD8α signal peptide. In certain embodiments, the CD8-TGFβRII Cells that express CD8 Construct 2 further comprise the expression of a CD8β signal peptide. In certain embodiments, the CD8-TGFβRII Cells that express CD8 Construct 2 further comprise the expression of a CD8α signal peptide and a CD8β signal peptide. In certain embodiments, the CD8-TGFβRII Cells that express CD8 Construct 2 comprise the translated elements presented in FIG. 20B. In a non-limiting exemplary embodiment, the CD8-TGFβRII Cells that express CD8 Construct 2 comprise a NeoTCR, a CD8α signal peptide (SEQ ID NO: 9), a CD8α extracellular domain (SEQ ID NO: 10), a CD8α transmembrane domain (SEQ ID NO: 11), a CD8α intracellular domain (SEQ ID NO: 12), a CD8β signal peptide (SEQ ID NO: 13), a CD8β extracellular domain (SEQ ID NO:14), a CD8β transmembrane domain (SEQ ID NO:15), and a CD8β intracellular domain (SEQ ID NO: 16). In certain embodiments, sequence modifications of the CD8α signal peptide, CD8α extracellular domain, CD8α transmembrane domain, CD8α intracellular domain, CD8β extracellular domain, CD8β transmembrane domain, and CD8β intracellular domain can be made that conserve or substantially conserve the function of each element. In certain embodiments, such sequence modifications are conservative substitutions of amino acids.

In certain embodiments, the sequence of CD8 Construct 2 can be modified in any number of ways so long as the translation of the CD8α signal peptide, CD8α extracellular domain, CD8α transmembrane domain, CD8α intracellular domain, CD8β extracellular domain, CD8β transmembrane domain, and CD8β intracellular domain leave each element with conserved function. In certain embodiments, the order of each element of the CD8 Construct 2 remains the same but the sequences of each individual element can be changed so long as the amino acids that the nucleic acid encodes remain the same or only comprise conservative substitutions. In certain embodiments, the order of each element of the CD8 Construct 2 remains the same but the sequences of each individual element can be changed so long as the function of the encoded proteins remains substantially unchanged.

In certain embodiments, any of the CD8-TGFβRII Cells that express CD8 Construct 2 are manufactured to make a CD8-TGFβRII Product.

In certain embodiments, the CD8-TGFβRII Cells that express CD8 Construct 3 comprise a NeoTCR and a CD8α with CD8β intracellular domain. In certain embodiments, the CD8-TGFβRII Cells that express CD8 Construct 3 comprise the expression of a NeoTCR, a CD8α extracellular domain, a CD8α transmembrane domain, and a CD8β intracellular domain. In certain embodiments, the CD8-TGFβRII Cells that express CD8 Construct 3 further include the expression of a CD8α signal peptide. In certain embodiments, the CD8 Product 3 comprises the translated elements presented in FIG. 20C. In certain embodiments, the CD8-TGFβRII Cells that express CD8 Construct 3 comprise a NeoTCR, a CD8α signal peptide (SEQ ID NO:9), a CD8α extracellular domain (SEQ ID NO: 10), a CD8α transmembrane domain (SEQ ID NO: 11), and a CD8β intracellular domain (SEQ ID NO: 16). In certain embodiments, sequence modifications of the CD8α signal peptide, CD8α extracellular domain, CD8α transmembrane domain, and CD8β intracellular domain can be made that conserve or substantially conserve the function of each element. In certain embodiments, such sequence modifications are conservative substitutions of amino acids.

In certain embodiments, the sequence of the CD8 Construct 3 can be modified in any number of ways so long as the translation of the CD8α signal peptide, CD8α extracellular domain, CD8α transmembrane domain, and CD8β intracellular domain leave each element with conserved function. In certain embodiments, the order of each element of the CD8 Construct 3 remains the same but the sequences of each individual element can be changed so long as the amino acids that the nucleic acid encodes remain the same or only comprise conservative substitutions. In certain embodiments, the order of each element of the CD8 Construct 3 remains the same but the sequences of each individual element can be changed so long as the function of the encoded proteins remain substantially unchanged.

In certain embodiments, any of the CD8-TGFβRII Cells that express CD8 Construct 3 are manufactured to make a CD8-TGFβRII Product.

In certain embodiments, the CD8-TGFβRII Cells that express CD8 Construct 4 comprises a NeoTCR and a CD8α homodimer with CD4 intracellular domain. In certain embodiments, the CD8-TGFβRII Cells that express CD8 Construct 4 comprise the expression of a NeoTCR, a CD8α extracellular domain, aCD8α transmembrane domain, and a CD4 intracellular domain. In certain embodiments, the CD8-TGFβRII Cells that express CD8 Construct 4 further include the expression of a CD8α signal peptide. In certain embodiments, the CD8-TGFβRII Cells that express CD8 Construct 4 comprise the translated elements presented in FIG. 20D. In certain embodiments, the CD8-TGFβRII Cells that express CD8 Construct 4 comprises a NeoTCR, a CD8α signal peptide (SEQ ID NO:9), a CD8α extracellular domain (SEQ ID NO: 10), a CD8α transmembrane domain (SEQ ID NO: 11), and a CD4 intracellular domain (SEQ ID NO: 17). In certain embodiments, sequence modifications of the CD8α signal peptide, CD8α extracellular domain, CD8α transmembrane domain, and CD4 intracellular domain can be made that conserve or substantially conserve function of each element. In certain embodiments, such sequence modifications are conservative substitutions of amino acids.

In certain embodiments, the sequence of CD8 Construct 4 can be modified in any number of ways so long as the translation of the CD8α signal peptide, CD8α extracellular domain, CD8α transmembrane domain, and CD4 intracellular domain leave each element with conserved function. In certain embodiments, the order of each element of the CD8 Construct 4 remains the same but the sequences of each individual element can be changed so long as the amino acids that the nucleic acid encodes remain the same or only comprise conservative substitutions. In certain embodiments, the order of each element of the CD8 Construct 4 remains the same but the sequences of each individual element can be changed so long as the function of the encoded proteins remains substantially unchanged.

In certain embodiments, any of the CD8-TGFβRII Cells that express CD8 Construct 4 are manufactured to make a CD8-TGFβRII Product.

2.4. Methods of Producing Products with a Young Phenotype

In certain embodiments, the present disclosure relates, in part, to the production of engineered “young” T cells. In certain embodiments, the NeoTCR Cells are “young” T cells. In certain embodiments, the TGFβRII Cells are “young” T cells. In certain embodiments, the dnTGFβRII Cells are “young” T cells. In certain embodiments, the CD8-TGFβRII Cells are “young” T cells. In certain embodiments, the present disclosure comprises methods for producing antigen-specific cells, e.g., T cells, ex vivo, comprising activating, engineering, and expanding antigen-specific cells originally obtained from a subject or isolated from such sample.

In certain embodiments, the methods for activating cells comprise the steps of activating the TCR/CD3 complex. For example, without limitation, the T cells can be incubated and/or cultured with CD3 agonists, CD28 agonists, or a combination thereof.

In certain embodiments, engineered activated antigen-specific cells, e.g., engineered activated T cells, can be expanded by culturing the engineered activated antigen-specific cells, e.g., T cells, with cytokines, chemokine, soluble peptides, or combination thereof. In certain embodiments, the engineered activated antigen-specific cells, e.g., engineered activated T cells, can be cultured with one or more cytokines. In certain embodiments, the cytokines can be IL2, IL7, IL15, or combinations thereof. For example, engineered activated antigen-specific cells, e.g., engineered activated T cells, can be cultured with IL7 and IL15. In certain embodiments, the cytokine used in connection with the engineered activated antigen-specific cell, e.g., engineered activated T cell, culture can be present at a concentration from about 1 pg/ml to about 1 g/ml, from about 1 ng/ml to about 1 g/ml, from about 1 μg/ml to about 1 g/ml, or from about 1 mg/ml to about 1 g/ml, and any values in between.

### 2.5. Gene-Editing Methods

In certain embodiments, the present disclosure involves, in part, methods of engineering human cells, e.g., engineered T cells or engineered human stem cells. In certain embodiments, the present disclosure involves, in part, methods of engineering human cells, e.g., NK cells, NKT cells, macrophages, hematopoietic stem cells (HSCs), cells derived from HSCs, or dendritic/antigen-presenting cells. In certain embodiments, such engineering involves genome editing. For example, but not by way of limitation, such genome editing can be accomplished with nucleases targeting one or more endogenous loci, e.g., TCR alpha (TCRa) locus, TCR beta (TCRβ) locus, and TGFβRII locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, a CRISPR/Cas nuclease system is used to engineer human cells. In certain embodiments, the CRISPR/Cas nuclease system comprises a Cas nuclease and one or more RNAs that recruit the Cas nuclease to the endogenous target sequence, e.g., single guide RNA. In certain embodiments, the Cas nuclease and the RNA are introduced in the cell separately, e.g. using different vectors or compositions, or together, e.g., in a polycistronic construct or a single protein-RNA complex. In certain embodiments, the Cas nuclease is Cas9 or Cas12a. In certain embodiments, the Cas9 polypeptide is obtained from a bacterial species including, without limitation, Streptococcus pyogenes or Neisseria menengitidis. Additional examples of CRISPR/Cas systems are known in the art. See Adli, Mazhar. “The CRISPR tool kit for genome editing and beyond.” Nature communications vol. 9, 1 1911 (2018), herein incorporated by reference for all that it teaches.

In certain embodiments, genome editing occurs at one or more genome loci that regulate immunological responses. In certain embodiments, the loci include, without limitation, TCR alpha (TCRα) locus, TCR beta (TCRβ) locus, TCR gamma (TCRγ), and TCR delta (TCRδ). In certain embodiments, the loci include a TGFβRII locus. In certain embodiments, the locus for inserting a CD8 Construct is anywhere in the genome. In certain embodiments, the locus for inserting a CD8 Construct is the TRAC locus. In certain embodiments, the locus for inserting a CD8 Construct is one of the two TRBC loci. In certain embodiments, the locus for inserting a CD8 Construct is a locus other than the TRAC locus or TRAB loci. In certain embodiments, the loci for inserting a CD8 Construct is inserted into a gene locus wherein such gene is knocked out. By way of a non-limiting example, if the desired phenotype of a CD8 Product is the expression of a NeoTCR, the expression of a CD8 Construct, and the knockout of the TET2 gene or the AAVS1 gene, the CD8 Construct can be inserted at the TET2 locus or AAVS1 locus. In another non-limiting example, if the desired phenotype of a CD8 Product is the expression of a NeoTCR, the expression of a CD8 Construct, and the knockout of the TGFβRII gene, the CD8 Construct can be inserted at the TGFβRII locus. In certain embodiments, the insertion of the CD8 Construct is in tandem with the NeoTCR insertion. In certain embodiments, the insertion of the CD8 Construct is a separate locus from the NeoTCR insertion.

In certain embodiments, the locus for inserting a dnTGFβRII Construct is the TRAC locus. In certain embodiments, the locus for inserting a dnTGFβRII Construct is one of the two TRBC loci. In certain embodiments, the locus for inserting a dnTGFβRII Construct is a locus other than the TRAC locus or TRAB loci. In certain embodiments, the loci for inserting a dnTGFβRII Construct is inserted into a gene locus wherein such gene is knocked out. By way of a non-limiting example, if the desired phenotype of a dnTGFβRII Product is the expression of a NeoTCR, the expression of a dnTGFβRII Construct, and the knockout of the TET2 gene or the AAVS1 gene, the dnTGFβRII Construct can be inserted at the TET2 locus or AAVS1 locus. In another non-limiting example, if the desired phenotype of a dnTGFβRII Product is the expression of a NeoTCR, the expression of a dnTGFβRII Construct, and the knockout of the TGFβRII gene, the dnTGFβRII Construct can be inserted at the TGFβRII locus. In certain embodiments, the insertion of the dnTGFβRII Construct is in tandem with the NeoTCR insertion. In certain embodiments, the insertion of the dnTGFβRII Construct is a separate locus from the NeoTCR insertion.

In certain embodiments, genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for the delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, genome editing is performed by using viral delivery systems. In certain embodiments, the viral methods include targeted integration (including but not limited to AAV) and random integration (including but not limited to lentiviral approaches). In certain embodiments, the viral delivery would be accomplished without integration of the nuclease. In such embodiments, the viral delivery system can be Lentiflash or another similar delivery system.

### 2.6. Homology Recombination Templates

In certain embodiments, the present disclosure provides genome editing of a cell by introducing and recombining homologous recombination (HR) template nucleic acid sequence into an endogenous locus of a cell. In certain embodiments, the HR template nucleic acid sequence is linear. In certain embodiments, the HR template nucleic acid sequence is circular. In certain embodiments, the circular HR template can be a plasmid, minicircle, or nanoplasmid. In certain embodiments, the HR template nucleic acid sequence comprises first and second homology arms. In certain embodiments, the homology arms can be of about 300 bases to about 2,000 bases. For example, each homology arm can be 1,000 bases. In certain embodiments, the homology arms can be homologous to first and second endogenous sequences of the cell. In certain embodiments, the endogenous locus is a TCR locus. For example, the first and second endogenous sequences are within a TCR alpha locus or a TCR beta locus.

In certain embodiments, the first homology arm comprises a nucleotide sequence that is at least about 80%, about 85%, about 90%, about 95%, about 96%, about 97%, about 98%, or about 99% identical to the nucleotide sequence set forth in SEQ ID NO: 29. In certain embodiments, the first homology arm comprises the nucleotide sequence set forth in SEQ ID NO: 29. In certain embodiments, the first homology arm consists of the nucleotide sequence set forth in SEQ ID NO: 29. In certain embodiments, the first homology arm comprises about 300 consecutive bases, about 400 consecutive bases, about 500 consecutive bases, about 600 consecutive bases, about 700 consecutive bases, about 800 consecutive bases, or about 900 consecutive bases of SEQ ID NO: 29. In certain embodiments, the first homology arm comprises about 300 consecutive bases of SEQ ID NO: 29. In certain embodiments, the first homology arm comprises about 400 consecutive bases of SEQ ID NO: 29. In certain embodiments, the first homology arm comprises about 600 consecutive bases of SEQ ID NO: 29.

In certain embodiments, the second homology arm comprises a nucleotide sequence that is at least about 80%, about 85%, about 90%, about 95%, about 96%, about 97%, about 98%, or about 99% identical to the nucleotide sequence set forth in SEQ ID NO: 30. In certain embodiments, the second homology arm comprises the nucleotide sequence set forth in SEQ ID NO: 30. In certain embodiments, the second homology arm consists of the nucleotide sequence set forth in SEQ ID NO: 30. In certain embodiments, the second homology arm comprises about 300 consecutive bases, about 400 consecutive bases, about 500 consecutive bases, about 600 consecutive bases, about 700 consecutive bases, about 800 consecutive bases, or about 900 consecutive bases of SEQ ID NO: 30. In certain embodiments, the second homology arm comprises about 300 consecutive bases of SEQ ID NO: 30. In certain embodiments, the second homology arm comprises about 400 consecutive bases of SEQ ID NO: 30. In certain embodiments, the second homology arm comprises about 600 consecutive bases of SEQ ID NO: 30.

SEQ ID NO: 29 and SEQ ID NO: 30 are provided below.

SEQ ID NO: 29]
ACATTAAAAACACAAAATCCTACGGAAATACTGAAGAATGAGTCTCAGCA
CTAAGGAAAAGCCTCCAGCAGCTCCTGCTTTCTGAGGGTGAAGGATAGAC
GCTGTGGCTCTGCATGACTCACTAGCACTCTATCACGGCCATATTCTGGC
AGGGTCAGTGGCTCCAACTAACATTTGTTTGGTACTTTACAGTTTATTAA
ATAGATGTTTATATGGAGAAGCTCTCATTTCTTTCTCAGAAGAGCCTGGC
TAGGAAGGTGGATGAGGCACCATATTCATTTTGCAGGTGAAATTCCTGAG
ATGTAAGGAGCTGCTGTGACTTGCTCAAGGCCTTATATCGAGTAAACGGT
AGTGCTGGGGCTTAGACGCAGGTGTTCTGATTTATAGTTCAAAACCTCTA
TCAATGAGAGAGCAATCTCCTGGTAATGTGATAGATTTCCCAACTTAATG
CCAACATACCATAAACCTCCCATTCTGCTAATGCCCAGCCTAAGTTGGGG
AGACCACTCCAGATTCCAAGATGTACAGTTTGCTTTGCTGGGCCTTTTTC
CCATGCCTGCCTTTACTCTGCCAGAGTTATATTGCTGGGGTTTTGAAGAA
GATCCTATTAAATAAAAGAATAAGCAGTATTATTAAGTAGCCCTGCATTT
CAGGTTTCCTTGAGTGGCAGGCCAGGCCTGGCCGTGAACGTTCACTGAAA
TCATGGCCTCTTGGCCAAGATTGATAGCTTGTGCCTGTCCCTGAGTCCCA
GTCCATCACGAGCAGCTGGTTTCTAAGATGCTATTTCCCGTATAAAGCAT
GAGACCGTGACTTGCCAGCCCCACAGAGCCCCGCCCTTGTCCATCACTGG
CATCTGGACTCCAGCCTGGGTTGGGGCAAAGAGGGAAATGAGATCATGTC
CTAACCCTGATCCTCTTGTCCCACAGATATCCAGAACCCTGACCCTGCCG
TGTACCAGCTGAGAGACTCTAAATCCAGTGACAAGTCTGTCTGCCTATTC
[SEQ ID NO: 30]
ACCGATTTTGATTCTCAAACAAATGTGTCACAAAGTAAGGATTCTGATGT
GTATATCACAGACAAAACTGTGCTAGACATGAGGTCTATGGACTTCAAGA
GCAACAGTGCTGTGGCCTGGAGCAACAAATCTGACTTTGCATGTGCAAAC
GCCTTCAACAACAGCATTATTCCAGAAGACACCTTCTTCCCCAGCCCAGG
TAAGGGCAGCTTTGGTGCCTTCGCAGGCTGTTTCCTTGCTTCAGGAATGG
CCAGGTTCTGCCCAGAGCTCTGGTCAATGATGTCTAAAACTCCTCTGATT
GGTGGTCTCGGCCTTATCCATTGCCACCAAAACCCTCTTTTTACTAAGAA
ACAGTGAGCCTTGTTCTGGCAGTCCAGAGAATGACACGGGAAAAAAGCAG
ATGAAGAGAAGGTGGCAGGAGAGGGCACGTGGCCCAGCCTCAGTCTCTCC
AACTGAGTTCCTGCCTGCCTGCCTTTGCTCAGACTGTTTGCCCCTTACTG
CTCTTCTAGGCCTCATTCTAAGCCCCTTCTCCAAGTTGCCTCTCCTTATT
TCTCCCTGTCTGCCAAAAAATCTTTCCCAGCTCACTAAGTCAGTCTCACG
CAGTCACTCATTAACCCACCAATCACTGATTGTGCCGGCACATGAATGCA
CCAGGTGTTGAAGTGGAGGAATTAAAAAGTCAGATGAGGGGTGTGCCCAG
AGGAAGCACCATTCTAGTTGGGGGAGCCCATCTGTCAGCTGGGAAAAGTC
CAAATAACTTCAGATTGGAATGTGTTTTAACTCAGGGTTGAGAAAACAGC
TACCTTCAGGACAAAAGTCAGGGAAGGGCTCTCTGAAGAAATGCTACTTG
AAGATACCAGCCCTACCAAGGGCAGGGAGAGGACCCTATAGAGGCCTGGG
ACAGGAGCTCAATGAGAAAGGAGAAGAGCAGCAGGCATGAGTTGAATGAA
GGAGGCAGGGCCGGGTCACAGGGCCTTCTAGGCCATGAGAGGGTAGACAG

In certain embodiments, the HR template comprises a TCR gene sequence. In non-limiting embodiments, the TCR gene sequence is a patient specific TCR gene sequence. In non-limiting embodiments, the TCR gene sequence is tumor-specific. In non-limiting embodiments, the TCR gene sequence can be identified and obtained using the methods described in PCT/US2020/017887, the content of which is herein incorporated by reference. In certain embodiments, the HR template comprises a TCR alpha gene sequence and a TCR beta gene sequence.

In certain embodiments, the HR template is a polycistronic polynucleotide. In certain embodiments, the HR template comprises sequences encoding for flexible polypeptide sequences (e.g., Gly-Ser-Gly sequence). In certain embodiments, the HR template comprises sequences encoding an internal ribosome entry site (IRES). In certain embodiments, the HR template comprises a 2A peptide (e.g., P2A, T2A, E2A, and F2A). In certain embodiments, the HR template comprises a protease cleavage site. In certain embodiments, the HR template comprises a signal sequence.

In certain embodiments, the flexible polypeptide encodes a glycine-serine-glycine sequence. In certain embodiments, the flexible polypeptide is encoded by a nucleotide sequence comprising the nucleotide sequence set forth in SEQ ID NO: 31 or SEQ ID NO: 32. In certain embodiments, the flexible polypeptide is encoded by a nucleotide sequence consisting of the nucleotide sequence set forth in SEQ ID NO: 31 or SEQ ID NO: 32. SEQ ID NO: 31 and SEQ ID NO: 32 are provided below.

[SEQ ID NO: 31]
GGCAGCGGC
[SEQ ID NO: 32]
GGCTCCGGA

In certain embodiments, the 2A peptide is a P2A peptide. In certain embodiments, the P2A peptide comprises the amino acid sequence set forth in SEQ ID NO: 18. In certain embodiments, the P2A peptide consists of the amino acid sequence set forth in SEQ ID NO: 18. In certain embodiments, the P2A peptide is encoded by a nucleotide sequence comprising the nucleotide sequence set forth in SEQ ID NO: 33 or SEQ ID NO: 34. In certain embodiments, the P2A peptide is encoded by a nucleotide sequence consisting of the nucleotide sequence set forth in SEQ ID NO: 33 or SEQ ID NO: 34. SEQ ID NOs: 33 and 34 are provided below.

[SEQ ID NO: 33]
GCCACCAACTTCAGCCTGCTGAAGCAGGCCGGCGACGTGGAGGAGAACCC
CGGCCCT
[SEQ ID NO: 34]
GCCACTAACTTCTCCCTGTTGAAACAGGCTGGCGATGTTGAAGAAAACCC
CGGTCCT

In certain embodiments, the P2A peptide comprises, at its N-end, a flexible polypeptide comprising a glycine-serine-glycine sequence. In certain embodiments, the P2A peptide comprises, at its N-end, a flexible polypeptide consisting of a glycine-serine-glycine sequence. In certain embodiments, the P2A peptide comprises the amino acid sequence set forth in SEQ ID NO: 19. In certain embodiments, the P2A peptide consists of the amino acid sequence set forth in SEQ ID NO: 19. In certain embodiments, the protease cleavage sites within any of the constructs described herein (e.g., CD8 Constructs or NeoTCR Constructs) are furin protease cleavage sites. In certain embodiments, the sequence of the furin protease cleavage site comprises any one of the sequences provided in Table 3.

TABLE 3

Furin Sequences

| Furin sequence | SEQ ID NO |
|---|---|
| RRRR | SEQ ID NO: 4 |
| RRKR | SEQ ID NO: 5 |
| RKRR | SEQ ID NO: 6 |
| RKKR | SEQ ID NO: 7 |

In certain embodiments, the signal sequence is a human growth hormone (HGH) signal sequence. In certain embodiments, the HGH signal sequence comprises the amino acid sequence set forth in SEQ ID NO: 35. In certain embodiments, the HGH signal sequence consists of the amino acid sequence set forth in SEQ ID NO: 35. In certain embodiments, the HGH signal sequence is encoded by a nucleotide sequence comprising the nucleotide sequence set forth in SEQ ID NO: 36 or SEQ ID NO: 37. In certain embodiments, the HGH signal sequence is encoded by a nucleotide sequence consisting of the nucleotide sequence set forth in SEQ ID NO: 36 or SEQ ID NO: 37. SEQ ID NOs: 35-37 are provided below.

[SEQ ID NO: 35]
MATGSRTSLLLAFGLLCLPWLQEGSA
[SEQ ID NO: 36]
ATGGCCACAGGCAGCAGAACATCTCTGCTGCTGGCCTTCGGACTGCTGTG
TCTGCCTTGGCTGCAAGAGGGTTCCGCC
[SEQ ID NO: 37]
ATGGCCACCGGCTCTAGAACAAGCCTGCTGCTCGCTTTTGGCCTGCTCTG
CCTCCCATGGCTCCAAGAAGGATCTGCT

In certain embodiments, the HR template includes multiple 2A peptides, flexible polypeptides, protease cleavage peptides, signal peptides, or combinations thereof. In certain embodiments, the multiple 2A peptides can have the same amino acid sequence. In certain embodiments, the multiple flexible polypeptides can have the same amino sequence. In certain embodiments, the multiple protease cleavage peptides can have the same amino acid sequence. In certain embodiments, the multiple signal peptides can have the same amino acid sequence. In certain embodiments, when the HR template includes multiple 2A peptides, flexible polypeptides, protease cleavage peptides, signal peptides, or combinations thereof, these amino acid sequences are encoded by codon diverged nucleotide sequences.

Additional information on the HR template nucleic acids and methods of modifying a cell thereof can be found in International Patent Application no. PCT/US2018/058230, the content of which is herein incorporated by reference.

2.6.1. Exemplified Homology Recombination Templates

In certain embodiments, the HR template comprises a sequence encoding an exogenous TCR and a sequence encoding a CD8 receptor. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template comprises, from 5′ to 3′, a first homology arm, a first 2A peptide, a first signal sequence peptide, a sequence encoding a CD8 receptor, a protease cleavage site, a second 2A peptide, a second signal sequence, a TCRβ gene sequence, a second protease cleavage site, a third 2A peptide, a third signal sequence peptide, and a TRCα gene sequence.

In certain embodiments, the HR template comprises a sequence encoding an exogenous TCR and a dnTGFβRII. In certain embodiments, the sequence encoding an exogenous TCR encodes a TRCα and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a poly-A signal sequence. In certain embodiments, the HR template further comprises a promoter and an insulator. In certain embodiments, the HR template further comprises a first homology arm and a second homology arm. In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a dnTGFβRII, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a first poly-A signal sequence and a second poly-A signal sequence. In certain embodiments, the HR template further comprises a promoter, an insulator, and a WPRE element. In certain embodiments, the HR template further comprises a first homology arm and a second homology arm. In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, an insulator, a promoter, a dnTGFβRII, a WPRE element, a second poly-A signal sequence, and a second homology arm.

In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, a first insulator, a promoter, a dnTGFβRII, a second poly-A signal sequence, a second insulator, and a second homology arm.

In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a first poly-A signal sequence, an insulator, a promoter, a dnTGFβRII, a second poly-A signal sequence, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a poly-A signal sequence. In certain embodiments, the HR template further comprises a promoter and a WPRE element. In certain embodiments, the HR template further comprises a first homology arm and a second homology arm. In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a WPRE element, a dnTGFβRII, a promoter, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template comprises, from 5′ to 3′, a first homology arm, a first 2A peptide, a first signal sequence peptide, a dnTGFβRII, a protease cleavage site, a second 2A peptide, a second signal sequence, a TCRβ gene sequence, a second protease cleavage site, a third 2A peptide, a third signal sequence peptide, and a TRCα gene sequence.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a poly-A signal sequence and an insulator. In certain embodiments, the 3′ of the dnTGFβRII comprises a STOP codon. In certain embodiments, the HR template further comprises a promoter. In certain embodiments, the HR template further comprises a first homology arm and a second homology arm. In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a dnTGFβRII having at its 3′ a termination codon, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a poly-A signal sequence. In certain embodiments, the 3′ of the dnTGFβRII comprises a protease cleavage site and a 2A peptide. In certain embodiments, the HR template further comprises a promoter. In certain embodiments, the HR template further comprises a first homology arm and a second homology arm. In certain embodiments, the HR template comprises, from 5′ to 3′, a first homology arm, a first 2A peptide, a first signal sequence peptide, a TCRβ gene sequence, a protease cleavage site, a second 2A peptide, a second signal sequence peptide, a TRCα gene sequence, a poly-A signal sequence, a promoter, a dnTGFβRII, a second protease cleavage site, a third 2A peptide, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template comprises an enhancer. In certain embodiments, the HR template further comprises a poly-A signal sequence and a pause element. In certain embodiments, the 3′ of the dnTGFβRII comprises protease cleavage site and a 2A peptide. In certain embodiments, the HR template further comprises a promoter. In certain embodiments, the HR template further comprises a first homology arm and a second homology arm. In certain embodiments, the HR template comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, a pause element, a promoter, a dnTGFβRII, a second protease cleavage site, a third 2A peptide, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template comprises an enhancer. In certain embodiments, the HR template further comprises a poly-A signal sequence and an insulator. In certain embodiments, the 3′ of the dnTGFβRII comprises a protease cleavage site and a 2A peptide. In certain embodiments, the HR template further comprises a promoter. In certain embodiments, the HR template further comprises a first homology arm and a second homology arm. In certain embodiments, the HR template comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a dnTGFβRII, a second protease cleavage site, a third 2A peptide, and a second homology arm.

In certain embodiments, the HR template comprises a sequence encoding an exogenous TCR and a dnTGFβRII. In certain embodiments, the sequence encoding an exogenous TCR encodes a TRCα gene sequence and a TCRβ gene sequence. In certain embodiments, the sequence encoding an exogenous TCR comprises a first 2A peptide and a second 2A peptide, a first signal peptide and a second signal peptide, and a protease cleavage site. In certain embodiments, the HR template further comprises a poly-A signal sequence, an enhancer, and an insulator. In certain embodiments, the 3′ of the dnTGFβRII comprises a STOP codon. In certain embodiments, the HR template further comprises a promoter. In certain embodiments, the HR template further comprises a first homology arm and a second homology arm. In certain embodiments, the HR template comprises, from 5′ to 3′, a first homology arm, an enhancer, a first 2A peptide, a first signal sequence peptide, a TCRβ gene sequence, a protease cleavage site, a second 2A peptide, a second signal sequence peptide, a TRCα gene sequence, a poly-A signal sequence, an insulator, a promoter, a dnTGFβRII having at its 3′ a termination codon, and a second homology arm.

### 2.7. Compositions and Vectors

The presently disclosed subject matter provides compositions comprising cells (e.g., NeoTCR cells or CD8-TGFβRII Cells) disclosed herein.

In certain embodiments, the presently disclosed subject matter provides nucleic acid compositions comprising a polynucleotide encoding the NeoTCR disclosed herein. In certain embodiments, the nucleic acid compositions disclosed herein comprise a polynucleotide encoding a CD8 Construct or a dnTGFβRII Construct disclosed herein. Also provided are cells comprising such nucleic acid compositions.

In certain embodiments, the nucleic acid composition further comprises a promoter that is operably linked to the NeoTCR disclosed herein. In certain embodiments, the nucleic acid composition further comprises a promoter that is operably linked to the exogenous CD8 of the CD8 Construct or the dnTGFβRII of the dnTGFβRII Construct disclosed herein.

In certain embodiments, the promoter is endogenous or exogenous. In certain embodiments, the exogenous promoter is selected from the group consisting of an elongation factor (EF)-1 promoter, a CMV promoter, an MND promoter, a SV40 promoter, a PGK promoter, a long terminal repeat (LTR) promoter and a metallothionein promoter. In certain embodiments, the promoter is an inducible promoter. In certain embodiments, the inducible promoter is selected from the group consisting of an NFAT transcriptional response element (TRE) promoter, a CD69 promoter, a CD25 promoter, an IL-2 promoter, an IL-12 promoter, a p40 promoter, and a Bcl-xL promoter.

The compositions and nucleic acid compositions can be administered to subjects or and/delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. Non-viral vectors may be used as well.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for the delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### 2.8. Cells

The presently disclosed subject matter provides cells genetic modifications disclosed herein. In certain embodiments, the cell is a NeoTCR cell. In certain embodiments, the cell is a CD8 Cell. In certain embodiments, the cell is a TGFβRII cell. In certain embodiments, the cell is a dnTGFβRII cell. In certain embodiments, the cell is a CD8-TGFβRII cell.

In certain embodiments, the cell is an immune cell (e.g., a lymphocyte). For example, but without any limitation, the cells can be T cells, Natural Killer (NK) cells, B cells, dendritic cells, hematopoietic stem cells, or pluripotent stem cells.

In certain embodiments, the cell is autologous. In certain embodiments, the cell is a T cell. Non-limiting examples of T cells encompassed by the present disclosure include helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the T cell is a CD4⁺ T cell. In certain embodiments, the T cell is a CD8⁺ T cell.

In certain embodiments, the T cell is a peripheral T cell. Peripheral T cells are differentiated T cells that have undergone the maturation process. Peripheral T cells can be found in peripheral blood. In certain embodiments, the T cell is not a naïve T cell. Naïve T cells are precursors for effector and memory T cell subsets. Phenotypically, naïve T cells are small cells with little cytoplasm; they express surface markers, such as CD45RA, CCR7, CD62L, CD127, and CD132. Naïve T cells lack expression of markers of previous activation, such as CD25, CD44, CD69, CD45RO, or HLA-DR.

In certain embodiments, the cell is an NK cell. Natural killer (NK) cells can be lymphocytes that are part of cell-mediated immunity and act during the innate immune response. NK cells do not require prior activation to perform their cytotoxic effect on target cells.

2.8.1. Exemplified Cells

In certain embodiments, the cell is a T cell. In certain embodiments, the T cell is a peripheral T cell. In certain embodiments, the cell comprises an exogenous TCR. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCRβ locus. In certain embodiments, the cell further comprises gene disruption of a TRCα locus and a TCRβ locus. In certain embodiments, the TRCα locus is a TRAC locus. In certain embodiments, the TCRβ locus is a TRBC locus.

In certain embodiments, the cell is a T cell. In certain embodiments, the T cell is a peripheral T cell. In certain embodiments, the cell comprises an exogenous TCR and a gene disruption of a TGFβRII locus. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCR locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the gene disruption of a TGFβRII locus generated a knockout of the TGFβRII gene expression. In certain embodiments, the gene disruption of a TGFβRII locus is obtained by using a CRISPR/Cas system. In certain embodiments, the CRISPR/Cas system comprises a gRNA. In certain embodiments, the gRNA comprises the nucleotide sequence set forth in SEQ ID NOs: 1-3.

In certain embodiments, the cell is a T cell. In certain embodiments, the T cell is a peripheral T cell. In certain embodiments, the cell comprises an exogenous TCR and a dominant negative TGFβRII (dnTGFβRII). In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCR locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the dnTGFβRII comprises an amino acid sequence that is at least about 80% identical to the amino acid sequence set forth in SEQ ID NO: 8. In certain embodiments, the dnTGFβRII comprises the amino acid sequence set forth in SEQ ID NO: 8.

In certain embodiments, the cell is a T cell. In certain embodiments, the T cell is a peripheral T cell. In certain embodiments, the cell comprises an exogenous TCR, an exogenous CD8 receptor, and a gene disruption of a TGFβRII locus. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCRβ locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the exogenous CD8 receptor is a homodimer. In certain embodiments, the exogenous CD8 receptor comprises a first monomer and a second monomer. In certain embodiments, the first and second monomers comprise a signal peptide. In certain embodiments, the signal peptide comprises the amino acid sequence set forth in SEQ ID NO: 9. In certain embodiments, the first and second monomers comprise an extracellular domain. In certain embodiments, the extracellular domain comprises the amino acid sequence set forth in SEQ ID NO: 10. In certain embodiments, the first and second monomers comprise a transmembrane domain. In certain embodiments, the transmembrane domain comprises the amino acid sequence set forth in SEQ ID NO: 11. In certain embodiments, the first and second monomers comprise an intracellular domain. In certain embodiments, the intracellular domain comprises the amino acid sequence set forth in SEQ ID NO: 12. In certain embodiments, the gene disruption of a TGFβRII locus generated a knockout of the TGFβRII gene expression. In certain embodiments, the gene disruption of a TGFβRII locus is obtained by using a CRISPR/Cas system. In certain embodiments, the CRISPR/Cas system comprises a gRNA. In certain embodiments, the gRNA comprises the nucleotide sequence set forth in SEQ ID NOs: 1-3.

In certain embodiments, the cell is a T cell. In certain embodiments, the T cell is a peripheral T cell. In certain embodiments, the cell comprises an exogenous TCR, an exogenous CD8 receptor, and a gene disruption of a TGFβRII locus. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCRβ locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the exogenous CD8 receptor is a homodimer. In certain embodiments, the exogenous CD8 receptor comprises a first monomer and a second monomer. In certain embodiments, the first and second monomers comprise a signal peptide. In certain embodiments, the signal peptide comprises the amino acid sequence set forth in SEQ ID NO: 9. In certain embodiments, the first and second monomers comprise an extracellular domain. In certain embodiments, the extracellular domain comprises the amino acid sequence set forth in SEQ ID NO: 10. In certain embodiments, the first and second monomers comprise a transmembrane domain. In certain embodiments, the transmembrane domain comprises the amino acid sequence set forth in SEQ ID NO: 11. In certain embodiments, the first and second monomers comprise an intracellular domain. In certain embodiments, the intracellular domain comprises the amino acid sequence set forth in SEQ ID NO: 16. In certain embodiments, the gene disruption of a TGFβRII locus generated a knockout of the TGFβRII gene expression. In certain embodiments, the gene disruption of a TGFβRII locus is obtained by using a CRISPR/Cas system. In certain embodiments, the CRISPR/Cas system comprises a gRNA. In certain embodiments, the gRNA comprises the nucleotide sequence set forth in SEQ ID NOs: 1-3.

In certain embodiments, the cell is a T cell. In certain embodiments, the T cell is a peripheral T cell. In certain embodiments, the cell comprises an exogenous TCR, an exogenous CD8 receptor, and a gene disruption of a TGFβRII locus. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCRβ locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the exogenous CD8 receptor is a homodimer. In certain embodiments, the exogenous CD8 receptor comprises a first monomer and a second monomer. In certain embodiments, the first and second monomers comprise a signal peptide. In certain embodiments, the signal peptide comprises the amino acid sequence set forth in SEQ ID NO: 9. In certain embodiments, the first and second monomers comprise an extracellular domain. In certain embodiments, the extracellular domain comprises the amino acid sequence set forth in SEQ ID NO: 10. In certain embodiments, the first and second monomers comprise a transmembrane domain. In certain embodiments, the transmembrane domain comprises the amino acid sequence set forth in SEQ ID NO: 11. In certain embodiments, the first and second monomers comprise an intracellular domain. In certain embodiments, the intracellular domain comprises the amino acid sequence set forth in SEQ ID NO: 17. In certain embodiments, the gene disruption of a TGFβRII locus generated a knockout of the TGFβRII gene expression. In certain embodiments, the gene disruption of a TGFβRII locus is obtained by using a CRISPR/Cas system. In certain embodiments, the CRISPR/Cas system comprises a gRNA. In certain embodiments, the gRNA comprises the nucleotide sequence set forth in SEQ ID NOs: 1-3.

In certain embodiments, the cell is a T cell. In certain embodiments, the T cell is a peripheral T cell. In certain embodiments, the cell comprises an exogenous TCR, an exogenous CD8 receptor, and a gene disruption of a TGFβRII locus. In certain embodiments, the exogenous TCR comprises a TRCα sequence and a TCRβ sequence. In certain embodiments, the exogenous TCR is encoded by an exogenous polynucleotide integrated into a TRAC locus. In certain embodiments, the exogenous TCR is encoded by an exogenous polynucleotide integrated into a TRBC locus. In certain embodiments, the cell further comprises gene disruption of a TRCα locus or a TCRβ locus. In certain embodiments, the cell further comprises gene disruption of a TRCα locus and a TCRβ locus. In certain embodiments, the TRCα locus is a TRAC locus. In certain embodiments, the TCRβ locus is a TRBC locus. In certain embodiments, the exogenous CD8 receptor is a heterodimer. In certain embodiments, the exogenous CD8 receptor comprises a first monomer and a second monomer. In certain embodiments, the first monomer comprises a signal peptide. In certain embodiments, the signal peptide comprises the amino acid sequence set forth in SEQ ID NO: 9. In certain embodiments, the first monomer comprises an extracellular domain. In certain embodiments, the extracellular domain comprises the amino acid sequence set forth in SEQ ID NO: 10. In certain embodiments, the first monomer comprises a transmembrane domain. In certain embodiments, the transmembrane domain comprises the amino acid sequence set forth in SEQ ID NO: 11. In certain embodiments, the first monomer comprises an intracellular domain. In certain embodiments, the intracellular domain comprises the amino acid sequence set forth in SEQ ID NO: 12. In certain embodiments, the second monomer comprises a signal peptide. In certain embodiments, the signal peptide comprises the amino acid sequence set forth in SEQ ID NO: 13. In certain embodiments, the second monomer comprises an extracellular domain. In certain embodiments, the extracellular domain comprises the amino acid sequence set forth in SEQ ID NO: 14. In certain embodiments, the second monomer comprises a transmembrane domain. In certain embodiments, the transmembrane domain comprises the amino acid sequence set forth in SEQ ID NO: 15. In certain embodiments, the second monomer comprises an intracellular domain. In certain embodiments, the intracellular domain comprises the amino acid sequence set forth in SEQ ID NO: 16. In certain embodiments, the gene disruption of a TGFβRII locus generated a knockout of the TGFβRII gene expression. In certain embodiments, the gene disruption of a TGFβRII locus is obtained by using a CRISPR/Cas system. In certain embodiments, the CRISPR/Cas system comprises a gRNA. In certain embodiments, the gRNA comprises the nucleotide sequence set forth in SEQ ID NOs: 1-3.

### 2.9. Pharmaceutical Formulations

Pharmaceutical formulations of the TFGβRII Products, dnTGFBRII Products, and CD8-TGFβRII Products are prepared by combining the respective TFGβRII, dnTGFBRII, or CD8-TGFβRII Cells in a solution that can preserve the ‘young’ phenotype of the cells in a cryopreserved state. Table 4 (as disclosed in Section 5 below) provides an example of one such pharmaceutical formulation. Alternatively, pharmaceutical formulations of the TFGβRII Products, dnTGFBRII Products, and CD8-TGFβRII Products can be prepared by combining the respective TFGβRII, dnTGFBRII, or CD8-TGFβRII Cells in a solution that can preserve the ‘young’ phenotype of the cells without the need to freeze or cryopreserve the product (i.e., the TFGβRII Products, dnTGFBRII Products, or CD8-TGFβRII Products is maintained in an aqueous solution or as a non-frozen/cryopreserved cell pellet).

Additional pharmaceutically acceptable carriers, buffers, stabilizers, and/or preservatives can also be added to the cryopreservation solution or the aqueous storage solution (if the TFGβRII Product, dnTGFBRII Product, or CD8-TGFβRII Product is not cryopreserved). Any cryopreservation agent and/or media can be used to cryopreserve the TFGβRII Products, dnTGFBRII Products, and CD8-TGFβRII Products including but not limited to CryoStor, CryoStor CS5, CELLBANKER, and custom cryopreservation media that optionally include DMSO.

### 3. Methods of Treatment

The presently disclosed subject matter provides methods for inducing and/or increasing an immune response in a subject in need thereof. The TFGβRII Products, dnTGFBRII Products, and CD8-TGFβRII Products can be used for treating and/or preventing a cancer in a subject. The TFGβRII Products, dnTGFBRII Products, and CD8-TGFβRII Products can be used for prolonging the survival of a subject suffering from a cancer. The TFGβRII Products, dnTGFBRII Products, and CD8-TGFβRII Products can also be used for treating and/or preventing a cancer in a subject. The TFGβRII Products, dnTGFBRII Products, and CD8-TGFβRII Products can also be used for reducing tumor burden in a subject. Such methods comprise administering the TFGβRII Products, dnTGFBRII Products, or CD8-TGFβRII Products in an amount effective or a composition (e.g., a pharmaceutical composition) comprising TFGβRII Cells, dnTGFBRII Cells, or CD8-TGFβRII Cells to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the TFGβRII Products, dnTGFBRII Products, and CD8-TGFβRII Products can be used for treating viral or bacterial diseases. In certain embodiments, the TFGβRII Products, dnTGFBRII Products, and CD8-TGFβRII Products can be used for treating autoimmune diseases.

In certain embodiments, an effective amount of the TFGβRII Products, dnTGFBRII Products, and CD8-TGFβRII Products are delivered through intravenous (IV) administration. In certain embodiments, the TFGβRII Products, dnTGFBRII Products, and CD8-TGFβRII Products are delivered through intravenous (IV) administration in a single administration. In certain embodiments, the TFGβRII Products, dnTGFBRII Products, and CD8-TGFβRII Products are delivered through intravenous (IV) administration in multiple administrations. In certain embodiments, the TFGβRII Products, dnTGFBRII Products, and CD8-TGFβRII Products are delivered through intravenous (IV) administration in two or more administrations. In certain embodiments, the TFGβRII Products, dnTGFBRII Products, and CD8-TGFβRII Products are delivered through intravenous (IV) administration in two administrations. In certain embodiments, the TFGβRII Products, dnTGFBRII Products, and CD8-TGFβRII Products are delivered through intravenous (IV) administration in three administrations.

The presently disclosed subject matter provides methods for treating and/or preventing cancer in a subject. In certain embodiments, the method comprises administering an effective amount of TFGβRII Products, dnTGFBRII Products, or CD8-TGFβRII Products to a subject having cancer.

Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed young T cells and compositions comprising thereof can be used for treating and/or preventing blood cancers (e.g., leukemias, lymphomas, and myelomas) or ovarian cancer, which are not amenable to conventional therapeutic interventions.

In certain embodiments, the cancer is a solid cancer. In certain embodiments, the solid cancer is selected from the group consisting of glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram, or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

### 4. Articles of Manufacture

The TFGβRII Products, dnTGFBRII Products, and CD8-TGFβRII Products can be used in combination with articles of manufacture. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. The containers may be made of any material that is acceptable for the storage and preservation of the TFGβRII Cells, dnTGFBRII Cells, and CD8-TGFβRII Cells within their respective TFGβRII Products, dnTGFBRII Products, and CD8-TGFβRII Products. In certain embodiments, the container may be an intravenous solution bag or a vial having a stopper pierceable by a hypodermic injection needle. For example, the container may be a CryoMACS freezing bag. The label or package insert indicates that the TFGβRII Products, dnTGFBRII Products, and CD8-TGFβRII Products are used for treating the condition of choice and the patient of origin. The patient is identified on the container of the TFGβRII Products, dnTGFBRII Products, and CD8-TGFβRII Products because the TFGβRII Products, dnTGFBRII Products, and CD8-TGFβRII Products are made from autologous cells and engineered as a patient-specific and individualized treatment.

The article of manufacture may comprise: 1) a first container with a TGFβRII Product contained therein.

The article of manufacture may comprise: 1) a first container with a TGFβRII Product contained therein; and 2) a second container with the same TGFβRII Product as the first container contained therein. Optionally, additional containers with the same TGFβRII Product as the first and second containers may be prepared and made. Optionally, additional containers containing a composition comprising a different cytotoxic or otherwise therapeutic agent may also be combined with the containers described above.

The article of manufacture may comprise: 1) a first container with a TGFβRII Product contained therein; and 2) a second container with a composition contained therein, wherein the composition comprises a further cytotoxic or otherwise therapeutic agent.

The article of manufacture may comprise: 1) a first container with two TGFβRII Products contained therein; and 2) a second container with a composition contained therein, wherein the composition comprises a further cytotoxic or otherwise therapeutic agent.

The article of manufacture may comprise: 1) a first container with a TGFβRII Product contained therein; 2) a second container with a second TGFβRII Product contained therein; and 3) optionally a third container with a composition contained therein, wherein the composition comprises a further cytotoxic or otherwise therapeutic agent. In certain embodiments, the first and second TGFβRII Products are different TGFβRII Products. In certain embodiments, the first and second TGFβRII Products are the same TGFβRII Products.

The article of manufacture may comprise: 1) a first container with three TGFβRII Products contained therein; and 2) optionally a second container with a composition contained therein, wherein the composition comprises a further cytotoxic or otherwise therapeutic agent.

The article of manufacture may comprise: 1) a first container with a TGFβRII Product contained therein; 2) a second container with a second TGFβRII Product contained therein; 3) a third container with a third TGFβRII Product contained therein; and 4) optionally a fourth container with a composition contained therein, wherein the composition comprises a further cytotoxic or otherwise therapeutic agent. In certain embodiments, the first, second, and third TGFβRII Products are different TGFβRII Products. In certain embodiments, the first, second, and third TGFβRII Products are the same TGFβRII Products. In certain embodiments, two of the first, second, and third TGFβRII Products are the same TGFβRII Products.

The article of manufacture may comprise: 1) a first container with four TGFβRII Products contained therein; and 2) optionally a second container with a composition contained therein, wherein the composition comprises a further cytotoxic or otherwise therapeutic agent.

The article of manufacture may comprise: 1) a first container with a TGFβRII Product contained therein; 2) a second container with a second TGFβRII Product contained therein; 3) a third container with a third TGFβRII Product contained therein; 4) a fourth container with a fourth TGFβRII Product contained therein; and 5) optionally a fifth container with a composition contained therein, wherein the composition comprises a further cytotoxic or otherwise therapeutic agent. In certain embodiments, the first, second, third, and fourth TGFβRII Products are different TGFβRII Products. In certain embodiments, the first, second, third, and fourth TGFβRII Products are the same NeoTCR Products. In certain embodiments, two of the first, second, third, and fourth TGFβRII Products are the same NeoTCR Products. In certain embodiments, three of the first, second, third, and fourth TGFβRII Products are the same TGFβRII Products.

The article of manufacture may comprise: 1) a first container with five or more TGFβRII Products contained therein; and 2) optionally a second container with a composition contained therein, wherein the composition comprises a further cytotoxic or otherwise therapeutic agent.

The article of manufacture may comprise: 1) a first container with a TGFβRII Product contained therein; 2) a second container with a second TGFβRII Product contained therein; 3) a third container with a third TGFβRII Product contained therein; 4) a fourth container with a fourth TGFβRII Product contained therein; 5) a fifth container with a fifth TGFβRII Product contained therein; 6) optionally a sixth or more additional containers with a sixth or more TGFβRII Product contained therein; and 7) optionally an additional container with a composition contained therein, wherein the composition comprises a further cytotoxic or otherwise therapeutic agent. In certain embodiments, all of the containers of TGFβRII Products are different TGFβRII Products. In certain embodiments, all of the containers of TGFβRII Products are the same TGFβRII Products. In certain embodiments, there can be any combination of same or different TGFβRII Products in the five or more containers based on the availability of detectable CD8s in a patient's tumor sample(s), the need and/or desire to have multiple TGFβRII Products for the patient, and the availability of any one TGFβRII Product that may require or benefit from one or more container.

The article of manufacture may comprise: 1) a first container with a TGFβRII Product contained therein; 2) a second container with a second TGFβRII Product contained therein; and 3) a third container with a third TGFβRII Product contained therein.

The article of manufacture may comprise: 1) a first container with a TGFβRII Product contained therein; 2) a second container with a second TGFβRII Product contained therein; 3) a third container with a third TGFβRII Product contained therein; and 4) optionally a fourth container with a fourth TGFβRII Product contained therein.

The article of manufacture may comprise: 1) a first container with a TGFβRII Product contained therein; 2) a second container with a second TGFβRII Product contained therein; 3) a third container with a third TGFβRII Product contained therein; 4) a fourth container with a fourth TGFβRII Product contained therein; and 5) optionally a fifth container with a fourth TGFβRII Product contained therein.

The article of manufacture may comprise a container with one TGFβRII Product contained therein. The article of manufacture may comprise a container with two TGFβRII Products contained therein. The article of manufacture may comprise a container with three TGFβRII Products contained therein. The article of manufacture may comprise a container with four TGFβRII Products contained therein. The article of manufacture may comprise a container with five TGFβRII Products contained therein.

The article of manufacture may comprise 1) a first container with one TGFβRII Product contained therein, and 2) a second container with two TGFβRII Products contained therein. The article of manufacture may comprise 1) a first container with two TGFβRII Products contained therein, and 2) a second container with one TGFβRII Product contained therein. In the examples above, a third and/or fourth container comprising one or more additional TGFβRII Products may be included in the article of manufacture. Additionally, a fifth container comprising one or more additional TGFβRII Products may be included in the article of manufacture.

Furthermore, any container of TGFβRII Product described herein can be split into two, three, or four separate containers for multiple time points of administration and/or based on the appropriate dose for the patient.

In certain embodiments, the TGFβRII Products are provided in a kit. The kit can, by means of non-limiting examples, contain package insert(s), labels, instructions for using the TGFβRII Product(s), syringes, disposal instructions, administration instructions, tubing, needles, and anything else a clinician would need in order to properly administer the TGFβRII Product(s).

The article of manufacture may comprise: 1) a first container with a dnTGFβRII Product contained therein.

The article of manufacture may comprise: 1) a first container with a dnTGFβRII Product contained therein; and 2) a second container with the same dnTGFβRII Product as the first container contained therein. Optionally, additional containers with the same dnTGFβRII Product as the first and second containers may be prepared and made. Optionally, additional containers containing a composition comprising a different cytotoxic or otherwise therapeutic agent may also be combined with the containers described above.

The article of manufacture may comprise: 1) a first container with a dnTGFβRII Product contained therein; and 2) a second container with a composition contained therein, wherein the composition comprises a further cytotoxic or otherwise therapeutic agent.

The article of manufacture may comprise: 1) a first container with two dnTGFβRII Products contained therein; and 2) a second container with a composition contained therein, wherein the composition comprises a further cytotoxic or otherwise therapeutic agent.

The article of manufacture may comprise: 1) a first container with a dnTGFβRII Product contained therein; 2) a second container with a second dnTGFβRII Product contained therein; and 3) optionally a third container with a composition contained therein, wherein the composition comprises a further cytotoxic or otherwise therapeutic agent. In certain embodiments, the first and second dnTGFβRII Products are different dnTGFβRII Products. In certain embodiments, the first and second dnTGFβRII Products are the same dnTGFβRII Products.

The article of manufacture may comprise: 1) a first container with three dnTGFβRII Products contained therein; and 2) optionally a second container with a composition contained therein, wherein the composition comprises a further cytotoxic or otherwise therapeutic agent.

The article of manufacture may comprise: 1) a first container with a dnTGFβRII Product contained therein; 2) a second container with a second dnTGFβRII Product contained therein; 3) a third container with a third dnTGFβRII Product contained therein; and 4) optionally a fourth container with a composition contained therein, wherein the composition comprises a further cytotoxic or otherwise therapeutic agent. In certain embodiments, the first, second, and third dnTGFβRII Products are different dnTGFβRII Products. In certain embodiments, the first, second, and third dnTGFβRII Products are the same dnTGFβRII Products. In certain embodiments, two of the first, second, and third dnTGFβRII Products are the same dnTGFβRII Products.

The article of manufacture may comprise: 1) a first container with four dnTGFβRII Products contained therein; and 2) optionally a second container with a composition contained therein, wherein the composition comprises a further cytotoxic or otherwise therapeutic agent.

The article of manufacture may comprise: 1) a first container with a dnTGFβRII Product contained therein; 2) a second container with a second dnTGFβRII Product contained therein; 3) a third container with a third dnTGFβRII Product contained therein; 4) a fourth container with a fourth dnTGFβRII Product contained therein; and 5) optionally a fifth container with a composition contained therein, wherein the composition comprises a further cytotoxic or otherwise therapeutic agent. In certain embodiments, the first, second, third, and fourth dnTGFβRII Products are different dnTGFβRII Products. In certain embodiments, the first, second, third, and fourth dnTGFβRII Products are the same NeoTCR Products. In certain embodiments, two of the first, second, third, and fourth dnTGFβRII Products are the same NeoTCR Products. In certain embodiments, three of the first, second, third, and fourth dnTGFβRII Products are the same dnTGFβRII Products.

The article of manufacture may comprise: 1) a first container with five or more dnTGFβRII Products contained therein; and 2) optionally a second container with a composition contained therein, wherein the composition comprises a further cytotoxic or otherwise therapeutic agent.

The article of manufacture may comprise: 1) a first container with a dnTGFβRII Product contained therein; 2) a second container with a second dnTGFβRII Product contained therein; 3) a third container with a third dnTGFβRII Product contained therein; 4) a fourth container with a fourth dnTGFβRII Product contained therein; 5) a fifth container with a fifth dnTGFβRII Product contained therein; 6) optionally a sixth or more additional containers with a sixth or more dnTGFβRII Product contained therein; and 7) optionally an additional container with a composition contained therein, wherein the composition comprises a further cytotoxic or otherwise therapeutic agent. In certain embodiments, all of the containers of dnTGFβRII Products are different dnTGFβRII Products. In certain embodiments, all of the containers of dnTGFβRII Products are the same dnTGFβRII Products. In certain embodiments, there can be any combination of same or different dnTGFβRII Products in the five or more containers based on the availability of detectable CD8s in a patient's tumor sample(s), the need and/or desire to have multiple dnTGFβRII Products for the patient, and the availability of any one dnTGFβRII Product that may require or benefit from one or more container.

The article of manufacture may comprise: 1) a first container with a dnTGFβRII Product contained therein; 2) a second container with a second dnTGFβRII Product contained therein; and 3) a third container with a third dnTGFβRII Product contained therein.

The article of manufacture may comprise: 1) a first container with a dnTGFβRII Product contained therein; 2) a second container with a second dnTGFβRII Product contained therein; 3) a third container with a third dnTGFβRII Product contained therein; and 4) optionally a fourth container with a fourth dnTGFβRII Product contained therein.

The article of manufacture may comprise: 1) a first container with a dnTGFβRII Product contained therein; 2) a second container with a second dnTGFβRII Product contained therein; 3) a third container with a third dnTGFβRII Product contained therein; 4) a fourth container with a fourth dnTGFβRII Product contained therein; and 5) optionally a fifth container with a fourth dnTGFβRII Product contained therein.

The article of manufacture may comprise a container with one dnTGFβRII Product contained therein. The article of manufacture may comprise a container with two dnTGFβRII Products contained therein. The article of manufacture may comprise a container with three dnTGFβRII Products contained therein. The article of manufacture may comprise a container with four dnTGFβRII Products contained therein. The article of manufacture may comprise a container with five dnTGFβRII Products contained therein.

The article of manufacture may comprise 1) a first container with one dnTGFβRII Product contained therein, and 2) a second container with two dnTGFβRII Products contained therein. The article of manufacture may comprise 1) a first container with two dnTGFβRII Products contained therein, and 2) a second container with one dnTGFβRII Product contained therein. In the examples above, a third and/or fourth container comprising one or more additional dnTGFβRII Products may be included in the article of manufacture. Additionally, a fifth container comprising one or more additional dnTGFβRII Products may be included in the article of manufacture.

Furthermore, any container of dnTGFβRII Product described herein can be split into two, three, or four separate containers for multiple time points of administration and/or based on the appropriate dose for the patient.

In certain embodiments, the dnTGFβRII Products are provided in a kit. The kit can, by means of non-limiting examples, contain package insert(s), labels, instructions for using the dnTGFβRII Product(s), syringes, disposal instructions, administration instructions, tubing, needles, and anything else a clinician would need in order to properly administer the dnTGFβRII Product(s).

The article of manufacture may comprise: 1) a first container with a CD8-TGFβRII Product contained therein.

The article of manufacture may comprise: 1) a first container with a CD8-TGFβRII Product contained therein; and 2) a second container with the same CD8-TGFβRII Product as the first container contained therein. Optionally, additional containers with the same CD8-TGFβRII Product as the first and second containers may be prepared and made. Optionally, additional containers containing a composition comprising a different cytotoxic or otherwise therapeutic agent may also be combined with the containers described above.

The article of manufacture may comprise: 1) a first container with a CD8-TGFβRII Product contained therein; and 2) a second container with a composition contained therein, wherein the composition comprises a further cytotoxic or otherwise therapeutic agent.

The article of manufacture may comprise: 1) a first container with two CD8-TGFβRII Products contained therein; and 2) a second container with a composition contained therein, wherein the composition comprises a further cytotoxic or otherwise therapeutic agent.

The article of manufacture may comprise: 1) a first container with a CD8-TGFβRII Product contained therein; 2) a second container with a second CD8-TGFβRII Product contained therein; and 3) optionally a third container with a composition contained therein, wherein the composition comprises a further cytotoxic or otherwise therapeutic agent. In certain embodiments, the first and second CD8-TGFβRII Products are different CD8-TGFβRII Products. In certain embodiments, the first and second CD8-TGFβRII Products are the same CD8-TGFβRII Products.

The article of manufacture may comprise: 1) a first container with three CD8-TGFβRII Products contained therein; and 2) optionally a second container with a composition contained therein, wherein the composition comprises a further cytotoxic or otherwise therapeutic agent.

The article of manufacture may comprise: 1) a first container with a CD8-TGFβRII Product contained therein; 2) a second container with a second CD8-TGFβRII Product contained therein; 3) a third container with a third CD8-TGFβRII Product contained therein; and 4) optionally a fourth container with a composition contained therein, wherein the composition comprises a further cytotoxic or otherwise therapeutic agent. In certain embodiments, the first, second, and third CD8-TGFβRII Products are different CD8-TGFβRII Products. In certain embodiments, the first, second, and third CD8-TGFβRII Products are the same CD8-TGFβRII Products. In certain embodiments, two of the first, second, and third CD8-TGFβRII Products are the same CD8-TGFβRII Products.

The article of manufacture may comprise: 1) a first container with four CD8-TGFβRII Products contained therein; and 2) optionally a second container with a composition contained therein, wherein the composition comprises a further cytotoxic or otherwise therapeutic agent.

The article of manufacture may comprise: 1) a first container with a CD8-TGFβRII Product contained therein; 2) a second container with a second CD8-TGFβRII Product contained therein; 3) a third container with a third CD8-TGFβRII Product contained therein; 4) a fourth container with a fourth CD8-TGFβRII Product contained therein; and 5) optionally a fifth container with a composition contained therein, wherein the composition comprises a further cytotoxic or otherwise therapeutic agent. In certain embodiments, the first, second, third, and fourth CD8-TGFβRII Products are different CD8-TGFβRII Products. In certain embodiments, the first, second, third, and fourth CD8-TGFβRII Products are the same NeoTCR Products. In certain embodiments, two of the first, second, third, and fourth CD8-TGFβRII Products are the same NeoTCR Products. In certain embodiments, three of the first, second, third, and fourth CD8-TGFβRII Products are the same CD8-TGFβRII Products.

The article of manufacture may comprise: 1) a first container with five or more CD8-TGFβRII Products contained therein; and 2) optionally a second container with a composition contained therein, wherein the composition comprises a further cytotoxic or otherwise therapeutic agent.

The article of manufacture may comprise: 1) a first container with a CD8-TGFβRII Product contained therein; 2) a second container with a second CD8-TGFβRII Product contained therein; 3) a third container with a third CD8-TGFβRII Product contained therein; 4) a fourth container with a fourth CD8-TGFβRII Product contained therein; 5) a fifth container with a fifth CD8-TGFβRII Product contained therein; 6) optionally a sixth or more additional containers with a sixth or more CD8-TGFβRII Product contained therein; and 7) optionally an additional container with a composition contained therein, wherein the composition comprises a further cytotoxic or otherwise therapeutic agent. In certain embodiments, all of the containers of CD8-TGFβRII Products are different CD8-TGFβRII Products. In certain embodiments, all of the containers of CD8-TGFβRII Products are the same CD8-TGFβRII Products. In certain embodiments, there can be any combination of same or different CD8-TGFβRII Products in the five or more containers based on the availability of detectable CD8s in a patient's tumor sample(s), the need and/or desire to have multiple CD8-TGFβRII Products for the patient, and the availability of any one CD8-TGFβRII Product that may require or benefit from one or more container.

The article of manufacture may comprise: 1) a first container with a CD8-TGFβRII Product contained therein; 2) a second container with a second CD8-TGFβRII Product contained therein; and 3) a third container with a third CD8-TGFβRII Product contained therein.

The article of manufacture may comprise: 1) a first container with a CD8-TGFβRII Product contained therein; 2) a second container with a second CD8-TGFβRII Product contained therein; 3) a third container with a third CD8-TGFβRII Product contained therein; and 4) optionally a fourth container with a fourth CD8-TGFβRII Product contained therein.

The article of manufacture may comprise: 1) a first container with a CD8-TGFβRII Product contained therein; 2) a second container with a second CD8-TGFβRII Product contained therein; 3) a third container with a third CD8-TGFβRII Product contained therein; 4) a fourth container with a fourth CD8-TGFβRII Product contained therein; and 5) optionally a fifth container with a fourth CD8-TGFβRII Product contained therein.

The article of manufacture may comprise a container with one CD8-TGFβRII Product contained therein. The article of manufacture may comprise a container with two CD8-TGFβRII Products contained therein. The article of manufacture may comprise a container with three CD8-TGFβRII Products contained therein. The article of manufacture may comprise a container with four CD8-TGFβRII Products contained therein. The article of manufacture may comprise a container with five CD8-TGFβRII Products contained therein.

The article of manufacture may comprise 1) a first container with one CD8-TGFβRII Product contained therein, and 2) a second container with two CD8-TGFβRII Products contained therein. The article of manufacture may comprise 1) a first container with two CD8-TGFβRII Products contained therein, and 2) a second container with one CD8-TGFβRII Product contained therein. In the examples above, a third and/or fourth container comprising one or more additional CD8-TGFβRII Products may be included in the article of manufacture. Additionally, a fifth container comprising one or more additional CD8-TGFβRII Products may be included in the article of manufacture.

Furthermore, any container of CD8-TGFβRII Product described herein can be split into two, three, or four separate containers for multiple time points of administration and/or based on the appropriate dose for the patient.

In certain embodiments, the CD8-TGFβRII Products are provided in a kit. The kit can, by means of non-limiting examples, contain package insert(s), labels, instructions for using the CD8-TGFβRII Product(s), syringes, disposal instructions, administration instructions, tubing, needles, and anything else a clinician would need in order to properly administer the CD8-TGFβRII Product(s).

### 5. Therapeutic Compositions and Methods of Manufacturing

As described herein, plasmid DNA-mediated precision genome engineering process for Good Manufacturing Practice (GMP) manufacturing of TGFβRII Products, dnTGFβRII Products, and CD8-TGFβRII Products were developed. Targeted integration of the patient-specific NeoTCR was accomplished by electroporating CRISPR endonuclease ribonucleoproteins (RNPs) together with the personalized NeoTCR gene cassette, encoded by the plasmid DNA. In addition to the NeoTCR, the CD8 Constructs and dnTGFβRII Constructs were inserted by incorporating them into the NeoTCR vector and then electroporating with CRISPR endonuclease ribonucleoproteins (RNPs) as described above.

Each of the TGFβRII Products, dnTGFβRII Products, and CD8-TGFβRII Products can each be individually formulated into a drug product using the clinical manufacturing process. Under this process, the GFβRII Products, dnTGFβRII Products, and CD8-TGFβRII Products are cryopreserved in CryoMACS Freezing Bags. One or more bags may be shipped to the site for each patient depending on patient needs. The product is composed of apheresis-derived, patient-autologous, CD8 and CD4 T cells that have been precision genome engineered to express one or more autologous NeoTCRs targeting a neoepitope complexed to one of the endogenous HLA receptors presented exclusively on the surface of that patient's tumor cells.

The final product will contain 5% dimethyl sulfoxide (DMSO), human serum albumin, and Plasma-Lyte. The final cell product will contain the list of components provided in Table 4.

TABLE 4
Exemplary Composition of the TGFβRII Products,
dnTGFβRII Products, and CD8-TGFβRII Products

| Component | Specification/Grade |
|---|---|
| Total nucleated NeoTCR cells | cGMP manufactured |
| Plasma-Lyte A | USP |
| Human Serum Albumin in | USP |
| 0.02-0.08M sodium caprylate | |
| and sodium tryptophanate | |
| CryoStor CS10 | cGMP manufactured |
| | with USP grade materials |

### 6. Kits

The presently disclosed subject matter provides kits for inducing and/or enhancing an immune response and/or treating and/or preventing a cancer or a pathogen infection in a subject. In certain embodiments, the kit comprises an effective amount of presently disclosed cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed HR template.

If desired, the cells and/or nucleic acid molecules are provided together with instructions for administering the cells or nucleic acid molecules to a subject having or at risk of developing a cancer or pathogen or immune disorder. The instructions generally include information about the use of the composition for the treatment and/or prevention of a cancer or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container. The resulting cells can be grown under conditions similar to those for unmodified cells, whereby the modified cells can be expanded and used for a variety of purposes.

### 7. Exemplary Embodiments

In certain embodiments, the present disclosure provides an immune cell, comprising:
a) an exogenous T cell receptor (TCR);
b) an exogenous CD8 receptor; and
c) a gene disruption of a TGFβRII locus.

In certain embodiments, the present disclosure provides an immune cell, comprising:
a) an exogenous polynucleotide comprising a sequence encoding an exogenous TCR and a sequence encoding an exogenous CD8 receptor; and
b) a gene disruption of a TGFβRII locus;
wherein the exogenous polynucleotide is integrated at a TRAC or TRBC locus, and wherein the sequence encoding an exogenous TCR and the sequence encoding an exogenous CD8 receptor are under control of a TRAC or TRBC promoter.

In certain embodiments, the present disclosure provides an immune cell, comprising an exogenous TCR and a gene disruption of a TGFβRII locus.

In certain embodiments, the present disclosure provides an immune cell, comprising an exogenous polynucleotide comprising a sequence encoding an exogenous TCR and a gene disruption of a TGFβRII locus, wherein the exogenous polynucleotide is integrated at a TRAC or TRBC locus, and wherein the sequence encoding an exogenous TCR is under control of a TRAC or TRBC promoter.

In certain embodiments of the cells disclosed herein, the exogenous CD8 receptor comprises a first monomer and a second monomer. In certain embodiments of the cells disclosed herein, the first monomer and the second monomer are the same. In certain embodiments of the cells disclosed herein, the first monomer and the second monomer are different. In certain embodiments of the cells disclosed herein, each of the first monomer and the second monomer comprise a signal peptide, an extracellular domain, a transmembrane domain, and an intracellular domain. In certain embodiments of the cells disclosed herein, the extracellular domain comprises a CD8α extracellular domain or a CD8β extracellular domain. In certain embodiments of the cells disclosed herein, the signal peptide comprises a CD8α signal peptide or a CD8β signal peptide. In certain embodiments of the cells disclosed herein, the transmembrane domain comprises a CD8α transmembrane domain or a CD8β transmembrane domain. In certain embodiments of the cells disclosed herein, the intracellular domain is a CD8α intracellular domain, a CD8β intracellular domain, or a CD4 intracellular domain. In certain embodiments of the cells disclosed herein,
a) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8α signal peptide, an extracellular domain comprising a CD8α, a transmembrane domain comprising a CD8α, and an intracellular domain comprising a CD8α;
b) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8β signal peptide, an extracellular domain comprising a CD8β, a transmembrane domain comprising a CD8β, and an intracellular domain comprising a CD8β;
c) the first monomer comprises a signal peptide comprising a CD8α signal peptide, an extracellular domain comprising a CD8α, a transmembrane domain comprising a CD8α, and an intracellular domain comprising a CD8β; and the second monomer comprises a signal peptide comprising a CD8α signal peptide, an extracellular domain comprising a CD8α, a transmembrane domain comprising a CD8α, and an intracellular domain comprising a CD8β; or
d) the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments of the cells disclosed herein, the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments of the cells disclosed herein, the extracellular domain comprises a) an amino acid sequence that is at least about 80% identical to the amino acid sequence set forth in SEQ ID NO: 10; or b) an amino acid sequence that is at least about 80% identical to the amino acid sequence set forth in SEQ ID NO: 14.

In certain embodiments of the cells disclosed herein, the signal peptide comprises a) an amino acid sequence that is at least about 80% identical to the amino acid sequence set forth in SEQ ID NO: 9; or b) an amino acid sequence that is at least about 80% identical to the amino acid sequence set forth in SEQ ID NO: 13.

In certain embodiments of the cells disclosed herein, the transmembrane domain comprises a) an amino acid sequence that is at least about 80% identical to the amino acid sequence set forth in SEQ ID NO: 11; or b) an amino acid sequence that is at least about 80% identical to the amino acid sequence set forth in SEQ ID NO: 15.

In certain embodiments of the cells disclosed herein, the intracellular domain comprises a) an amino acid sequence that is at least about 80% identical to the amino acid sequence set forth in SEQ ID NO: 12; b) comprises an amino acid sequence that is at least about 80% identical to the amino acid sequence set forth in SEQ ID NO: 16; or c) an amino acid sequence that is at least about 80% identical to the amino acid sequence set forth in SEQ ID NO: 17.

In certain embodiments of the cells disclosed herein,
a) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12;
b) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 13, an extracellular domain comprising an amino acid sequence that is at least about 80% identical to the amino acid sequence set forth in SEQ ID NO: 14, a transmembrane domain comprising an amino acid sequence that is at least about 80% identical to the amino acid sequence set forth in SEQ ID NO: 15, and an intracellular domain comprising an amino acid sequence that is at least about 80% identical to the amino acid sequence set forth in SEQ ID NO: 16;
c) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; or
d) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments of the cells disclosed herein, the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments of the cells disclosed herein,
a) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12;
b) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 12; and the second monomer comprises a signal peptide comprising the amino acid sequence set forth in SEQ ID NO: 13, an extracellular domain comprising the amino acid sequence set forth in SEQ ID NO: 14, a transmembrane domain comprising the amino acid sequence set forth in SEQ ID NO: 15, and an intracellular domain comprising the amino acid sequence set forth in SEQ ID NO: 16;
c) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 16; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 16; or
d) the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17.

In certain embodiments of the cells disclosed herein, the first monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17; and the second monomer comprises a signal peptide comprising the amino acid sequence set forth in SEQ ID NO: 9, an extracellular domain comprising the amino acid sequence set forth in SEQ ID NO: 10, a transmembrane domain comprising the amino acid sequence set forth in SEQ ID NO: 11, and an intracellular domain comprising the amino acid sequence set forth in SEQ ID NO: 17.

In certain embodiments of the cells disclosed herein, the gene disruption of the TGFβRII locus generates a knockout of the TGFβRII gene expression. In certain embodiments of the cells disclosed herein, the gene disruption of the TGFβRII locus is generated by using a CRISPR/Cas system. In certain embodiments of the cells disclosed herein, the CRISPR/Cas system comprises a gRNA. In certain embodiments of the cells disclosed herein, the gRNA comprises a nucleic acid sequence set forth in SEQ ID NOs: 1-3.

In certain embodiments of the cells disclosed herein, the gene disruption of the TGFβRII locus generates a knockdown of the TGFβRII gene expression.

In certain embodiments of the cells disclosed herein, the sequence encoding an exogenous TCR comprises a TCRα gene sequence and a TCRβ gene sequence. In certain embodiments of the cells disclosed herein, the sequence encoding an exogenous TCR further comprises a sequence encoding a protease cleavage site, a sequence encoding a 2A peptide, a sequence encoding a signal peptide, or a combination thereof. In certain embodiments of the cells disclosed herein, the exogenous polynucleotide further comprises a sequence encoding a protease cleavage site, a sequence encoding a 2A peptide, or a combination thereof.

In certain embodiments of the cells disclosed herein, the exogenous polynucleotide, from 5′ to 3′, comprises:
a) a first sequence encoding a 2A peptide, a sequence encoding an exogenous CD8 receptor, a first sequence encoding a protease cleavage site, a second sequence encoding a 2A peptide, a first sequence encoding a signal peptide, a TCRα gene sequence, a sequence encoding a protease cleavage site, a third sequence encoding a 2A peptide, and a TCRβ gene sequence; or
b) a first sequence encoding a 2A peptide, a sequence encoding an exogenous CD8 receptor, a first sequence encoding a protease cleavage site, a second sequence encoding a 2A peptide, a first sequence encoding a signal peptide, a TCRβ gene sequence, a sequence encoding a protease cleavage site, a third sequence encoding a 2A peptide, and a TCRα gene sequence.

In certain embodiments of the cells disclosed herein, the first, second, and third sequences encoding a 2A peptide are codon diverged. In certain embodiments of the cells disclosed herein, the first, second, and third 2A peptide comprise the amino acid sequence set forth in SEQ ID NO: 19. In certain embodiments of the cells disclosed herein, the first and second sequences encoding a protease cleavage site are codon diverged. In certain embodiments of the cells disclosed herein, the first and second sequences encoding a protease cleavage site comprise the amino acid sequence set forth in SEQ ID NOs: 4-7. In certain embodiments of the cells disclosed herein, the first and second sequences encoding a signal peptide are codon diverged. In certain embodiments of the cells disclosed herein, the first and second sequences encoding a signal peptide comprise the amino acid sequence set forth in SEQ ID NO: 35.

In certain embodiments of the cells disclosed herein, the immune cell further comprises a gene disruption of a TRAC locus and a TRBC locus.

In certain embodiments of the cells disclosed herein, the exogenous TCR is a patient derived TCR. In certain embodiments of the cells disclosed herein, the exogenous TCR recognizes a patient derived cancer antigen. In certain embodiments of the cells disclosed herein, the cancer antigen is a neoantigen. In certain embodiments of the cells disclosed herein, the cancer antigen is a private neoantigen.

In certain embodiments of the cells disclosed herein, the immune cell is a patient-derived primary cell. In certain embodiments of the cells disclosed herein, the immune cell is a T cell, a Natural Killer T cell, a Natural Killer cell, or a lymphocyte. In certain embodiments of the cells disclosed herein, the immune cell is a T cell. In certain embodiments of the cells disclosed herein, the cell is a young T cell. In certain embodiments of the cells disclosed herein, the young T cell is a) CD45RA+, CD62L+, CD28+, CD95−, CCR7+, and CD27+; b) CD45RA+, CD62L+, CD28+, CD95+, CD27+, CCR7+; or c) CD45RO+, CD62L+, CD28+, CD95+, CCR7+, CD27+, CD127+.

In certain embodiments of the cells disclosed herein, the exogenous TCR is a CD8-dependent TCR or a CD8-independent TCR.

In certain embodiments of the cells disclosed herein, the immune cell further comprises a gene modification to enhance cell persistence and/or enhances memory cell differentiation.

In certain embodiments of the cells disclosed herein, killing activity of the cell is increased between about 10% to about 500% as compared to killing activity of a cell that does not have the exogenous CD8. In certain embodiments of the cells disclosed herein, proliferation of the cell upon binding of the TCR to the antigen is increased between about 10% to about 500% as compared to proliferation of a cell that does not have the exogenous CD8. In certain embodiments of the cells disclosed herein, secretion of pro-inflammatory cytokine upon binding of the TCR to the antigen by the cell is increased between about 10% to about 500% as compared to secretion by a cell that does not have the exogenous CD8. In certain embodiments of the cells disclosed herein, LCK affinity of the cell is increased between about 10% to about 500% as compared to LCK affinity of a cell that does not have the exogenous CD8. In certain embodiments of the cells disclosed herein, persistence of the cell is increased between about 10% to about 500% as compared to persistence of a cell that does not have the exogenous CD8. In certain embodiments of the cells disclosed herein, tumor infiltration ability of the cell is increased between about 10% to about 500% as compared to tumor infiltration ability of a cell that does not have the exogenous CD8.

In certain embodiments, the present disclosure provides a method of modifying a cell, the method comprising:
a) introducing into the cell a homologous recombination (HR) template nucleic acid sequence, wherein the HR template comprises:
   i) first and second homology arms homologous to first and second target nucleic acid sequences of a TRAC locus or a TRBC locus;
   ii) a TCR gene sequence positioned between the first and second homology arms; and
   iii) a CD8 gene sequence positioned between the first and the second homology arms;
b) recombining the HR template nucleic acid into a TRAC or TRBC locus; and
c) generating a gene disruption of a TGFβRII locus.

In certain embodiments, the present disclosure provides a method of modifying a cell, the method comprising:
a) introducing into the cell a homologous recombination (HR) template nucleic acid sequence, wherein the HR template comprises:
   i) first and second homology arms homologous to first and second target nucleic acid sequences of a TRAC locus or a TRBC locus; and
   ii) a TCR gene sequence positioned between the first and second homology arms;
b) recombining the HR template nucleic acid into a TRAC or TRBC locus; and
c) generating a gene disruption of a TGFβRII locus.

In certain embodiments of the methods disclosed herein, the CD8 gene sequence encodes a first monomer and a second monomer. In certain embodiments of the methods disclosed herein, each of the first monomer and the second monomer comprise a signal peptide, an extracellular domain, a transmembrane domain, and an intracellular domain. In certain embodiments of the methods disclosed herein,
a) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8α signal peptide, an extracellular domain comprising a CD8α, a transmembrane domain comprising a CD8α, and an intracellular domain comprising a CD8α;
b) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8β signal peptide, an extracellular domain comprising a CD8β, a transmembrane domain comprising a CD8β, and an intracellular domain comprising a CD8β;
c) the first monomer comprises a signal peptide comprising a CD8α signal peptide, an extracellular domain comprising a CD8α, a transmembrane domain comprising a CD8α, and an intracellular domain comprising a CD8β; and the second monomer comprises a signal peptide comprising a CD8α signal peptide, an extracellular domain comprising a CD8α, a transmembrane domain comprising a CD8α, and an intracellular domain comprising a CD8β; or
d) the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

In certain embodiments of the methods disclosed herein,
a) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12;
b) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 13, an extracellular domain comprising an amino acid sequence that is at least about 80% identical to the amino acid sequence set forth in SEQ ID NO: 14, a transmembrane domain comprising an amino acid sequence that is at least about 80% identical to the amino acid sequence set forth in SEQ ID NO: 15, and an intracellular domain comprising an amino acid sequence that is at least about 80% identical to the amino acid sequence set forth in SEQ ID NO: 16;
c) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; or
d) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

In certain embodiments of the methods disclosed herein, the gene disruption of the TGFβRII locus generates a knockout of the TGFβRII gene expression.

In certain embodiments of the methods disclosed herein, the gene disruption of the TGFβRII locus is generated by using a CRISPR/Cas system. In certain embodiments of the methods disclosed herein, the CRISPR/Cas system comprises a gRNA and a Cas9 nuclease.

In certain embodiments of the methods disclosed herein, the gRNA comprises a nucleic acid sequence set forth in SEQ ID NOs: 1-3.

In certain embodiments of the methods disclosed herein, the TCR gene sequence comprises a TCRα gene sequence and a TCRβ gene sequence. In certain embodiments of the methods disclosed herein, TCR gene sequence further comprises a sequence encoding a protease cleavage site, a sequence encoding a 2A peptide, a sequence encoding a signal peptide, or a combination thereof. In certain embodiments of the methods disclosed herein, the HR template further comprises a sequence encoding a protease cleavage site, a sequence encoding a 2A peptide, or a combination thereof. In certain embodiments of the methods disclosed herein, the HR template, from 5′ to 3′, comprises: a) a first sequence encoding a 2A peptide, a sequence encoding an exogenous CD8 receptor, a first sequence encoding a protease cleavage site, a second sequence encoding a 2A peptide, a first sequence encoding a signal peptide, a TCRα gene sequence, a sequence encoding a protease cleavage site, a third sequence encoding a 2A peptide, and a TCRβ gene sequence; or b) a first sequence encoding a 2A peptide, a sequence encoding an exogenous CD8 receptor, a first sequence encoding a protease cleavage site, a second sequence encoding a 2A peptide, a first sequence encoding a signal peptide, a TCRβ gene sequence, a sequence encoding a protease cleavage site, a third sequence encoding a 2A peptide, and a TCRα gene sequence.

In certain embodiments of the methods disclosed herein,
a) the first, second, and third sequences encoding a 2A peptide are codon diverged;
b) the first and second sequences encoding a protease cleavage site are codon diverged; and
c) the first and second sequences encoding a signal peptide are codon diverged.

In certain embodiments of the methods disclosed herein,
a) the first, second, and third 2A peptide comprise the amino acid sequence set forth in SEQ ID NO: 19;
b) the first and second sequences encoding a protease cleavage site comprise the amino acid sequence set forth in SEQ ID NOs: 4-7; and
c) the first and second sequences encoding a signal peptide comprise the amino acid sequence set forth in SEQ ID NO: 35.

In certain embodiments of the methods disclosed herein, the first and second homology arms of the HR template are each from about 300 bases to about 2,000 bases in length.

In certain embodiments of the methods disclosed herein, the method further comprises generating a gene disruption of a TRAC locus. In certain embodiments of the methods disclosed herein, the method further comprises generating a gene disruption of a TRBC locus.

In certain embodiments of the methods disclosed herein, the HR template is non-viral. In certain embodiments of the methods disclosed herein, the HR template is a circular DNA. In certain embodiments of the methods disclosed herein, the HR template is a linear DNA. In certain embodiments of the methods disclosed herein, the introducing occurs via electroporation.

In certain embodiments of the methods disclosed herein, the TCR gene sequence is a patient derived sequence. In certain embodiments of the methods disclosed herein, the TCR gene sequence encodes a TCR recognizing a patient derived cancer antigen. In certain embodiments of the methods disclosed herein, the cancer antigen is a neoantigen. In certain embodiments of the methods disclosed herein, the cancer antigen is a private neoantigen.

In certain embodiments of the methods disclosed herein, the immune cell is a patient-derived primary cell. In certain embodiments of the methods disclosed herein, the immune cell is a T cell, a Natural Killer T cell, a Natural Killer cells, or a lymphocyte. In certain embodiments of the methods disclosed herein, the cell is a T cell.

In certain embodiments of the methods disclosed herein, the method further comprises culturing the cell in the presence of at least one cytokine. In certain embodiments of the methods disclosed herein, the at least one cytokine comprises IL2, IL7, IL15, or a combination thereof. In certain embodiments of the methods disclosed herein, the at least one cytokine comprises IL7 and IL15.

In certain embodiments, the present disclosure provides a cell modified by the methods disclosed herein.

Additionally, in certain embodiments, the present disclosure provides a immune cell comprising an exogenous T cell receptor (TCR) and a dominant negative TGFβRII (dnTGFβRII). In certain embodiments, the present disclosure provides an immune cell comprising an exogenous polynucleotide comprising a sequence encoding an exogenous T cell receptor (TCR) and a sequence encoding a dominant negative TGFβRII (dnTGFβRII), wherein the exogenous polynucleotide is integrated at a TRAC or TRBC locus, wherein the sequence encoding an exogenous TCR is under control of a TRAC or TRBC promoter, and wherein the sequence encoding a dnTGFβRII is under control of an exogenous promoter. In certain embodiments of the cells disclosed herein, the exogenous polynucleotide further comprises an exogenous enhancer, an insulator, a pause element, a Woodchuck Hepatitis Virus Posttranscriptional Regulatory Element (WPRE), a poly-A sequence, or a combination thereof.

Further, in certain embodiments, the present disclosure provides a composition comprising the cells disclosed herein. In certain embodiments of the compositions disclosed herein, In certain embodiments of the compositions disclosed herein, the composition is a pharmaceutical composition comprising a pharmaceutically acceptable excipient. In certain embodiments of the compositions disclosed herein, the composition comprises a crystalloid solution, a cryopreservation agent, serum albumin, or a combination thereof. In certain embodiments of the compositions disclosed herein, the composition comprises Plasma-Lyte A, CryoStor CS10, human serum albumin, or a combination thereof.

In certain embodiments, the present disclosure provides the cell or the composition disclosed herein for use in treating cancer in a subject. In certain embodiments of the cells or compositions for use disclosed herein, the cancer is a solid tumor or a liquid tumor. In certain embodiments of the cells or compositions for use disclosed herein, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV⁺ cancer, esophageal cancer, thyroid cancer, gastric cancer, hepatocellular cancer, cholangiocarcinomas, renal cell cancers, testicular cancer, sarcomas, and colorectal cancer. In certain embodiments of the cells or compositions for use disclosed herein, the liquid tumor is selected from the group consisting of follicular lymphoma, leukemia, and multiple myeloma.

In certain embodiments, the present disclosure provides a method of treating cancer in a subject in need thereof. In certain embodiments of the methods of treating disclosed herein, the method comprises administering a therapeutically effective amount of the cell or the composition disclosed herein. In certain embodiments of the methods of treating disclosed herein, prior to administering the therapeutically effective amount of cells, a non-myeloablative lymphodepletion regimen is administered to the subject. In certain embodiments of the methods of treating disclosed herein, the cancer is a solid tumor. In certain embodiments of the methods of treating disclosed herein, the cancer is liquid tumor. In certain embodiments of the methods of treating disclosed herein, the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments of the methods of treating disclosed herein, the liquid tumor is selected from the group consisting of follicular lymphoma, leukemia, and multiple myeloma.

In certain embodiments, the present disclosure provides a kit comprising the cell, reagents for performing the method, or the composition disclosed herein. In certain embodiments of the kits disclosed herein, the kit further comprises written instructions for treating a cancer.

### EXAMPLES

The following are examples of methods and compositions of the invention. It is understood that various other embodiments may be practiced, given the general description provided above.

### Example 1. Generation of NeoTCR Products

Neoepitope-specific TCRs identified by the imPACT Isolation Technology described in PCT/US2020/17887 (which is herein incorporated by reference in its entirety) were used to generate homologous recombination (HR) DNA templates. These HR templates were transfected into primary human T cells in tandem with site-specific nucleases (see FIGS. 1A-1C). The single-step non-viral precision genome engineering resulted in the seamless replacement of the endogenous TCR with the patient's neoepitope-specific TCR, expressed by the endogenous promoter. The TCR expressed on the surface is entirely native in sequence.

The precision of NeoTCR-T cell genome engineering was evaluated by Targeted Locus Amplification (TLA) for off-target integration hot spots or translocations, and by next generation sequencing based off-target cleavage assays and found to lack evidence of unintended outcomes.

As shown in FIGS. 1A-1C, constructs containing genes of interest were inserted into endogenous loci. This was accomplished with the use of homologous repair templates containing the coding sequence of the gene of interest flanked by left and right HR arms. In addition to the HR arms, the gene of interest was sandwiched between 2A peptides, a protease cleavage site that is upstream of the 2A peptide to remove the 2A peptide from the upstream translated gene of interest, and signal sequences (FIG. 1B). Once integrated into the genome, the gene of interested expression gene cassette was transcribed as single messenger RNA. During the translation of this gene of interest in messenger RNA, the flanking regions were unlinked from the gene of interest by the self-cleaving 2A peptide and the protease cleavage site was cleaved for the removal of the 2A peptide upstream from the translated gene of interest (FIG. 1C). In addition to the 2A peptide and protease cleavage site, a Gly-Ser-Gly (GSG) linker was inserted before each 2A peptide to further enhance the separation of the gene of interest from the other elements in the expression cassette.

It was determined that P2A peptides were superior to other 2A peptides for Cell Products because of their efficient cleavage. Accordingly, two (2) P2A peptides and codon divergence were used to express the gene of interest without introducing any exogenous epitopes from the remaining amino acids on either end of the gene of interest from the P2A peptide. The benefit of the gene edited cell having no exogenous epitopes (i.e., no flanking P2A peptide amino acids on either side of the gene of interest) is that immunogenicity is drastically decreased and there is less likelihood of a patient infused with a Cell Product containing the gene edited cell to have an immune reaction against the gene edited cell.

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to the expression of only TCR sequences encoded by the NeoTCR construct. The general strategy was applied using circular HR templates as well as linear templates.

The target TCRα locus (Ca) is shown along with the plasmid HR template, and the resulting edited sequence and downstream mRNA/protein products in FIGS. 1B and 1C. The target TCRα locus (endogenous TRAC) and its CRISPR Cas9 target site (horizontal stripe, cleavage site designated by arrow) are shown (FIGS. 1A-1C). The circular plasmid HR template with the polynucleotide encoding the NeoTCR is located between left and right homology arms (“LHA” and “RHA” respectively). The region of the TRAC introduced by the HR template that was codon optimized is shown (vertical stripe). The TCRβ constant domain was derived from TRBC2, which is indicated as being functionally equivalent to TRBC1. Other elements in the NeoTCR cassette include: 2A=2A ribosome skipping element (by way of non-limiting example, the 2A peptides used in the cassette are both P2A sequences that are used in combination with codon divergence to eliminate any otherwise occurring non-endogenous epitopes in the translated product); P=protease cleavage site upstream of 2A that removes the 2A tag from the upstream TCRβ protein (by way of non-limiting example the protease cleavage site can be a furin protease cleavage site); SS=signal sequences (by way of non-limited example the protease cleavage site can be a human growth hormone signal sequence). The HR template of the NeoTCR expression gene cassette includes two flanking homology arms to direct insertion into the TCRα genomic locus targeted by the CRISPR Cas9 nuclease RNP with the TCRα guide RNA. These homology arms (LHA and RHA) flank the neoE-specific TCR sequences of the NeoTCR expression gene cassette. While the protease cleavage site used in this example was a furin protease cleavage site, any appropriate protease cleavage site known to one of skill in the art could be used. Similarly, while HGH was the signal sequence chosen for this example, any signal sequence known to one of skill in the art could be selected based on the desired trafficking and used.

Once integrated into the genome (FIG. 1C), the NeoTCR expression gene cassette is transcribed as a single messenger RNA from the endogenous TCRα promoter, which still includes a portion of the endogenous TCRα polypeptide from that individual T cell (FIG. 1C). During ribosomal polypeptide translation of this single NeoTCR messenger RNA, the NeoTCR sequences are unlinked from the endogenous, CRISPR-disrupted TCRα polypeptide by self-cleavage at a P2A peptide (FIG. 1C). The encoded NeoTCRα and NeoTCR polypeptides are also unlinked from each other through cleavage by the endogenous cellular human furin protease and a second self-cleaving P2A sequence motifs included in the NeoTCR expression gene cassette (FIG. 1C). The NeoTCRα and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates the removal of the 2A sequence from the upstream TCRβ chain to reduce potential interference with TCRβ function. The inclusion of a Gly-Ser-Gly linker before each 2A (not shown) further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 (vertical stripe) reduces the likelihood of the entire cassette being lost over time through the removal of intervening sequence of two direct repeats.

In addition to NeoTCR Products, this method can be used for any CD8 Product.

In-Out PCR was used to confirm the precise target integration of the NeoE TCR cassette. Agarose gels show the results of a PCR using primers specific to the integration cassette and site generate products of the expected size only for cells treated with both nuclease and DNA template (KOKI and KOKIKO), demonstrating site-specific and precise integration.

Furthermore, Targeted Locus Amplification (TLA) was used to confirm the specificity of targeted integration. Crosslinking, ligation, and use of primers specific to the NeoTCR insert were used to obtain sequences around the site(s) of integration. The reads mapped to the genome are binned in 10 kb intervals. Significant read depths were obtained only around the intended site the integration site on chromosome 14, showing no evidence of common off-target insertion sites.

Antibody staining for endogenous TCR and peptide-HLA staining for NeoTCR revealed that the engineering results in high frequency knock-in of the NeoTCR, with some TCR− cells and few WT T cells remaining. Knock-in is evidenced by NeoTCR expression in the absence of an exogenous promoter. Engineering was carried out multiple times using the same NeoTCR with similar results. Therefore, efficient and consistent expression of the NeoTCR and knockout of the endogenous TCR in engineered T cells was achieved.

### Example 2. Generation of dnTGFβRII Products

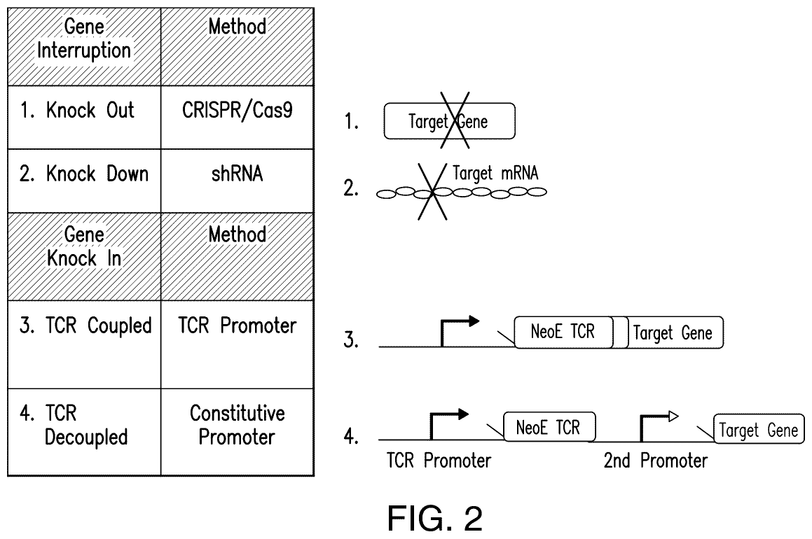

T cell Isolation and Editing. CD4 and CD8 T cells were isolated from healthy donor PBMCs using the Miltenyi Prodigy or Miltenyi MACS separation columns according to the manufacturers' instructions. Positively-selected CD4 and CD8 T cells (using Miltenyi antibodies and isolation column) were used fresh or cryopreserved in 1% human serum albumin (Gemini), 49% plasmalyte (Baxter), and 50% CS10 (Sigma). Cryopreserved cells were thawed, washed in TexMACS (Miltenyi)+10% human AB serum (Valley Biomedical), and seeded at a density of 2×10⁶ cells per mL in culture medium. One day after thaw, or immediately if used fresh, the cells were washed and re-seeded at a density of approximately 1.46×10⁶ cells per mL in culture medium+12.5 ng/mL IL7+12.5 ng/mL IL15+1:17.5 ratio of TransACT T cell activation reagent (or an alternate activation agent) by volume. Two days after activation, T cells were electroporated with a dnTGFβRII Construct plasmid and gRNA-Cas9 RNPs targeting the TCR alpha and beta loci for the production of dnTGFβRII Cells and a dnTGFβRII Product; see, e.g., FIGS. 1A, 1B, 2 (gene knock in schemes 3 and 4), 3B, and 3B. Exemplary expression constructs of the dnTGFβRII Construct are shown in FIGS. 3A and 3B. T cells were electroporated using the Lonza X-unit in 100 μL cuvettes and program EO-115. T cells are expanded in culture medium supplemented with 12.5 ng/mL IL7+12.5 ng/mL IL15. Supplemented medium was exchanged every 2-3 days or as needed until the end of the study, 13 days after activation.

Additional examples of dnTGFβRII Constructs are provided in FIGS. 13A, 13B, 14A, 14B, 15A, 15B, 15C, 16A, 16B, 16C, 17, 18, and 19.

comPACT and comPACT-Dextramer preparation. Neoantigen-specific peptide-HLA complex polypeptides (each a “comPACT”) were prepared according to the method as described in PCT/US2019/025415, hereby incorporated by reference in its entirety. A comPACT-dextramer complex was made for the labeling of NeoTCR expressing T cells. Biotinylated comPACT protein was incubated with a streptavidin-conjugated fluorophore for 10 min at room temperature (RT). Biotin-40-dextran (NANOCS) was added to the mixture and incubated at RT for an additional 10 minutes. The comPACT-Dextramer was stored at 4° C.

Confirmation of comPACT binding to NeoTCR edited T cells. T cells were stained for flow cytometry. Cells were first stained with viability dye for 20 minutes at 4° C., then washed and stained with the comPACT-dextramer for 10 minutes at 4° C. Surface antibodies (anti-CD8α, anti-CD8β, anti-CD4) were added to the suspension of cells and comPACT-dextramer, and the cells are incubated for an additional 20 minutes at 4° C. Cells were then washed and fixed in intracellular fixation buffer (BD Biosciences). All cells were acquired on an Attune NxT Flow Cytometer (ThermoFisher Scientific) and data were analyzed with either FCS Express or FlowJo.

Cytometric Bead Array (CBA). Streptavidin coated plates (Eagle Biosciences) were washed 3 times with wash buffer (PBS supplemented with 1% BSA and 0.05% tween20) and then coated with comPACTs at different concentrations ranging from 1000-0.01 ng/well. Wells with no comPACT and wells coated with mismatched comPACT were used as controls. The plates were incubated for 2 hr at room temperature, washed three times with wash buffer, and then washed three times with TexMACS supplemented with 3% human AB serum to remove the tween20. T cells were given two washes with TexMACS supplemented with 3% human AB serum and resuspended at 1 million cells/mL in TexMACS supplemented with 3% human AB serum and 1× penicillin-streptomycin solution. T cells were plated onto the comPACT coated plate at 100 μL/well and incubated at 37° C., 5% CO2. After 24 h the supernatant was collected, and the cytokine concentrations were analyzed using the BD Cytometric Bead Array (CBA) Human Th1/Th2 Cytokine Kit II (Catalog No. 551809) following the manufacturer's protocol. Capture beads were mixed with culture supernatant, incubated with the detection reagent for 3 hr at RT protected from light, washed, and resuspended in wash buffer. Samples were assayed on an Attune NxT Flow Cytometer and data analyzed with FlowJo. The EC50 represents the concentration of cognate comPACT that elicits 50% of the maximum response and is calculated utilizing a least-squares fit of IFNγ secretion over a range of comPACT concentrations.

Intracellular Staining. T cells were stained for flow cytometry on the indicated days. T cells are first stained with viability dye for 20 minutes at 4° C., then washed and incubated with surface antibodies (anti-CD8α, anti-CD8β, anti-CD4) for an additional 20 minutes at 4° C. T cells are then washed and permeabilized for intracellular staining. T cells are stained with anti-2A peptide or with anti-IFNγ, anti-TNFα, or anti-IL2 in permeabilization buffer for 20 minutes at 4° C. T cells are fixed in intracellular fixation buffer (BD Biosciences). Samples are assayed on an Attune NxT Flow Cytometer (ThermoFisher Scientific) and data analyzed with either FCS Express or Flowlo.

T cell Proliferation Assay. Edited CD4 and CD8 T cells are labeled with the e450 proliferation dye (eBioscience) according to the manufacturer's instructions. Labeled cells were stimulated on comPACT coated plates with a range of concentrations as described above. T cells were harvested over 48-96 hours and analyzed for proliferation as measured by dilution of the e450 dye.

T cell Killing Assay. HLA-matched cell lines were pulsed with the cognate neoantigen peptide or mismatched peptide for 1 h at 37° C., 5% CO2. The cells were washed 3 times with media to remove any unbound peptide and then co-cultured with edited CD4 and CD8 T cells that are labeled with the e450 proliferation dye described above. Co-cultures were incubated for 48 h at 37° C. with 5% CO2 before harvest. Cells were washed and stained with a fixable viability dye to determine killing efficiency. The e450 proliferation dye was used to distinguish edited T cells from target cells.

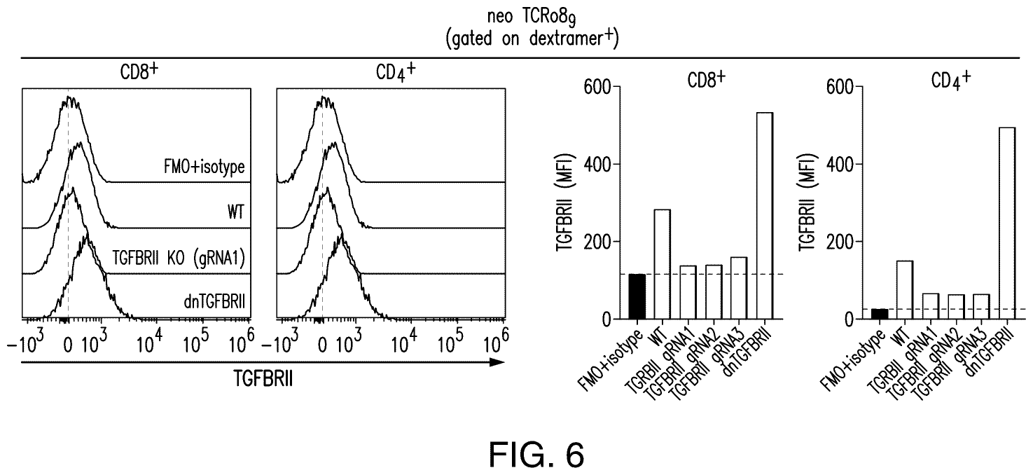

Generation and Validation of an Exemplary dnTGFβRII Product. An expression construct consisting of the coding sequence (CDS) of dnTGFβRII flanked by P2A sites upstream of the NeoTCR beta and alpha sequences was synthesized (see FIG. 3A). Briefly, the dnTGFβRII sequences were synthesized with a GSG-linker and P2A site upstream and flanked with restriction sites (FIG. 3B). The NeoTCR expression vector of interest and the synthesized dnTGFβRII construct were incubated with restriction enzymes and ligated together to create the final HDR construct. The dnTGFβRII Construct was electroporated along with gRNA-Cas9 RNPs targeting the TCRα and β loci. Model NeoTCR (i.e., TCR089) known to bind dextramer was used to demonstrate that expression of the dnTGFβRII transgene did not affect the NeoTCR expression (FIG. 5A). Specifically, this data showed that the expression of the dnTGFβRII gene via the dnTGFβRII Construct described above did not affect the expression of the NeoTCR from the dnTGFβRII Construct. Additional experiments were performed to show that the dnTGFβRII gene was expressed on both CD8+ and CD4+ T cells (FIG. 6).

### Example 3. Generation of TGFβRII Products

T cell Isolation. The isolation of T cells for the manufacture of TGFβRII Products follows the same methods described for the dnTGFβ Products of Example 2.

comPACT and comPACT-Dextramer preparation. The comPACT and comPACT-Dextramer preparation or the manufacture of TGFβRII Products followed the same methods described for the dnTGFβ Products of Example 2.

Confirmation of comPACT binding to NeoTCR edited T cells. The confirmation of comPACT binding to NeoTCR edited T cells follows the same methods described for the dnTGFβ Products of Example 2.

Materials and Methods. The CD8+ and CD4+ T cells isolated as described herein were transfected with gRNAs targeting the TGFβRII as Cas9RNPs along with the TRA and TRB gRNA-Cas9 RNPs as described in Example 1 and in PCT/US2020/17887 (which is herein incorporated by reference in its entirety), resulting in disruption of the endogenous TGFβRII coding sequence and reduced or knocked out TGFβRII protein expression (see FIG. 4).

Specifically, the sgRNAs for the knockout of the TRAC and TRBC were adjusted to a stock concentration of 120 uM in sterile water. Thereafter, the RNPs were complexed at 6:1 sgRNA:Cas9 ratio. The RNP complexes were mixed and incubated at room temperature for 10-20 minutes and then stored on ice or at 4 degrees Celsius. Similar complexing at a ratio of 6:1 was performed for the TGFβRII sgRNAs provided in Table 1. After complexing, each RNP (TRAC, TRBC, and TGFβRII) was combined at a 1:1:1 ratio, plasmid donor DNA (for the introduction of the NeoTCR, see FIGS. 1A-1C) was added, and activated T cells (isolated as described herein) were mixed with the RNPs mixture and electroporated into the activated T cells. FIG. 4 depicts the double knockout of both the TGFβRII and the TCRβ along with the knocking in of the NeoTCR (the neo-TCRβ+TCRα) by disrupting the endogenous TCRα.

Functional studies of the 3 TGFβRII Products made using the TGFβRII sgRNAs provided in Table 1 and the methods described above were performed.

It was shown that the three TGFβRII Products had successful knock down of the endogenous TGFβRII gene expression in the TGFβRII Cells. As shown in FIG. 6, CD8+ and CD4+ TGFβRII Cells (see the TGFβRII gRNA1, TGFβRII gRNA2, and TGFβRII gRNA3 columns and graphs for each of the three TGFβRII Products used in this experiment) were shown to have a significant decrease in TGFβRII expression compared to the wild type CD8+ and CD4+ cells (i.e., cells edited with a NeoTCR knock-in but not electroporated with the TGFβRII sgRNAs).

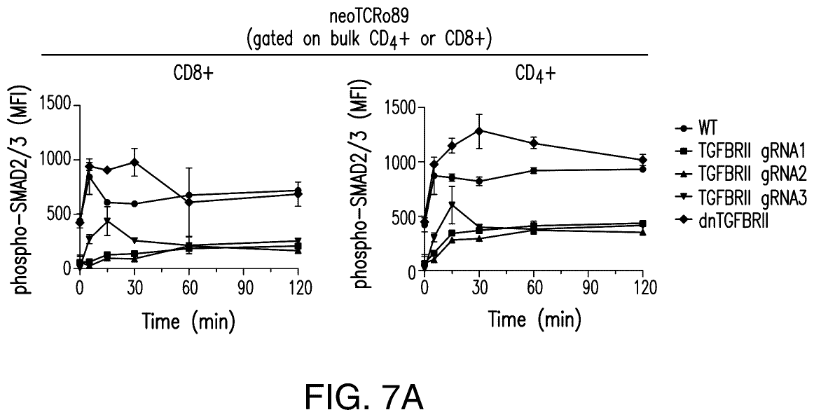

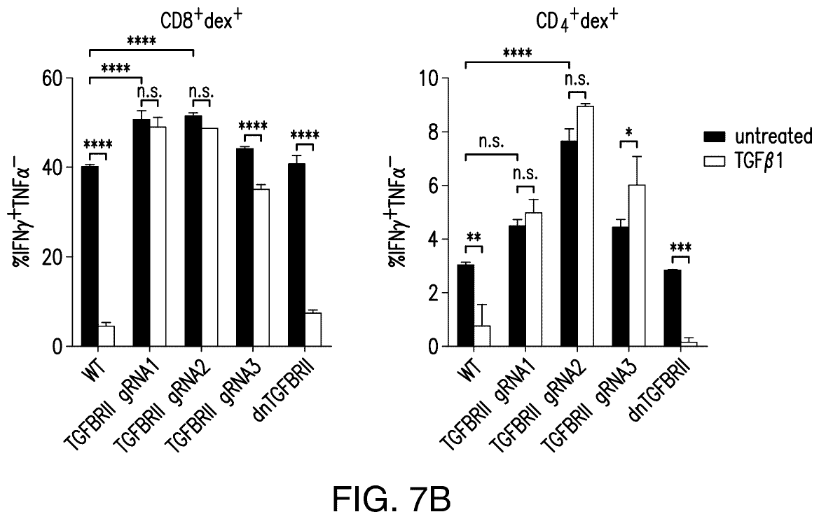

In order to understand the functional significance of the knock down of the TGFβRII gene in TGFβRII Products, the three products were investigated to understand the knock down effect on SMAD2/3 phosphorylation. As shown in FIG. 7A, the knock down of the TGFβRII gene in the TGFβRII Products inhibited TGFβ-induced SMAD2/3 phosphorylation in both CD8+ and CD4+ T cells (see the TGFβRII gRNA1, TGFβRII gRNA2, and TGFβRII gRNA3 columns and graphs for each of the three TGFβRII Products used in this experiment).

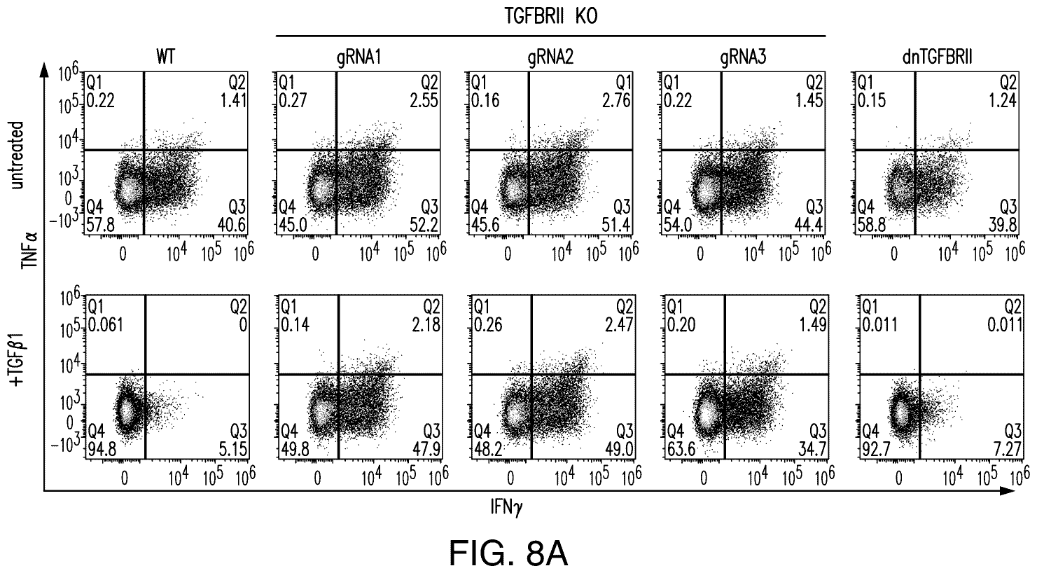

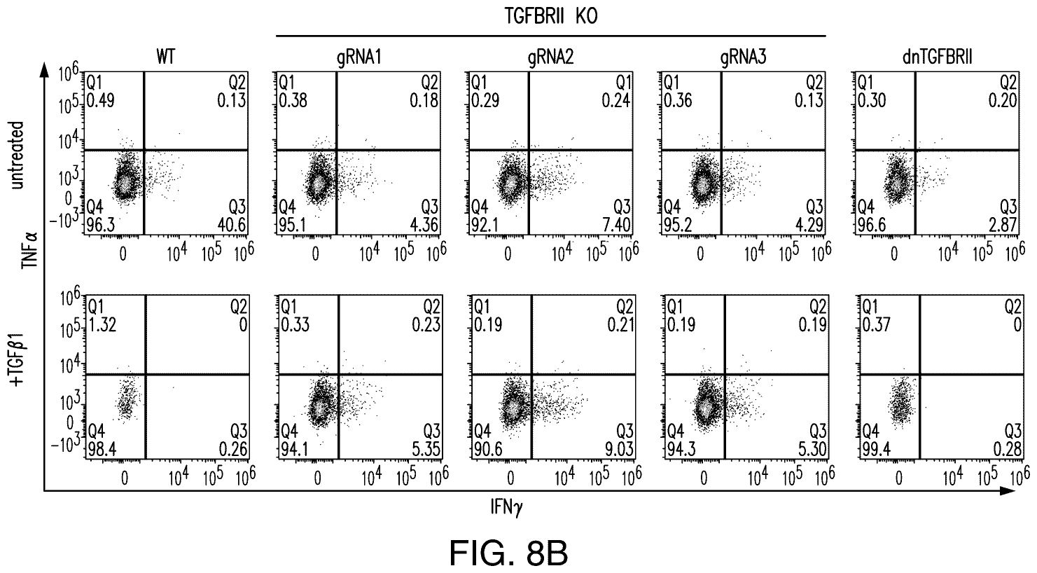

Additional functional studies were carried out in order to understand the effect of TGFβRII knock downs and knockouts on T cell activation and function. FIGS. 7B and 8A-8B show the results of IFNγ experiments. IFNγ upregulates HLA Class 1 and neoantigen presentation on cells. Accordingly, detection of IFNγ is an indicator of T cells functionality and of the TGFβRII Cells detection and killing ability of the neoantigen presenting cells. As shown in FIG. 7B shows that the knock down or knockout of TGFβRII enhances CD8+ and CD4+ T cell IFNγ production in the TGFβRII Cells (see the TGFβRII gRNA1, TGFβRII gRNA2, and TGFβRII gRNA3 columns and graphs for each of the three TGFβRII Products used in this experiment). FIGS. 8A and 8B show FACS plots of the TGFβRII Cells that were gated on Dextramer+ cells (i.e., cells that express the NeoTCR). The FACS experiments show that TGFβ1 inhibits function of the WT NeoTCR Cells (i.e., NeoTCR Cells that do not have a TGFBRII knockdown or knockout) but that the TGFBRII Cells with the TGFβRII knockdown or knockout recovers T cell function as shown by the increase in IFNγ positive cells. The TGFβRII gRNA1, TGFβRII gRNA2, and TGFβRII gRNA3 plots represent each of the three TGFBRII Products made using the sgRNAs provided in Table 1.

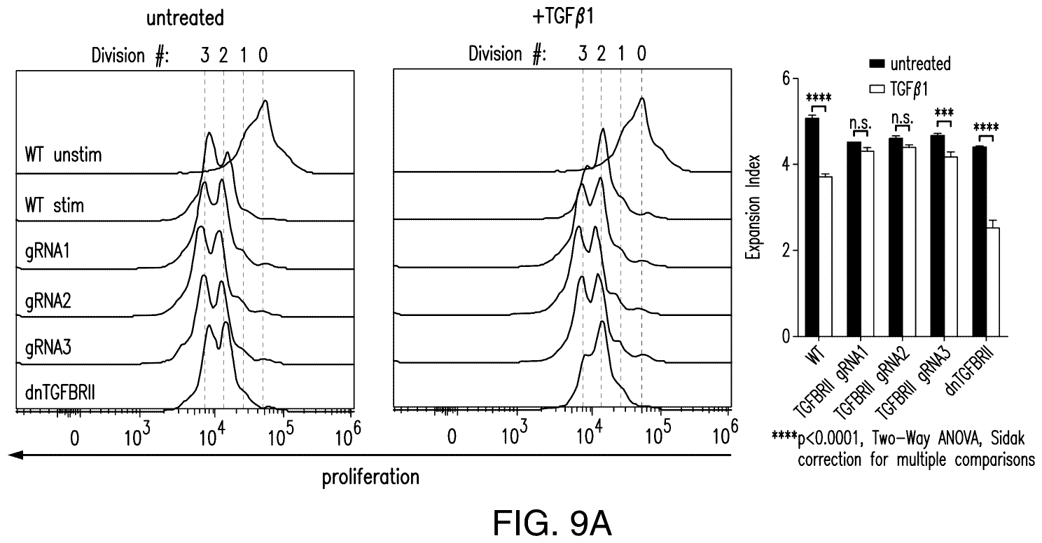

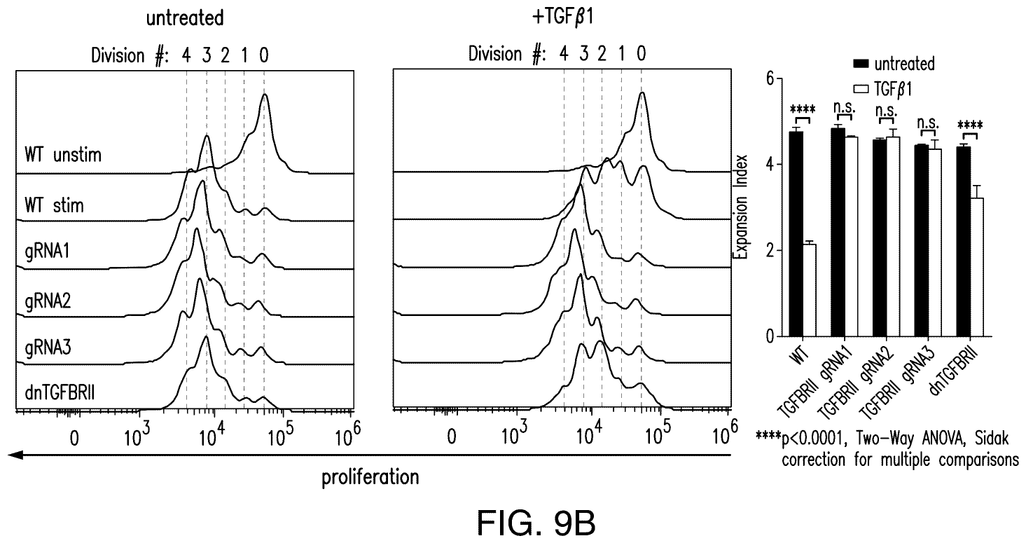

T cell proliferative capacity of the TGFβRII Products was also examined. FIGS. 9A and 9B show that the knockout or knockdown of TGFBRII restores T cell proliferation in the presence of TGFβ1 in CD8+ and CD4+ cells.

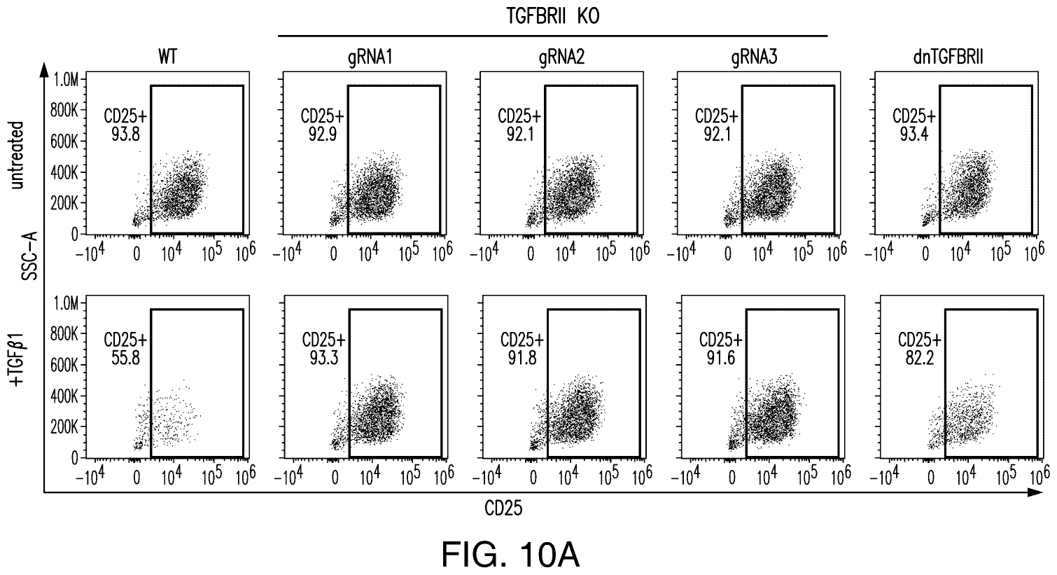

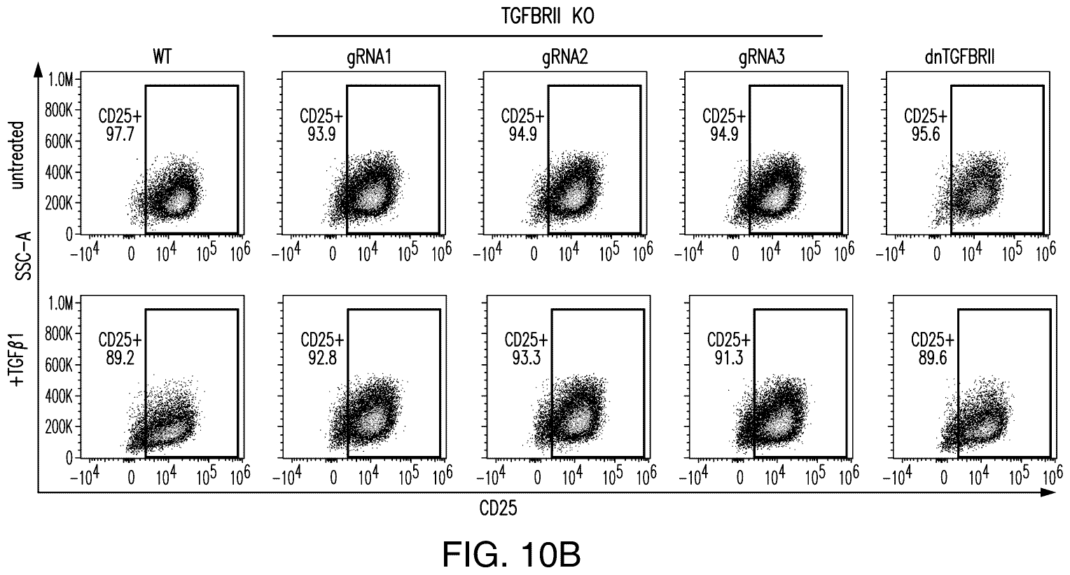

T cell activation capacity was examined using the SSC-A and CD25 markers. SSC-A detects how granular a cell is wherein the more granular a cell is the more activated it is. CD25 (i.e., the IL2Rα chain) is an activation marker such that the expression of CD25 increases when a T cell is activated. As shown in FIGS. 10A and 10B, the three TGFβRII Products (TGFβRII gRNA1, TGFBRII gRNA2, and TGFBRII gRNA3) present with increased granularity (SSC-A) and increased CD25 expression indicating that the TGFBRII Products are able to activate in the presence of TGFβI exposure whereas the WT NeoTCR Cells (i.e., NeoTCR Cells that do not have a TGFβRII knockdown or knockout) have limited activity compared to the TGFBRII Cells. The FACS plots of the TGFBRII Cells in these experiments were gated on Dextramer+ cells (i.e., cells that express the NeoTCR).

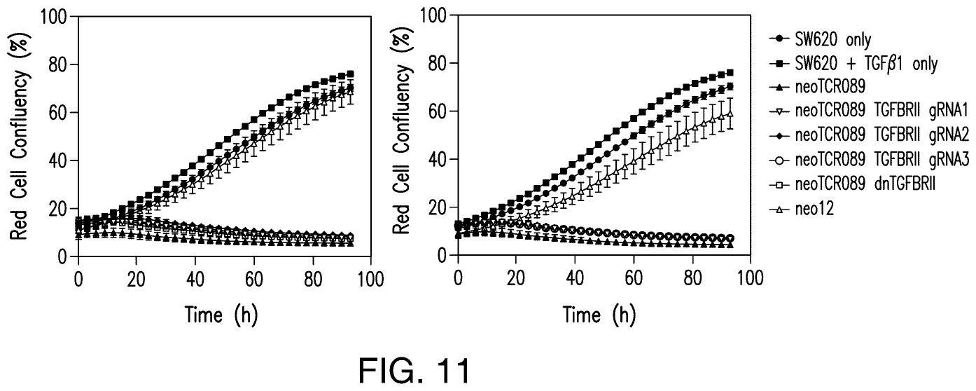

Lastly, given that the TGFBRII Products were designed to treat cancers and other proliferative disorders, killing assays were performed to assess the ability of the TGFBRII Product to kill cells that express a cognate neoantigen. FIG. 11 shows the data from a killing assay with SW620 cells that were engineered to express the cognate antigen to the NeoTCR Cells of the experiment (in this experiment, the NeoTCR used was the NeoTCR089 and the antigen used is the cognate antigen that is specific to the NeoTCR089). The data from a E:T ratio of 2:1 is shown (1:1, 1:2, and 1:4 ratios were also used and exhibited similar results, data not shown). As shown, SW620 killing was not significantly affected by the addition of TGFβ1 (pre-incubation for 2 hours and added to the cell culture).

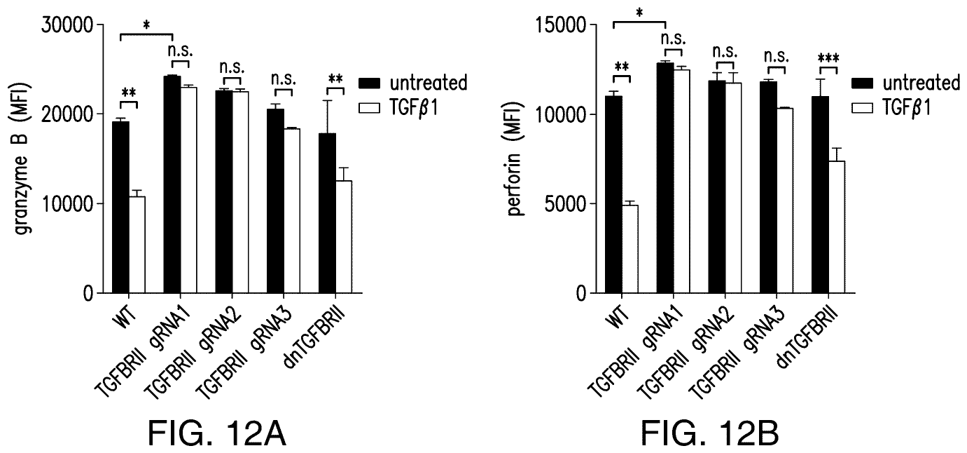

Furthermore, perforin and granzyme B experiments were performed to further assess the killing ability of the TGFβRII Products. Perforin and granzyme B were used as killing ability markers because perforin is a molecule that perforates cells and granzyme B is the execution molecule that initiates apoptosis of the perforated cells. Accordingly, increased expression of both perforin and granzyme B indicates an increased killing capacity. As shown in FIGS. 12A and 12B, TGFβ1 reduces the frequency of granzyme B+ perforin+CD8+ cells; however, such reduction is greatly reduced in the TGFBRII Products indicating that the TGFβRII Products have an increased killing capacity compared to the WT NeoTCR Cells (i.e., NeoTCR Cells that do not have a TGFBRII knockdown or knockout).

### Example 3. Generation of CD8-TGFBRII Products

T cell Isolation. The isolation of T cells for the manufacture of TGFβRII Products will follow the same methods described for the dnTGFβ Products of Example 2.

comPACT and comPACT-Dextramer preparation. The comPACT and comPACT-Dextramer preparation or the manufacture of TGFβRII Products will follow the same methods described for the dnTGFβ Products of Example 2.

Confirmation of comPACT binding to NeoTCR edited T cells. The confirmation of comPACT binding to NeoTCR edited T cells will follow the same methods described for the dnTGFβ Products of Example 2.

Materials and Methods for the knockdown and knockout of the TGFβRII gene. The methods for knocking down and knocking out the TGFRβII gene will be the same methods used and described in Example 3.

Materials and Methods used for the knock in of the CD8 Constructs. As described herein, four CD8 Products were generated:
1. CD8α homodimer (CD8 Construct 1)
2. CD8α-P2A-CD8β (CD8 Construct 2)
3. CD8α with CD8β intracellular domain (CD8 Construct 3)
4. CD8α homodimer with CD4 intracellular domain (CD8 Construct 4)

As shown in FIGS. 20A-20D, 21A-21D, 22A, and 22B, these CD8 Constructs were designed to allow for varying degrees of LCK affinity. As predicted, CD8 Product 1 was shown to have the lowest LCK affinity, followed by CD8 Product 2, CD8 Product 3, and CD8 Product 4 (in that order with CD8 Product 4 having the highest LCK affinity.

Based on the high affinity of CD8 Construct 4, this product was used in cell killing assays to exemplify the increased cell killing ability of CD8 Products 1-4 compared to NeoTCR Products.

The methods for knocking in the dnTGFβRII and knocking in of the NeoTCR described in Example 2, can be used for the CD8-TGFβRII Cells and Products with the exception of the plasmids encoding the dnTGFβRII and NeoTCR are replaced with the plasmids encoding an engineered CD8 molecule and a NeoTCR (see the constructs provided in FIGS. 20A-20D, 21A-21D, 22A, and 22B). As described in Example 3, the plasmid DNA used for the knock in (the CD8 constructs) can be combined with the sgRNAs for the knockdown or knockout of the TGFβRII gene in a single electroporation reaction in order to make CD8-TGFβRII Cells and a CD8-TGFβRII Product.

### Example 4. Generation of CD8-TGFBRII Products

Cell labeling and culture for proliferation assay. 10⁶ total T cells were harvested from each sample of two different donors and washed twice in 1×PBS (without Ca²⁺ or Mg²⁺). Cells were then resuspended in PBS and mixed 1:1 with 20 M e450 proliferation dye (Thermo Fisher Scientific), and incubated at 37° C. for 10 min to label cells. The labeling reaction was quenched by adding RPMI1640 containing 10% FBS and cells were washed twice with TexMACS media (Miltenyi Biotec) containing 3% human AB serum (Valley Biomedical). Cells were resuspended in TexMACS media containing 3% human AB serum and 2 ng/mL IL-7 and 2 ng/mL IL-15 (both from Miltenyi Biotec), and with or without 1 ng/mL of recombinant human TGFβ1 (R&D Systems). Cells were plated at 10⁶ cells/well in 200 μL in each well of neoE-HLA-coated, 96 well plates (Eagle Biosciences). Cells were incubated at 37° C. and 5% CO2 and harvested 3 days later and analyzed by flow cytometry.

Cell staining for proliferation assay and analysis. Cells were first stained for 15 min at 4° C. with dextramer for the cognate NeoTCR, antibodies for CD4 (SK3; Biolegend), CD8a (Hit8a; Biolegend), CD25 (4E3; Thermo Fisher Scientific), and eBioscience Fixable Viability Dye eFluor 780 (Thermo Fisher Scientific). Cells were washed and fixed for 20 min at 4° C. using the eBioscience Intracellular Fixation & Permeabilization Buffer Set (Thermo Fisher Scientific). Intracellular staining for 2A peptide (NovusBio) was then performed for 20 min at 4° C., and cells were washed twice with 1× Permeabilization/Wash buffer and then resuspended in BSA Stain Buffer (BD Biosciences) and analyzed by flow cytometry on a Thermo Fisher Attune and FlowJo 10.8.0 (BD). Expansion index, which is the fold expansion of the overall culture, was determined using the Proliferation Modeling feature in FlowJo. Cells were gated on lymphocytes, singlets, live and then on gene-edited (2A+) cells, and CD4+ and CD8+ cells were then analyzed separately.

Cell culture for intracellular cytokine staining. Cells were resuspended in TexMACS media (Miltenyi Biotec) containing 3% human AB serum (Valley Biomedical) and 2 ng/mL each of IL-7 and IL-15 (both from Miltenyi Biotec), and with or without 1 ng/mL of recombinant, human TGFβ1 (R&D Systems), and then plated at 100,000 cells/well in 200 μL in each well of neoE-HLA-coated, 96 well plates (Eagle Biosciences). Cells were incubated for 3 days at 37° C. and 5% CO2. During the last 4 h of stimulation, 50 μL of supernatant was removed and brefeldin A (Thermo Fisher Scientific) in TexMACS media with 3% human AB serum was added at a 1× final concentration.

Surface and intracellular cytokine staining. Cells were harvested and washed with BD Stain Buffer (BD Biosciences). Cells were then stained for 15 min at 4° C. with antibodies for CD4 (SK3; Biolegend), CD8α (Hit8a; Biolegend), 41BB (4B4-1; Biolegend), OX40 (ACT34; Biolegend), CD40L (TRAP1; BD Biosciences), and Fixable Near-IR dead cell stain (Thermo Fisher Scientific). Cells were washed and fixed for 20 min at 4° C. using the Cytofix/Cytoperm solution (BD Biosciences). Intracellular staining for IFNγ (B27; BD Biosciences) and TNFα (MAb11; Biolegend) was then performed for 30 min at 4° C. Cells were washed twice with 1× Permeabilization/Wash buffer and then fixed in IC fix buffer (Thermo Fisher Scientific). Cells were resuspended in BSA Stain Buffer (BD Biosciences) and analyzed by flow cytometry on a Thermo Fisher Attune and FlowJo 10.8.0 (BD).

Cell culture for activation marker staining. Cells were resuspended in TexMACS media (Miltenyi Biotec) containing 3% human AB serum (Valley Biomedical) and 2 ng/mL each of IL-7 and IL-15 (both from Miltenyi Biotec), and with or without 1 ng/mL of recombinant, human TGFβ1 (R&D Systems), and then plated at 100,000 cells/well in 200 μL in each well of neoE-HLA-coated, 96 well plates (Eagle Biosciences). Cells were incubated for 3 days at 37° C. and 5% CO2.

Surface staining. Cells were harvested and washed with BD Stain Buffer (BD Biosciences). Cells were then stained for 15 min at 4° C. with dextramer for the cognate NeoTCR, antibodies for CD4 (SK3; Biolegend), CD8a (Hit8a; Biolegend), 41BB (4B4-1; Biolegend), OX40 (ACT34; Biolegend), CD40L (TRAP1; BD Biosciences), and Fixable Near-IR dead cell stain (Thermo Fisher Scientific). Cells were washed and fixed in IC fix buffer (Thermo Fisher Scientific). Cells were resuspended in BSA Stain Buffer (BD Biosciences) and analyzed by flow cytometry on a Thermo Fisher Attune and FlowJo 10.8.0 (BD).

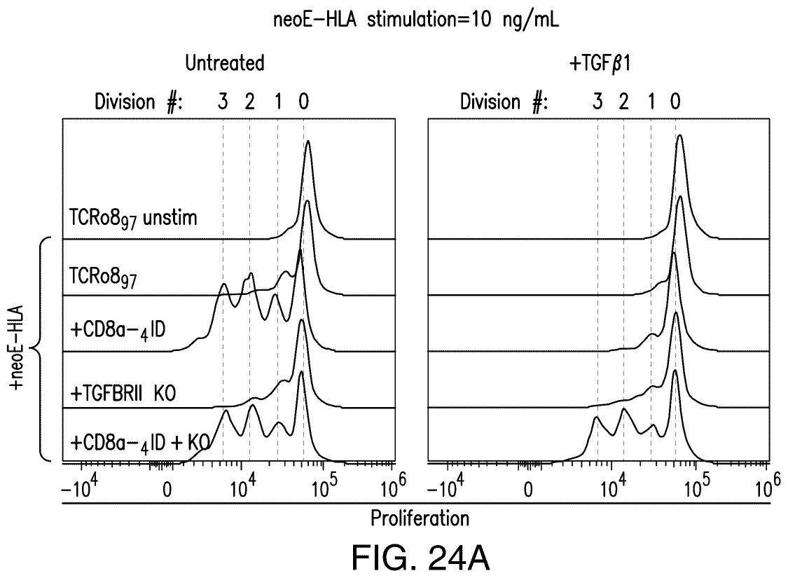

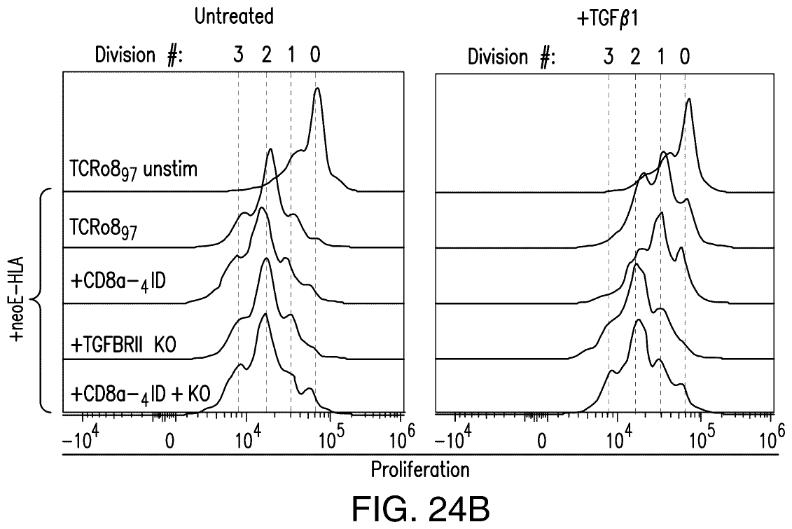

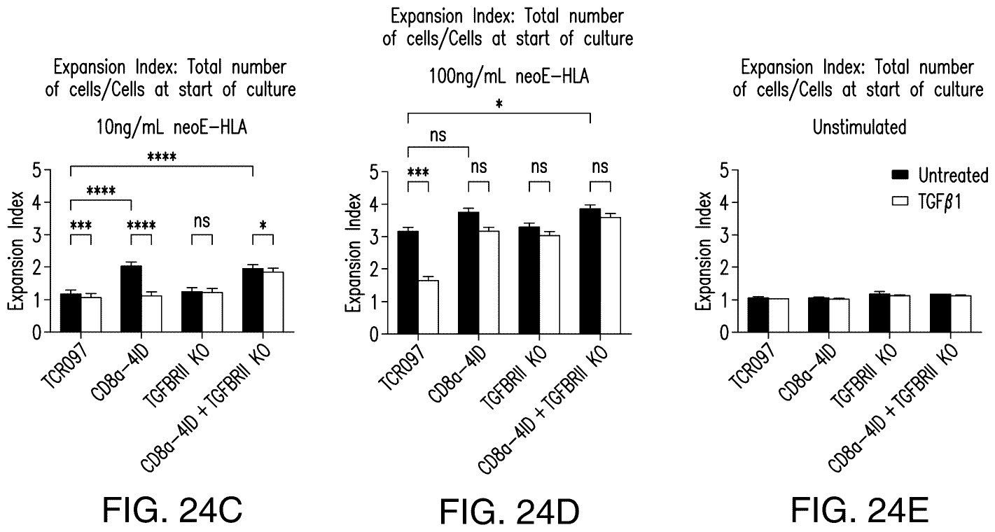

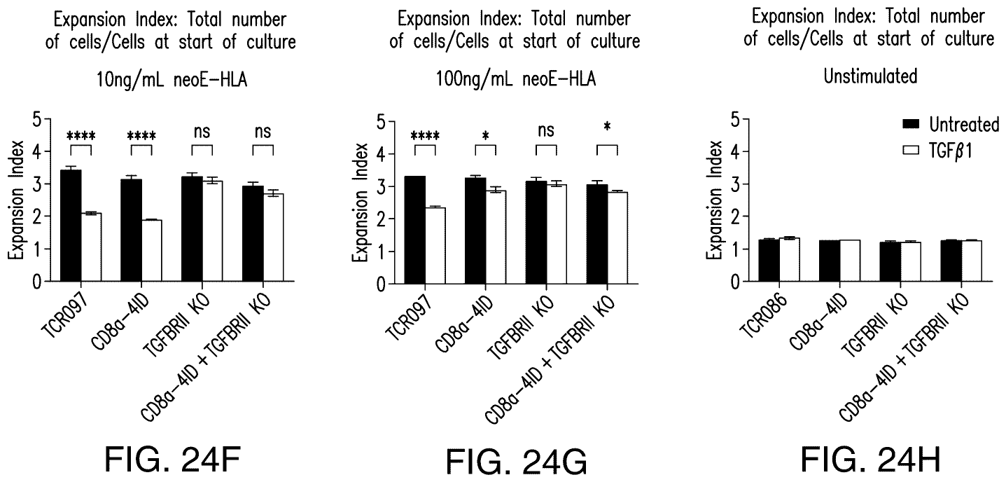

Results: Proliferation and Activation. As shown in FIGS. 24A-24H, overexpression of exogenous CD8 was sufficient to enhance proliferation of CD4 and CD8 T cells in presence of low antigen stimulation (10 ng/ml of neoE-HLA), independently of the knockout of TGFBRII. Since the tumor microenvironment is enriched in immunosuppressive cytokines such as TGFβ, it was determined whether the knockout of TGFBRII would be sufficient to protect from its immunosuppressive effects. Notably, knockout of TGFBRII improved the proliferation rate of CD4 and CD8 T cells at low and high antigen stimulation (10 ng/ml and 100 ng/ml, respectively). These data demonstrate that T cells expressing an exogenous CD8 and knockout for TGFBRII can proliferate at low antigen stimulation levels and are resistant to immunosuppressive stimulate (e.g., TGFβ).

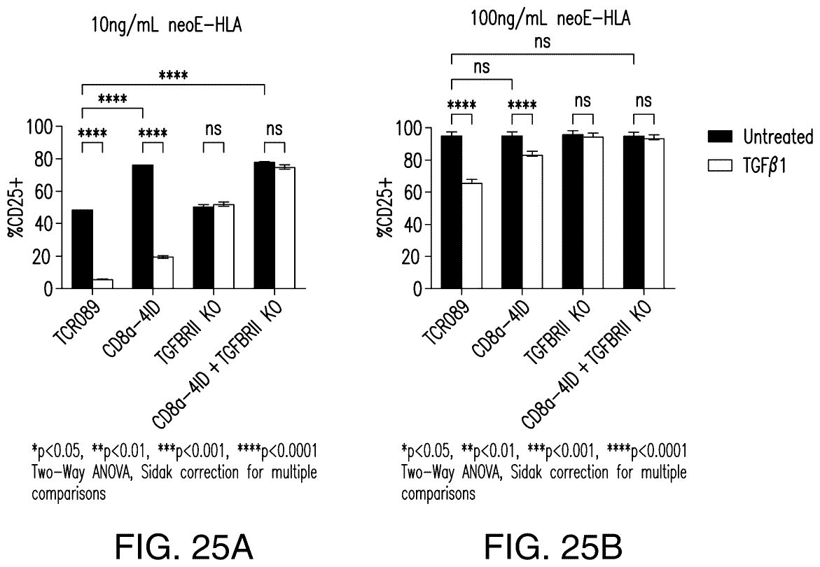

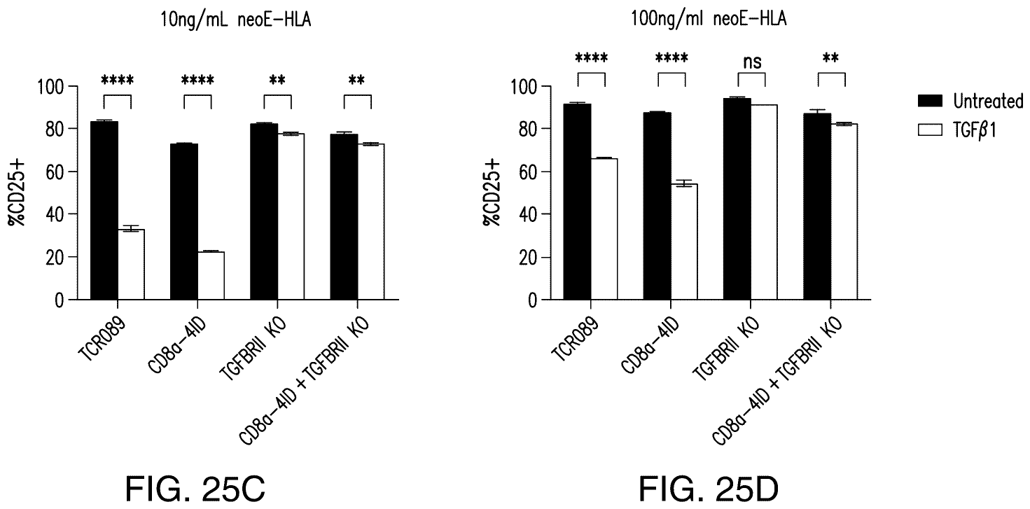

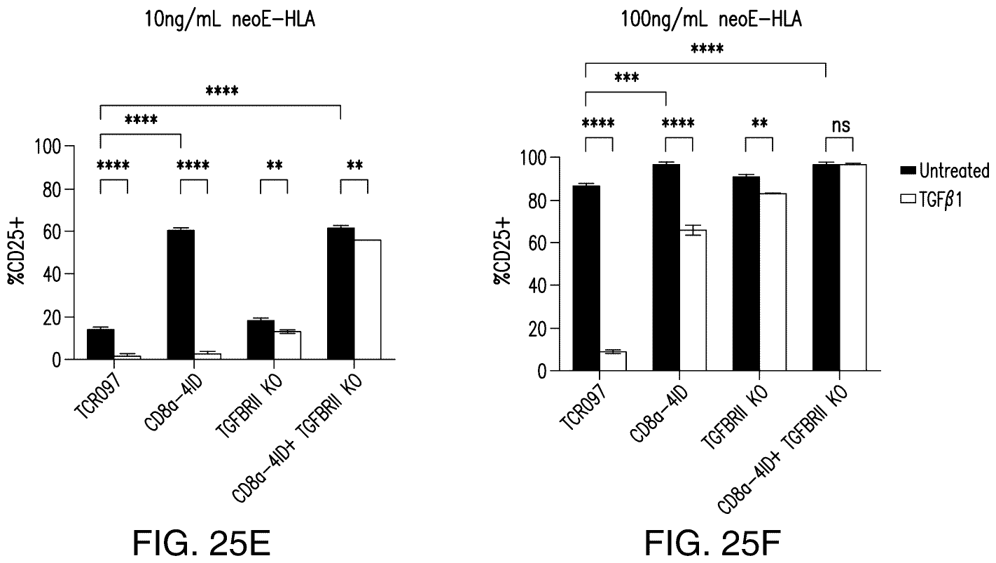

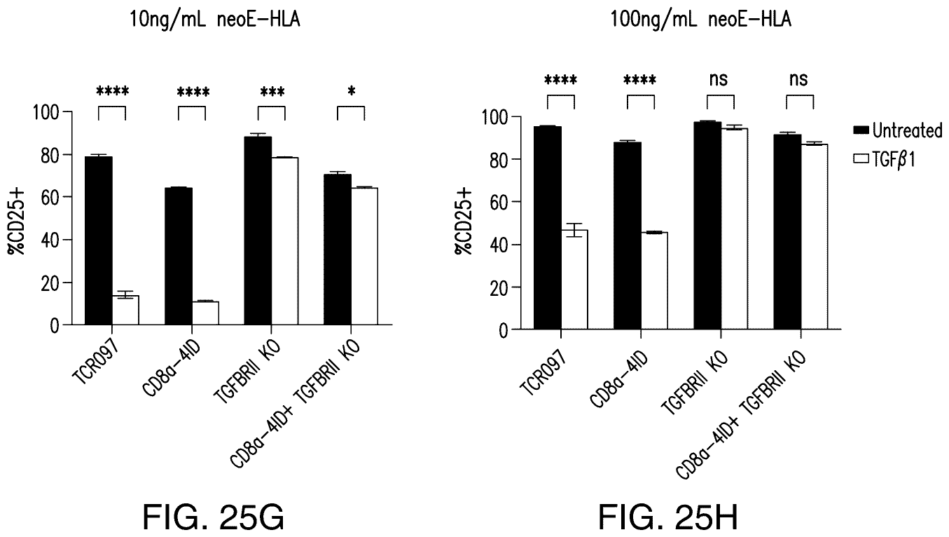

Next, it was determined whether the presently disclosed cells were able to show improved activation levels. For this purpose, the expression levels of CD25 were analyzed in different experimental conditions. CD4 and CD8 T cells expressing exogenous CD8 and knockout for TGFBRII showed significantly increased activation levels and were resistant to the effects of TGFβ (a cytokine usually found in the milieu of the tumor microenvironment). See FIGS. 25A-25H.

These data show that knockout of TGFBRII rescues the anti-proliferative effects of TGFβ in both CD4 and CD8 T cells upon low antigen stimulation (10 ng/ml neoE-HLA); furthermore, the knockout of TGFBRII can rescue the effects of TGFβ on CD25 expression to levels similar to untreated cells. Also, in the absence of TGFβ, the expression of exogenous CD8 enhances the proliferation of CD4 T cells, independently of the TGFBRII knockout. Collectively, these data support the concept that beneficial effects of exogenous CD8 and knockout of TGFBRII are maintained when these modifications were made at the same time.

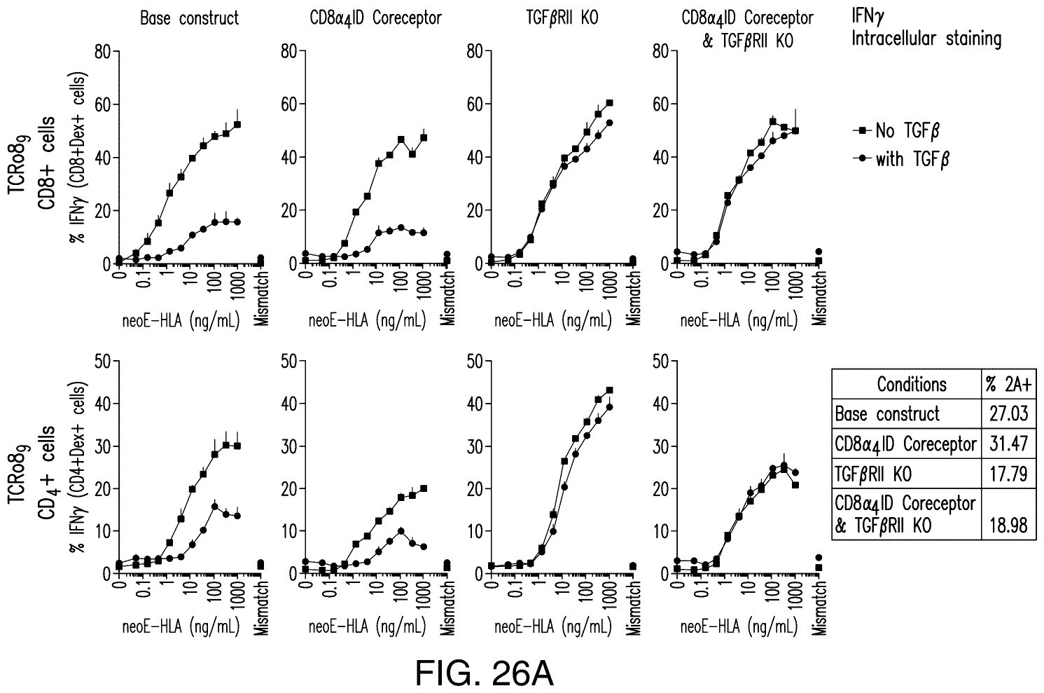

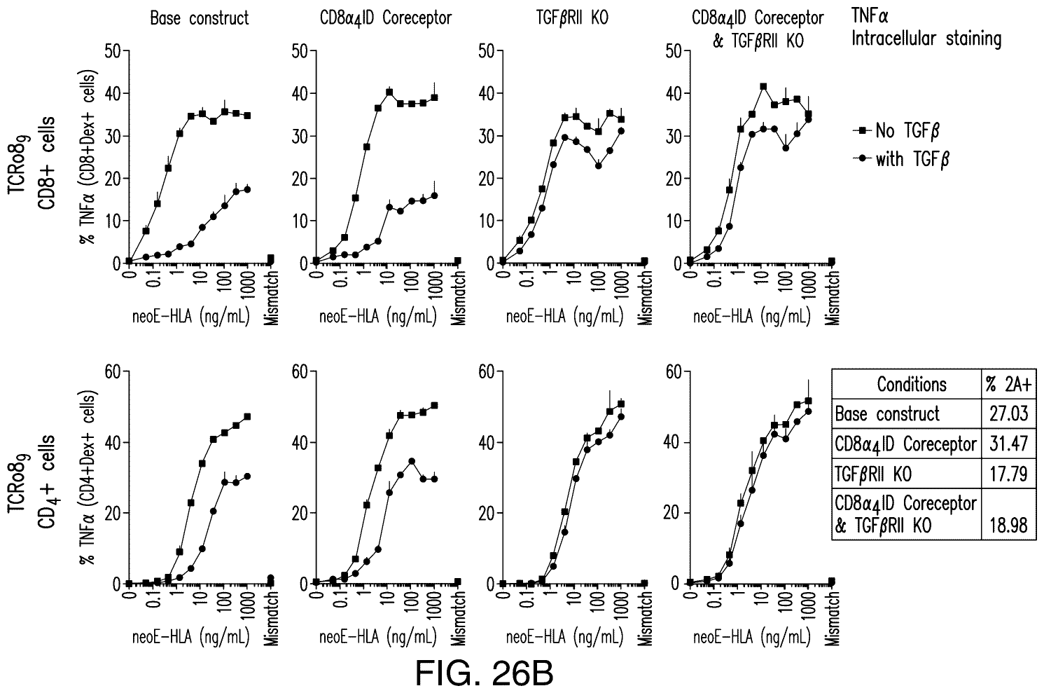

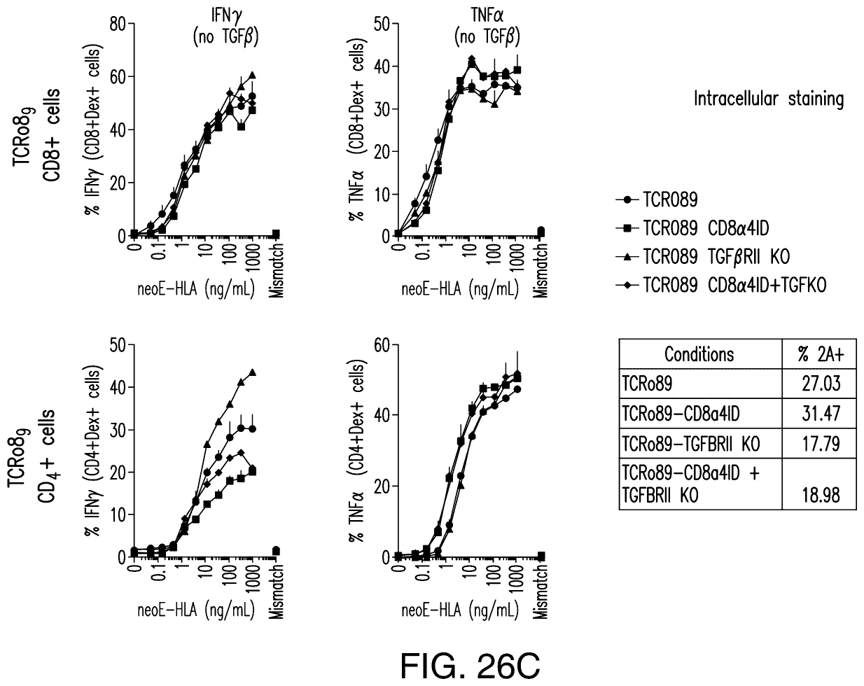

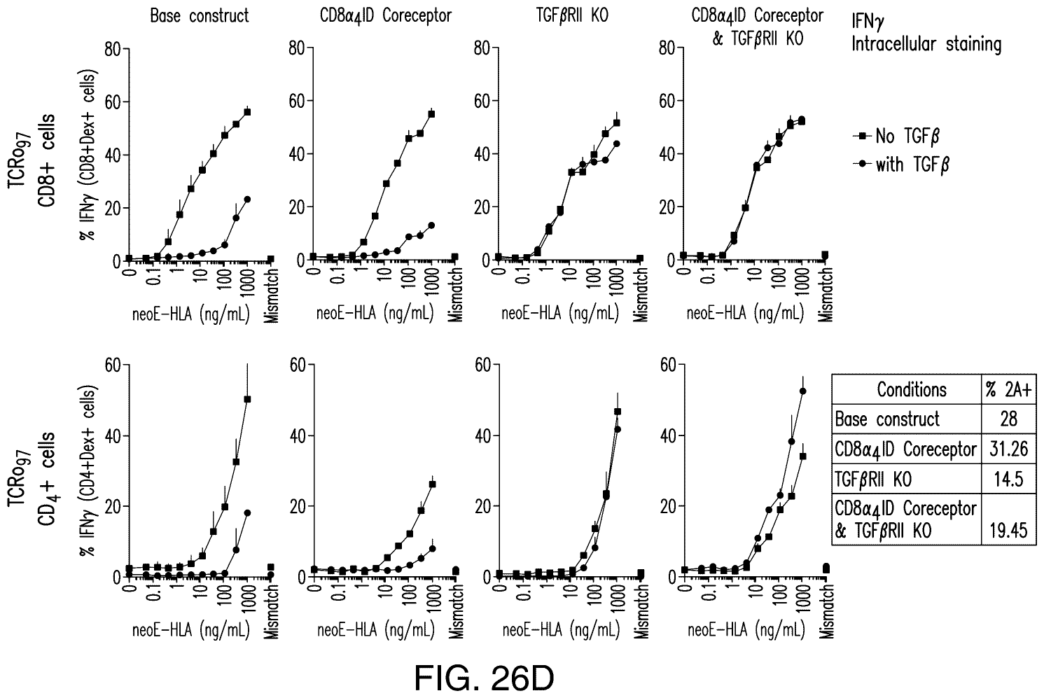

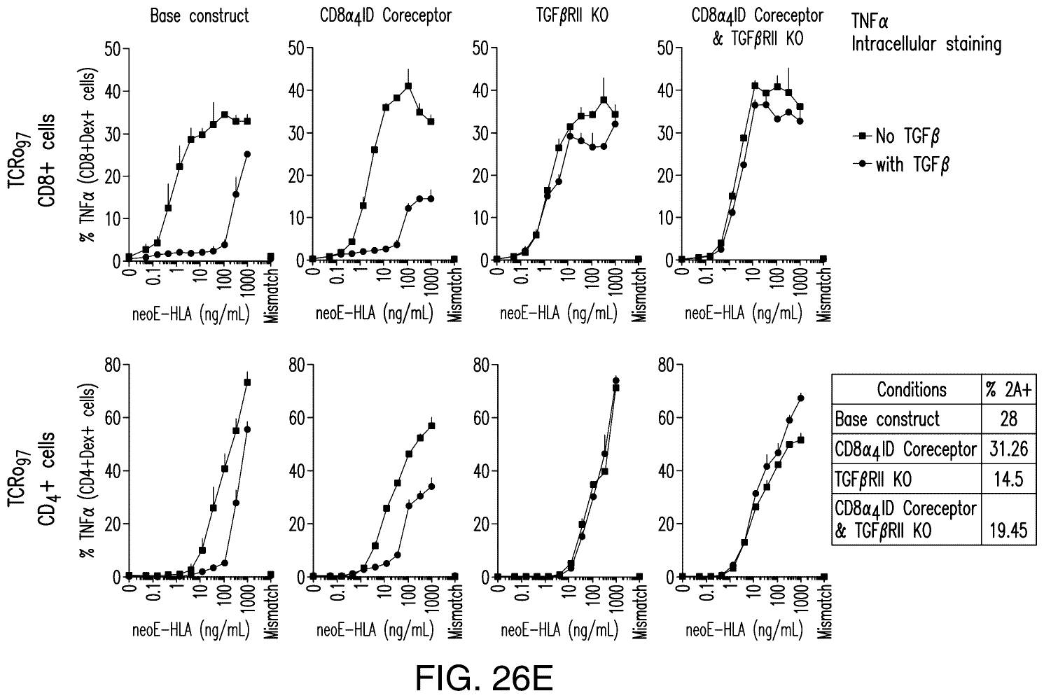

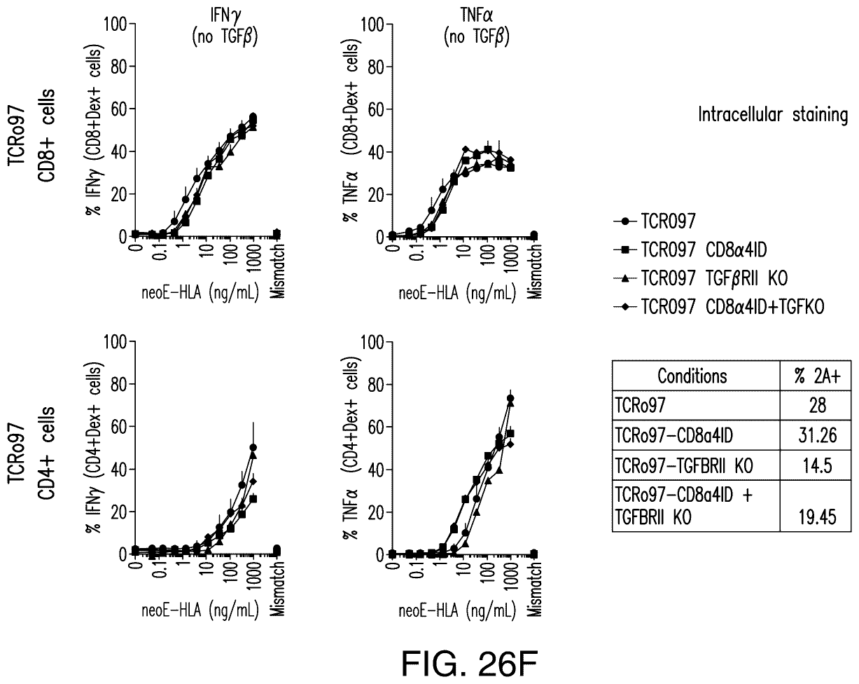

Results: Intracellular Cytokines and Activation Markers. Next, it was verified whether exogenous CD8 and knockout of TGFBRII could have an effect on the intracellular cytokine expression and certain activation markers. As shown in FIGS. 26A-26F, TGFBRII knockout rescued CD4 and CD8 T cells from the inhibitory effects of TGFβ, for example, by maintaining the expression of IFNγ and TNFα cytokines. Notably, while the knockout of TGFBRII rescued the T cells from the immunosuppressive effects of TGFβ, exogenous CD8 improved TNFα expression in CD4 T cells.

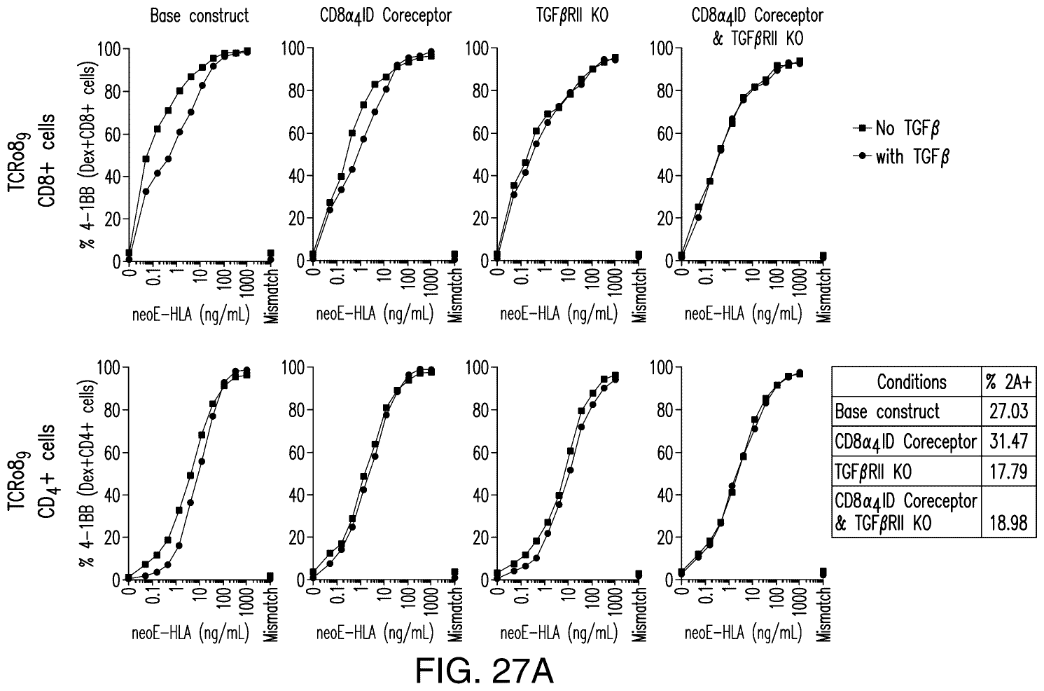

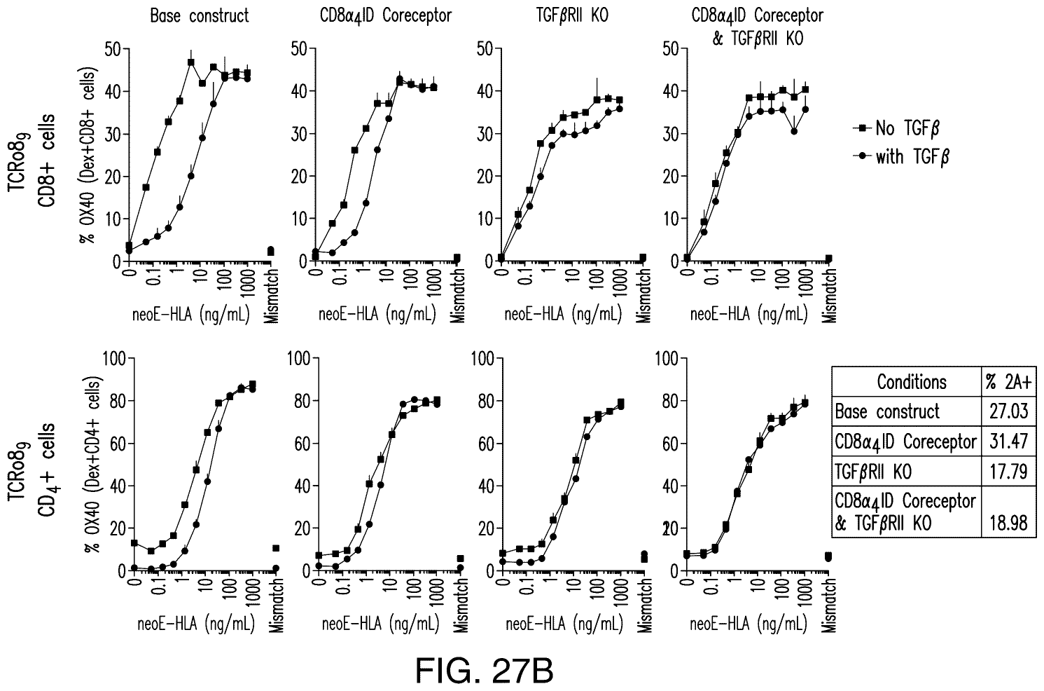

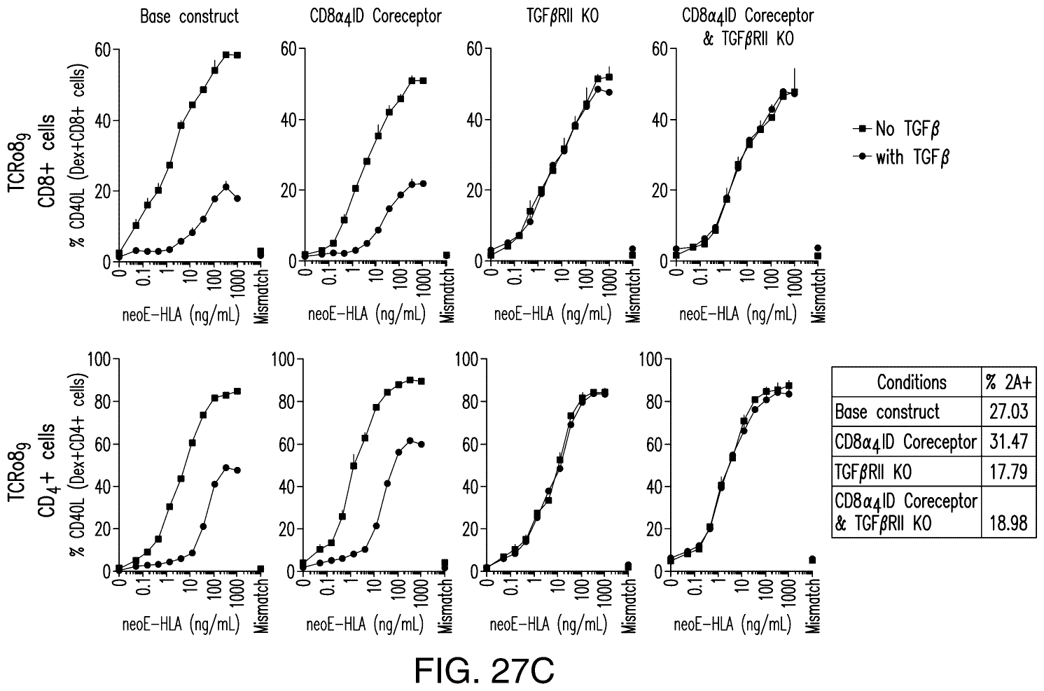

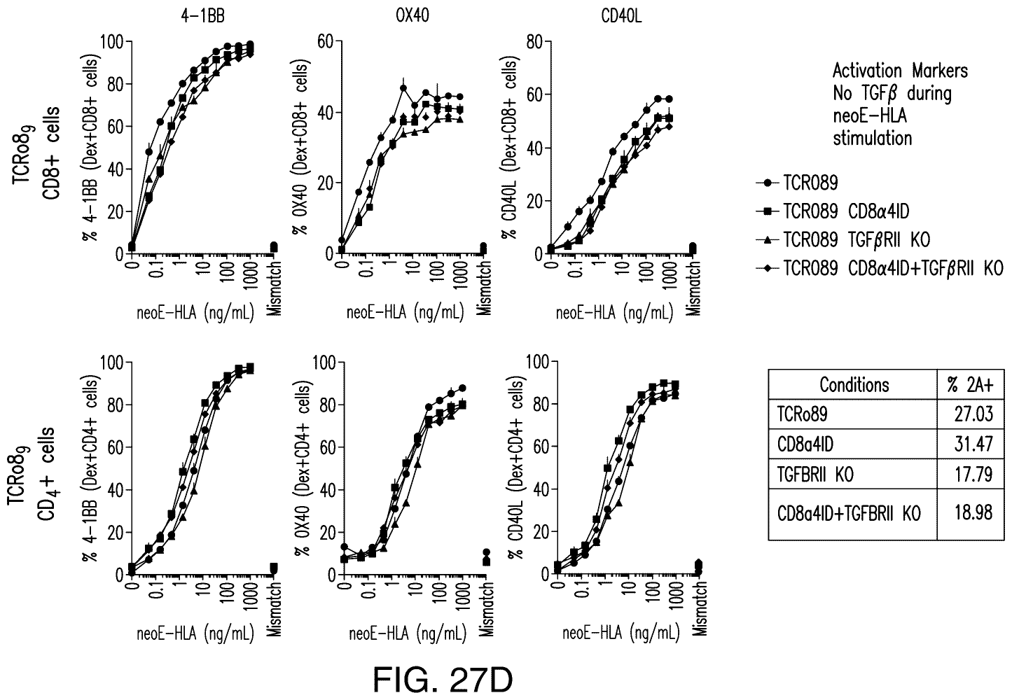

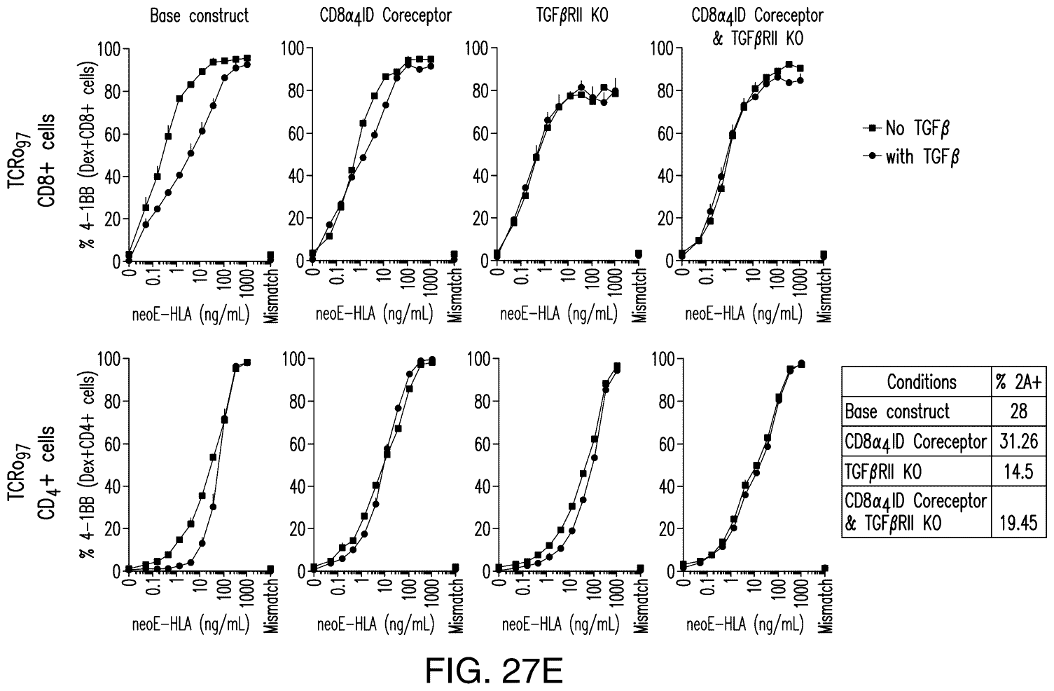

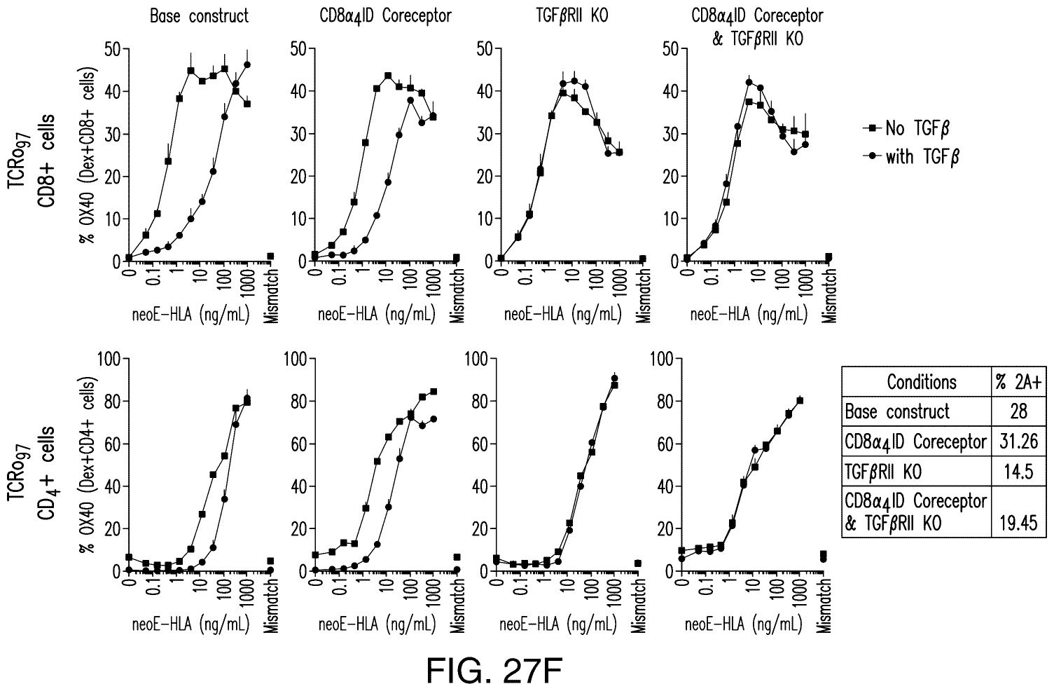

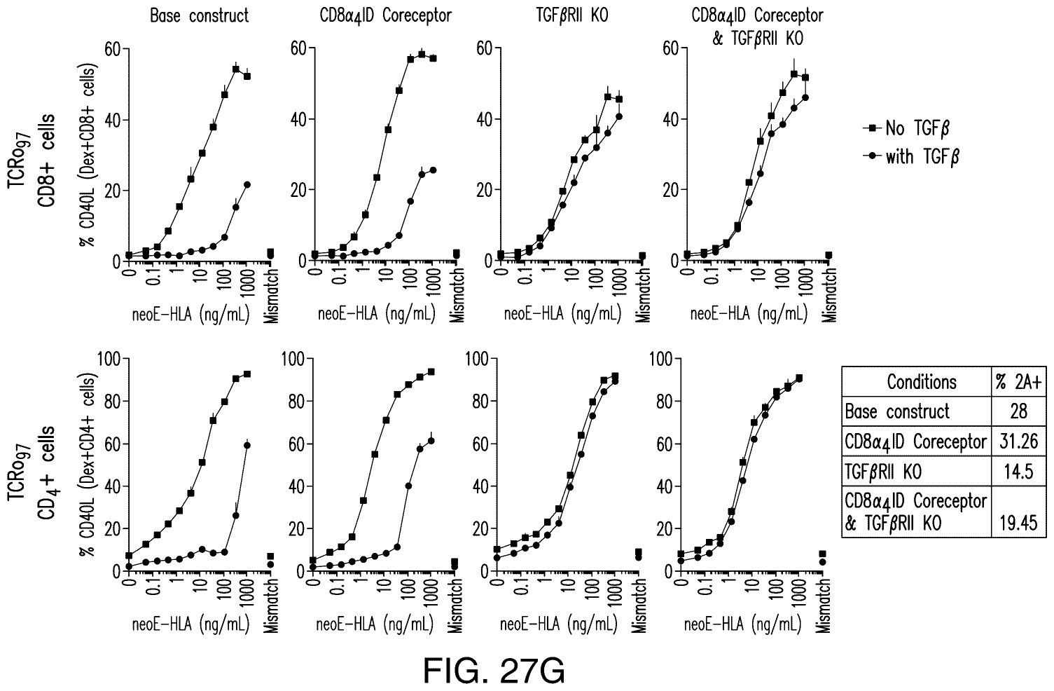

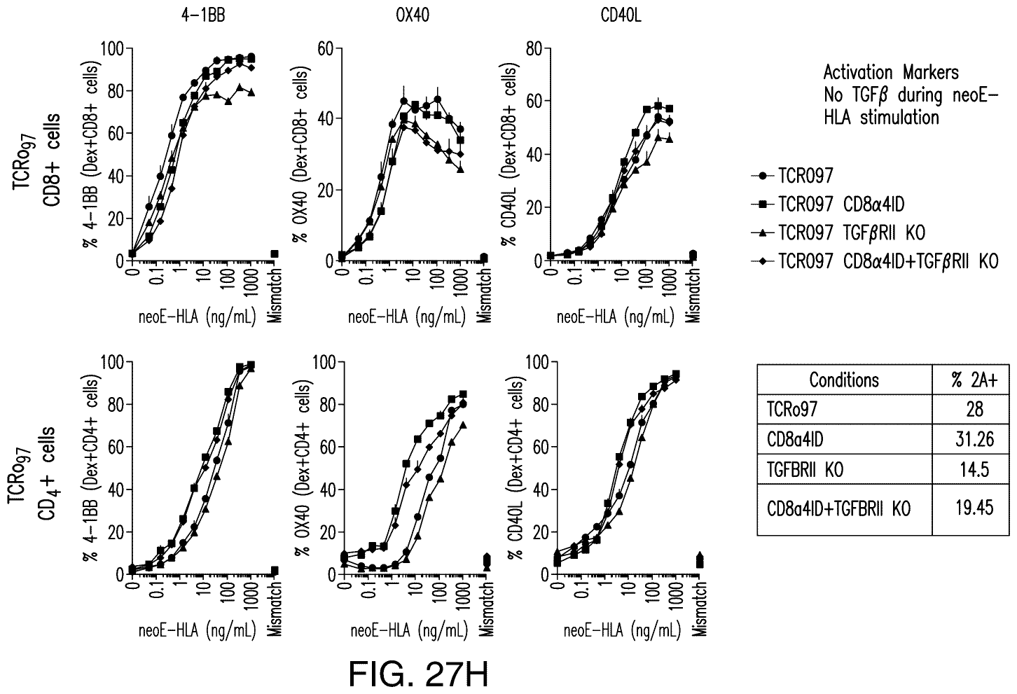

Finally, it was verified whether the presently disclosed cells had improved the expression profile of certain immunological markers such as 4-1BB, OX40, and CD40L. As depicted in FIGS. 27A-27H, knockout of TGFBRII prevented immunosuppressive effects of TGFβ in both CD4 and CD8 T cells. For example, levels of OX40 and CD40L were not impaired by incubating the cells with TGFβ. Further, the addition of exogenous CD8 resulted in improved expression of OX40 in CD4 T cells (see FIG. 27F). These data demonstrate the beneficial effects of the knockout of TGFBRII on the activation of the T cells and the synergistic effect with the exogenous CD8 for recognition of low-affinity antigens.

### Example 5. Generation of CD8-TGFBRII Products

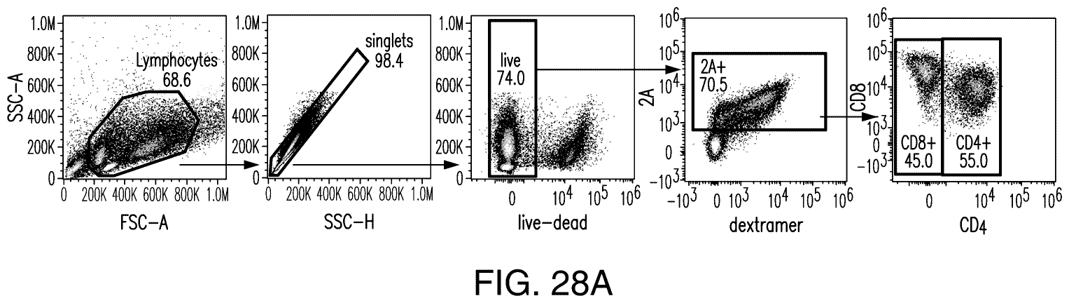

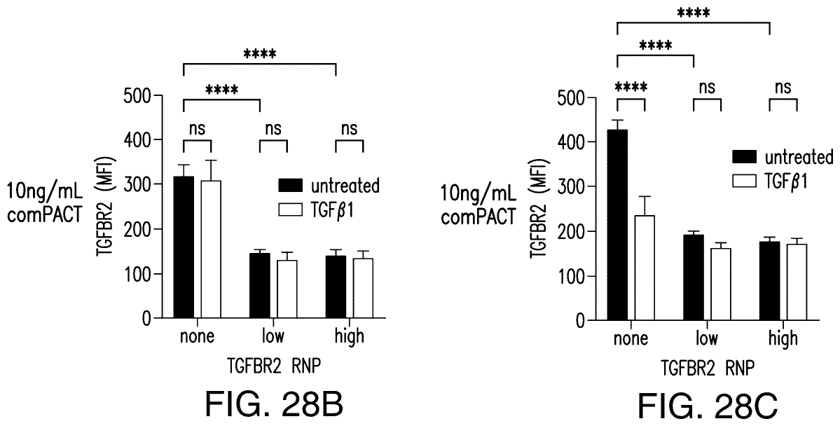

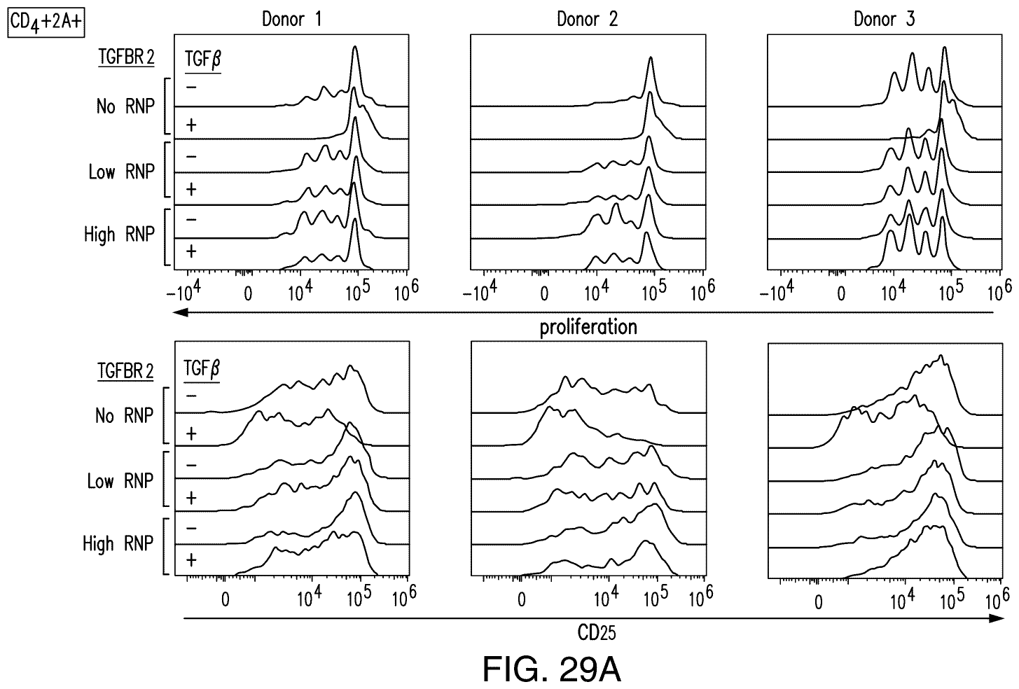

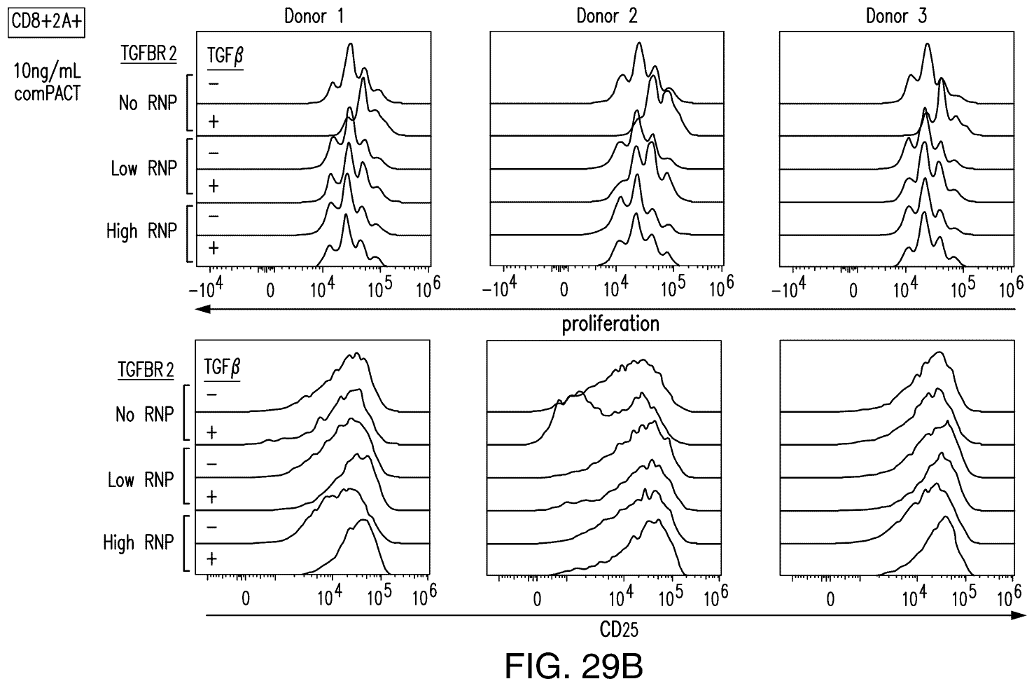

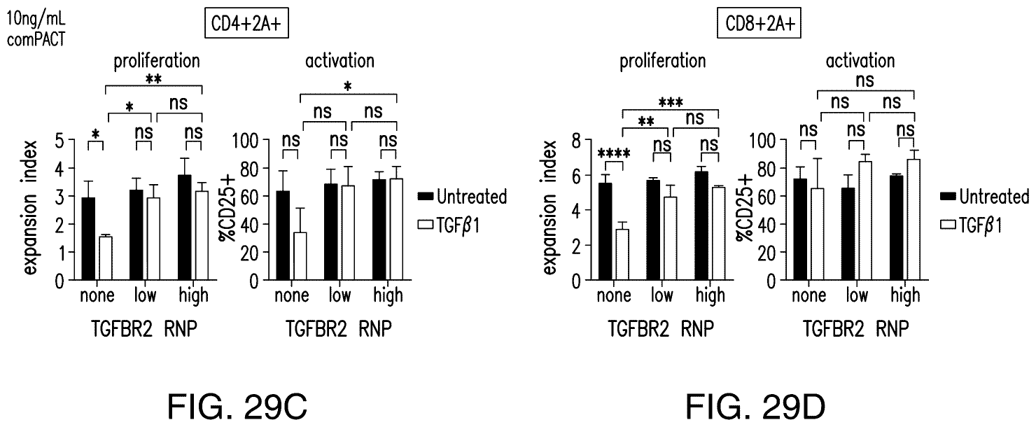

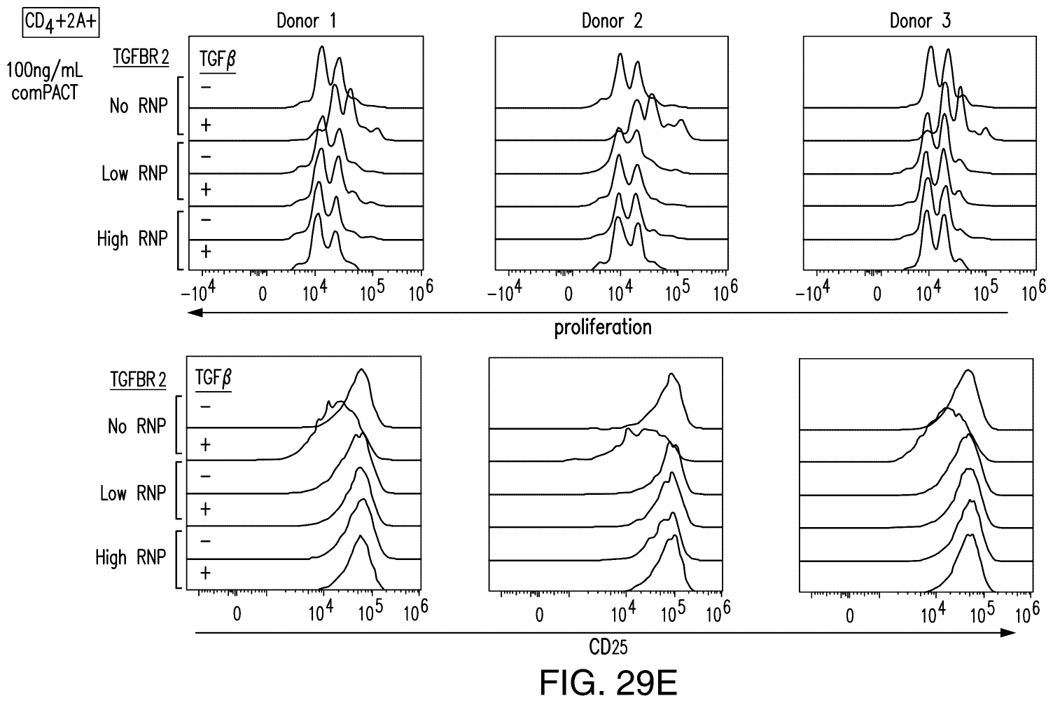

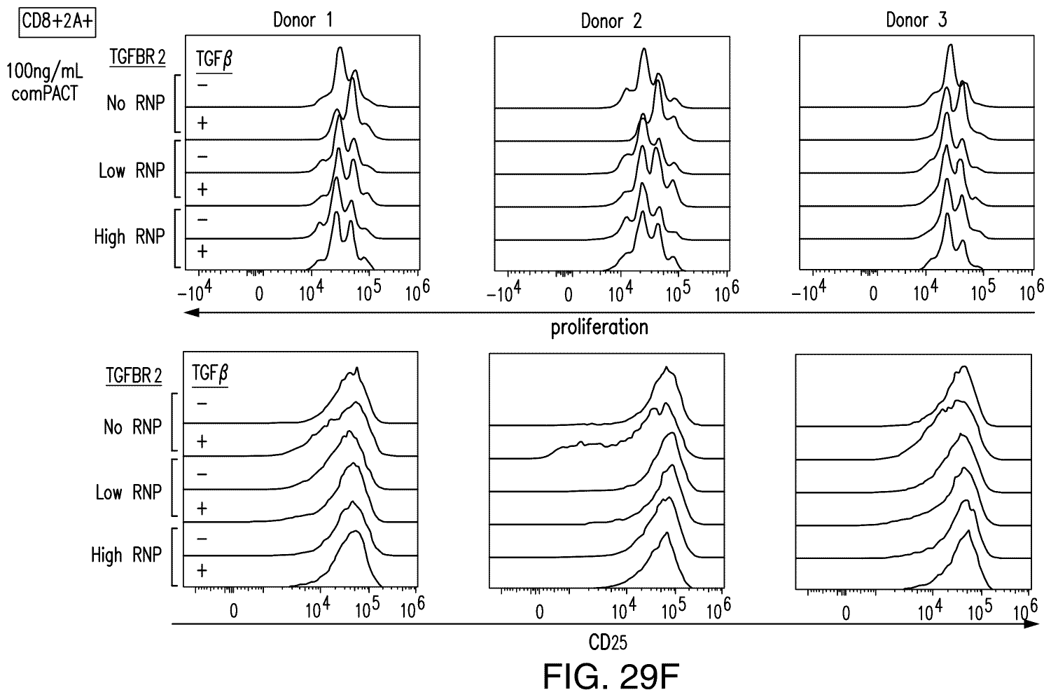

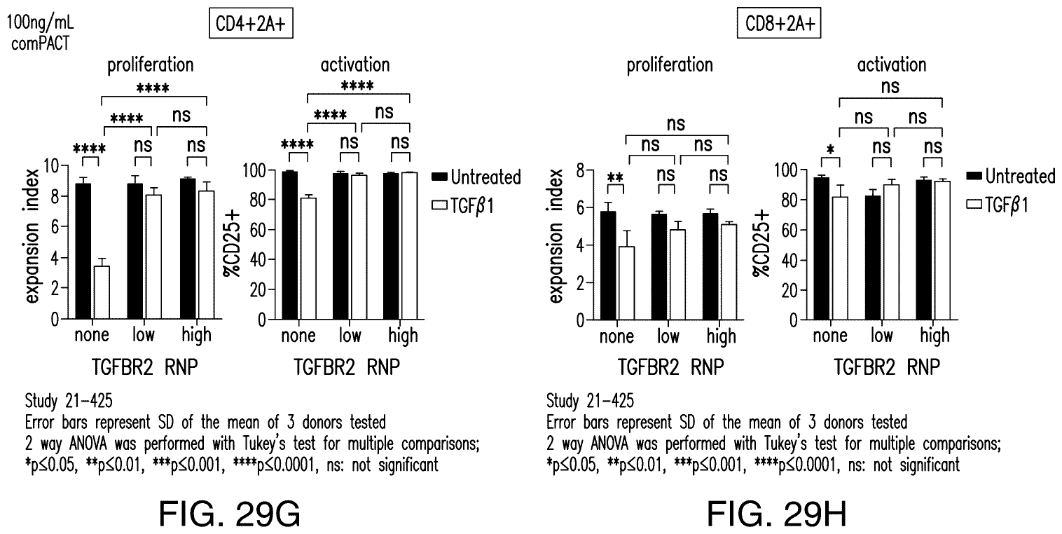

In the present example, the ability of different amounts of ribonucleoproteins (RNP) was tested. Cells were gated for their positivity to CD4 or CD8 receptors and proliferation, activation of the cells was determined. In addition, levels of TGFBRII were assessed. As shown in FIGS. 28A-28C, CD4 and CD8 T cells showed a significant reduction of the TGFBRII receptor as compared to the control using both tested RNP amounts. Notably, both proliferation and activation of T cells were rescued by the knockout of TGFBRII upon incubation with TGFβ. See FIGS. 29A-29H. These effects were comparable between the tested RNP amounts. In conclusion, the tested RNP conditions for TGFBR2 KO were sufficient to restore T cell proliferation and activation in the presence of TGFβ.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An immune cell, comprising:
a) an exogenous T cell receptor (TCR);
b) an exogenous CD8 receptor; and
c) a gene disruption of a TGFβRII locus.

2. The immune cell of claim 1, wherein the exogenous CD8 receptor comprises a first monomer and a second monomer.

3. The immune cell of claim 2, wherein each of the first monomer and the second monomer comprise a signal peptide, an extracellular domain, a transmembrane domain, and an intracellular domain.

4. The immune cell of claim 3, wherein
a) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8α signal peptide, an extracellular domain comprising a CD8α, a transmembrane domain comprising a CD8α, and an intracellular domain comprising a CD8α;
b) the first monomer comprises a signal peptide comprising a CD8α signal peptide, an extracellular domain comprising a CD8α, a transmembrane domain comprising a CD8α, and an intracellular domain comprising a CD8α; and the second monomer comprises a signal peptide comprising a CD8β signal peptide, an extracellular domain comprising a CD8β, a transmembrane domain comprising a CD8β, and an intracellular domain comprising a CD8β;
c) the first monomer comprises a signal peptide comprising a CD8α signal peptide, an extracellular domain comprising a CD8α, a transmembrane domain comprising a CD8α, and an intracellular domain comprising a CD8β; and the second monomer comprises a signal peptide comprising a CD8α signal peptide, an extracellular domain comprising a CD8α, a transmembrane domain comprising a CD8α, and an intracellular domain comprising a CD8β; or
d) the first monomer comprises a signal peptide comprising a CD8α signal peptide, an extracellular domain comprising a CD8α, a transmembrane domain comprising a CD8α, and an intracellular domain comprising a CD4; and the second monomer comprises a signal peptide comprising a CD8α signal peptide, an extracellular domain comprising a CD8α, a transmembrane domain comprising a CD8α, and an intracellular domain comprising a CD4.

5. The immune cell of claim 4, wherein
a) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12;
b) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 12; and the second monomer comprises a signal peptide comprising an amino acid sequence that is at least about 80% identical to the amino acid sequence set forth in SEQ ID NO: 13, an extracellular domain comprising an amino acid sequence that is at least about 80% identical to the amino acid sequence set forth in SEQ ID NO: 14, a transmembrane domain comprising an amino acid sequence that is at least about 80% identical to the amino acid sequence set forth in SEQ ID NO: 15, and an intracellular domain comprising an amino acid sequence that is at least about 80% identical to the amino acid sequence set forth in SEQ ID NO: 16;
c) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 16; or
d) the first monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17; and the second monomer comprises a signal peptide comprising an amino acid sequence that is at least about 80% identical to the amino acid sequence set forth in SEQ ID NO: 9, an extracellular domain comprising an amino acid sequence that is at least about 80% identical to the amino acid sequence set forth in SEQ ID NO: 10, a transmembrane domain comprising an amino acid sequence that is at least about 80% identical to the amino acid sequence set forth in SEQ ID NO: 11, and an intracellular domain comprising an amino acid sequence that is at least about 80% identical to the amino acid sequence set forth in SEQ ID NO: 17.

6. The immune cell of claim 1, wherein the gene disruption of the TGFβRII locus generates a knockout or a knockdown of the TGFβRII gene expression.

7. The immune cell of claim 1 further comprising a gene disruption of a TRAC locus and a TRBC locus.

8. The immune cell of claim 1, wherein the exogenous TCR is a patient derived TCR and/or wherein the exogenous TCR recognizes a patient derived cancer antigen.

9. The immune cell of claim 8, wherein the cancer antigen is a neoantigen or a private neoantigen.

10. The immune cell of claim 1, wherein the immune cell is a T cell, a Natural Killer T cell, a young T cell, a Natural Killer cell, or a lymphocyte.

11. A method of modifying a cell, the method comprising:
a) introducing into the cell a homologous recombination (HR) template nucleic acid sequence, wherein the HR template comprises:
i) first and second homology arms homologous to first and second target nucleic acid sequences of a TRAC locus or a TRBC locus;
ii) a TCR gene sequence positioned between the first and second homology arms; and
iii) a CD8 gene sequence positioned between the first and the second homology arms;
b) recombining the HR template nucleic acid into a TRAC or TRBC locus; and
c) generating a gene disruption of a TGFβRII locus.

12. The method of claim 11, wherein the gene disruption of the TGFβRII locus is generated by using a CRISPR/Cas system comprising a gRNA and a Cas9 nuclease, wherein the gRNA comprises a nucleic acid sequence set forth in SEQ ID NOs: 1-3.

13. A cell modified by the method of claim 11.

14. A composition comprising a cell of claim 1.

15. The composition of claim 14, wherein the composition is a pharmaceutical composition comprising a pharmaceutically acceptable excipient, a crystalloid solution, a cryopreservation agent, serum albumin, or a combination thereof.

16. A method of treating cancer in a subject in need thereof, the method comprising administering a therapeutically effective amount of the cell of claim 1.

17. The method of claim 16, wherein the cancer is a solid tumor or a liquid tumor.

18. The method of claim 17, wherein
a) the solid tumor is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer; and
b) the liquid tumor is selected from the group consisting of follicular lymphoma, leukemia, and multiple myeloma.

19. A kit comprising the cell of claim 1.
